(19) United States
(12) Patent Application Publication
(10) Pub. No.: US 2026/0043004 A1
(43) Pub. Date: Feb. 12, 2026

(54) METHODS OF MANUFACTURING CHIMERIC ANTIGEN RECEPTOR T CELLS

(71) Applicant: IMMPACT-BIO, LTD., Camarillo, CA (US)

(72) Inventors: Jenny Jiajia Cui, Camarillo, CA (US); Orit Foord, Camarillo, CA (US); James A. Johnston, Camarillo, CA (US); Deepthi Kolli, Camarillo, CA (US); Sylvain Philippe Roy, Camarillo, CA (US)

(21) Appl. No.: 18/998,278

(22) PCT Filed: Aug. 4, 2023
(86) PCT No.: PCT/US2023/071707

§ 371 Date: Jan. 24, 2025

Related U.S. Application Data
(60) Provisional application No. 63/370,487, filed on Aug. 4, 2022.
(60) Provisional application No. 63/370,489, filed on Aug. 4, 2022.

(51) Int. Cl. C12N 5/0783; C07K 14/725; C07K 16/28; C12N 15/86

(52) U.S. Cl. CPC C12N 5/0638; C07K 14/7051; C07K 16/2803; C07K 16/2887; C12N 15/86; C07K 2317/569; C12N 2501/2302; C12N 2501/2315; C12N 2740/15043

(57) ABSTRACT

The present disclosure relates to methods of manufacturing Chimeric Antigen Receptor (CAR) T cells. Notably, the manufacturing method omits the CD14+/CD25+ depletion step. Additionally, by starting with CD62L enrichment of naïve and memory T cells, the resulting CAT T cells show reduced release of proinflammatory cytokines. Furthermore, the transduced cells show improved quiescence after removal of the transactivation agent.

### CROSS-REFERENCE TO RELATED APPLICATIONS

This application claims benefit to U.S. Provisional Application No. 63/370,487, filed Aug. 4, 2022, and U.S. Provisional Application No. 63/370,489, filed Aug. 4, 2022, both of which are incorporated by reference herein in their entireties.

### REFERENCE TO AN ELECTRONIC SEQUENCE LISTING

The instant application contains a Sequence Listing, which has been submitted electronically in .xml format. The contents of the electronic sequence listing (_009718_00025_WO_SL.xml; Size: 74,512 bytes; and Date of Creation: Aug. 1, 2023) is herein incorporated by reference in its entirety.

### INCORPORATION BY REFERENCE

All publications, patents, and patent applications cited herein are incorporated by reference to the same extent as if each individual publication, patent, or patent application was specifically and individually indicates to be incorporated by reference. In the event of a conflict between a term herein and a term in an incorporated reference, the term herein controls.

### FIELD

The present disclosure relates to methods of manufacturing chimeric antigen receptor (CAR) T cells and the T cell populations prepared by the methods.

### BACKGROUND

Chimeric antigen receptors (CARs) are engineered molecules capable of redirecting the specificity of T cells to predetermined antigens, e.g., those predominantly expressed in cancer cells. The coding sequence for a CAR can be introduced into a T cell via a viral vector (e.g., a lentiviral or retroviral vector) to produce a chimeric antigen receptor (CAR) T cell. CAR T cells have been useful in the treatment of a variety of diseases, including but not limited to, cancers.

It is of vital importance to manufacture the intended CAR T cells in a safe and efficient fashion. For example, cytokine release syndrome (CRS), the most common type of toxicity caused by CAR T cells. CRS is a systemic inflammatory response caused by cytokines released by infused CAR T cells, ultimately resulting in widespread reversible organ dysfunction. Manufactured CAR T cells with lower toxicities are obviously more desirable.

Prior manufacturing processes relied upon open manufacturing steps (open system), including centrifugation, which made them vulnerable to contamination, reduced cellular function, and reduced manufacturing yield.

Accordingly, there is a need in the field to develop safer and more efficient processes to manufacture CAR T cells.

### SUMMARY OF THE INVENTION

Disclosed herein are methods of manufacturing Chimeric Antigen Receptor (CAR) T cells, and the CAR T cells manufactured therefrom. The manufacturing method may omit the CD14+/CD25+ depletion step. The manufacturing method may also omit using any transducing enhancer to introduce a viral construct into the T cells. By starting with CD62L enrichment of naïve and memory T cells, the resulting CAT T cells may show reduced release of proinflammatory cytokines. Furthermore, the transduced cells may show improved quiescence after the removal of the transactivation agent.

In one aspect, provided is a closed system method of manufacturing a composition comprising a population of T cells, said method comprising the steps of:
a) isolating CD62L+ cells from a starting population of cells, thereby obtaining a population of naïve/memory T (T_{N/MEM}) cells, wherein a depletion of CD14+/CD25+ cells is not performed;
b) contacting said population of T_{N/MEM} cells with a transactivating agent to obtain a population of activated T_{N/MEM} cells;
c) transducing said population of activated T_{N/MEM} cells with a viral construct to obtain a population of transduced cells, wherein transducing is performed in the absence of at least one transduction enhancer selected from the group consisting of: polybrene, protamine sulfate, LentiBoost™, Vectofusin-1, and poloxamer;
d) expanding said population of transduced cells; and
e) removing the transactivating agent.

In another aspect, the method may further comprise the following steps after step e):
f) expanding said population of transduced cells in the absence of said transactivating agent to obtain the composition comprising the population of T cells;
g) maintaining the composition comprising the population of T cells; and optionally
h) cryopreserving the composition comprising the population of T cells.
   Step g) of the method may be performed by placing the composition comprising the population of T cells in CTS™ OpTmizer™ media supplemented with IL-2 and IL-15 for about 48-144 hours, for example, about 48 hours, about 60 hours, about 72 hours, about 84 hours, about 96 hours, about 108 hours, about 120 hours, about 132 hours, or about 144 hours. Step h) of the method may be performed in CryoStor® CS10 media to achieve cryopreserving.

In another aspect, said starting population of cells of step a) may be obtained from peripheral blood mononuclear cells (PBMCs) collected from an individual by leukapheresis. Said population of T_{N/MEM} cells may be contacted with the transactivating agent for 18 to 48 hours, for example, about 18 hours, about 24 hours, about 30 hours, about 36 hours, about 42 hours, or about 48 hours, during step b) of the method. During step b) of the method, MACS® GMP T cell TransAct™ may be used as the transactivating agent. Additionally, said population of T_{N/MEM} cells is contacted with the transactivating agent in the presence of IL-2, IL-15, or both IL-2 and IL-15 during step b) of the method.

In yet another aspect, the viral construct of step c) can be a lentiviral construct comprising a nucleic acid encoding a chimeric antigen receptor (CAR). The CAR can be an anti-CD19/CD20 CAR comprising, in order:
i. an anti-CD20 scFv comprising (i) a light chain variable region having the amino acid sequence of SEQ ID NO: 34, and (ii) a heavy chain variable region having the amino acid sequences of SEQ ID NO: 35;
ii. a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
iii. an anti-CD19 scFv comprising (i) a heavy chain variable region having the amino acid sequence of SEQ ID NO: 36, and (ii) a light chain variable region having the amino acid sequences of SEQ ID NO: 37,
iv. a spacer domain comprising the amino acid sequence of any one selected from SEQ ID NOs: 21-29;
v. a transmembrane domain comprising the amino acid sequence of SEQ ID NO: 30 or 31;
vi. a 4-1BB cytoplasmic signaling domain comprising the amino acid sequence of SEQ ID NO: 32; and
vii. a CD3 zeta signaling domain comprising the amino acid sequence of SEQ ID NO: 33.
   The CAR may comprise the amino acid sequence of any one selected from SEQ ID NOs: 2, 4, 6, 8, 10, 12, 14, 16, 18, or 20. The CAR may comprise the amino acid sequence of SEQ ID NO: 2. During step c) of the method, transducing can be performed at a multiplicity of infection (MOI) in the range of 5-20, for example, at a MOI of about 5, about 10, about 15, or about 20. Transducing during step c) may be performed in the absence of protamine sulfate. Alternatively, transducing during step c) may be performed in the absence of a transduction enhancer.

In another aspect, expanding of step d) may be performed in the presence of IL-2 and IL-15 for about 24-120 hours, for example, about 24 hours, about 36 hours, about 48 hours, about 60 hours, about 72 hours, about 84 hours, about 96 hours, about 108 hours, or about 120 hours. During step d) of the methods, additional feed of IL-2 and IL-15 may be supplemented.

In yet another aspect, removing the transactivating agent during step e) is performed by washing with Sepax C-Pro. After step e) of the method, said population of transduced cells may show improved quiescence.

In a further aspect, provided is a population of T cells manufactured by the method.

Also provided is an anti-CD19/CD20 chimeric antigen receptor (CAR) comprising, in order:
i. an anti-CD20 scFv comprising (i) a light chain variable region having the amino acid sequence of SEQ ID NO: 34, and (ii) a heavy chain variable region having the amino acid sequences of SEQ ID NO: 35;
ii. a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
iii. an anti-CD19 scFv comprising (i) a heavy chain variable region having the amino acid sequence of SEQ ID NO: 36, and (ii) a light chain variable region having the amino acid sequences of SEQ ID NO: 37,
iv. a spacer domain comprising the amino acid sequence of any one selected from SEQ ID NOs: 21-29;
v. a transmembrane domain comprising the amino acid sequence of SEQ ID NO: 30 or 31;
vi. a 4-1BB cytoplasmic signaling domain comprising the amino acid sequence of SEQ ID NO: 32; and
vii. a CD3 zeta signaling domain comprising the amino acid sequence of SEQ ID NO: 33.

In one aspect, the CAR may comprise the amino acid sequence of any one selected from SEQ ID NOs: 2, 4, 6, 8, 10, 12, 14, 16, 18, or 20. The CAR may comprise the amino acid sequence of SEQ ID NO: 2.

Further provided is a nucleic acid encoding the CAR, as well as a T cell comprising the nucleic acid.

### BRIEF DESCRIPTION OF THE DRAWINGS

FIG. 1A depicts the anti-CD19/CD20 CAR expression achieved by transducing T_{N/MEM} cells on day 2 after TransAct™ activation with anti-CD19/CD20 CAR lentiviral vector at MOIs from 0.5 to 20 (see Example 2). Surface CAR expression was quantified on day 12 using an anti-mouse F(ab)2 antibody. % CAR expression (shown as the y-axis) was found to correlate with titration of MOI. At MOI of 5, about 60% CAR-positive cells were obtained. Data shown is an average of 3 donors.

FIG. 1B depicts the anti-CD19/CD20 CAR expression quantified at days 7, 10, and 12 post-TransAct™ activation (see Example 2). For all MOIs, CAR expression remained stable over 12 days. Data shown is an average of 3 donors.

FIG. 2 depicts the relationship between vector copy number (VCN; obtained by ddPCR) and % CAR expression (using flow cytometry) (see Example 3). As shown, VCN and % CAR expression correlated linearly as MOI increased from 0.5 to 20.

FIGS. 3A, 3B, and 3C collectively depict the relative cytotoxicity of anti-CD19/CD20 CAR T_{N/MEM} cells which were co-cultured with Raji cells (FIG. 3A: with both CD 19 and CD20 proteins; FIG. 3B: with CD 20 protein only; and FIG. 3C: with CD19 protein only) at a range of effector to target ratios for 48 hours. Cytotoxicity was measured by quantification of viable GFP+ target cells using flow cytometry (see Example 4).

FIGS. 4A, 4B, 4C, 4D, 4E, and 4F collectively depict the in vitro quantification of cytokine secretion from anti-CD19/CD20-CAR T cell titrates by ELISA. IL-2 (FIGS. 4A, 4B, and 4C) and IFN-γ (FIGS. 4D, 4E, and 4F) secretion by CAR-T cells which have been cocultured with target cells (Raji) overnight was assayed by ELISA (see Example 5). IL-2 and IFN-γ secretion from the anti-CD19/CD20-CAR T cell titrates with the E:T ratio and is significantly higher than anti-CD19-CAR T cells.

FIGS. 5A and 5B depict and cell expansion (FIG. 5A) and % CAR expression (FIG. 5B) for cell populations prepared using (a) no enhancer, (b) Lentiboost™, or (c) protamine sulfate.

FIG. 6 depicts a schematic representation of pALD based self-inactivating vectors. Abbreviations include: cPPT: central polypurine tract; HIV: Human Immunodeficiency Virus; LTR: long terminal repeat; RRE: REV response element; EGFRt: Epidermal Growth Factor Receptor truncated; WPRE: woodchuck post-transcriptional regulatory element. Symbols include: Δ: a 400-bp deletion in the 3′-LTR that completely removes the HIV enhancer and promoter sequences; Ψ: the packaging signal.

FIG. 7 depicts a map of a pALD_CD19/CD20 CAR plasmid. Arrows depict the direction of transcription.

FIG. 8 depicts a representative manufacturing scheme (12-day process) of the present disclosure.

FIG. 9 depicts CAR detection using anti-F(ab)2 antibodies and anti-EGFR antibodies in T cells transduced with CC310B vector (PC-3010-1221-2620) at MOIs ranging from 0.5 to 20. The T cells were obtained from three different donors (D1175, D1917, D6183). As shown in the figure, cells stained with anti-mouse F(ab)2 or anti-human EGFR behaved similarly at a range of MOIs, suggesting that anti-F(ab)2 antibody can be used to accurately and directly measure CAR expression level on the cell surface.

FIGS. 10A and 10B collectively depict a side-by-side comparison of enrichment processes performed with and without IgG, showing enrichment of different CD62L+ cell populations. For this, apheresis material from 2 donors (D6580: FIG. 10A; D8374: FIG. 10B) was processed side-by-side through the CD62L selection step with and without IgG. Cells were analyzed post CD62L+ enrichment step with and without IgG. Enriched cells were stained with antibodies and analyzed by flow cytometry. Staining was performed in triplicate. For donor D6580, Gammagard (Takeda) was used, while for donor D8374, Human IgG Affinity Purified Low Endotoxin (Innovative Research) was used.

FIG. 11 depicts the results of immunophenotyping at day 12 of cells enriched in the presence or absence of IgG. Abbreviations include: CAR: chimeric antigen receptor; SCM: T memory stem; IgG: Immune globulin; CM: central memory; EM: effector memory; Eff: effector; UTD: untransduced. Phenotyping of cells using antibodies against CD62L+, CD45RA, and CD45RO. Populations are defined as follows: SCM (CD45RA+, CD62L+), CM (CD45RA−, CD62L+), EM (CD45RA−, CD62L−), and effector (CD45RA+, CD62L−).

FIG. 12 depicts the level expansion of T cells with a representative process of the present disclosure. Cells from leukapheresis bag of D0597 were CD62L+ enriched only (triangles), or CD62L+ enriched and CD14+ depleted (circles). Cell count was performed at day 3, 7, 10, and 12 (day of transduction).

FIG. 13 depicts the CAR transduction efficiencies with and without protamine sulfate (PS). As shown, CAR transduction is higher in groups lacking protamine sulfate than in groups containing protamine sulfate.

FIGS. 14A and 14B depict the level of expansion (FIG. 14A) and total number of T cells (FIG. 14B) at day 12 undergoing a representative process of the present disclosure (PS: protamine sulfate).

FIGS. 15A, 15B, 15C, 15D, 15E, 15F, 15G, and 15H collectively depict the ELISA results of in vitro quantification of and IL-2 secretion (FIGS. 15A-D) and IFN-γ secretion (FIGS. 15E-H) of CAR T cells prepared by a representative process of the present disclosure from donor D0597. As shown, CAR T cell secretes both proinflammatory cytokines after co-culture with CD19 and/or CD20 positive Raji cells was characterized. Abbreviations include: CAR: chimeric antigen receptor; ELISA: enzyme-linked immunosorbent assay; IFN-γ: interferon gamma; IL-2: interleukin-2; UTD: untransduced T cells.

FIGS. 16A, 16B, 16C, 16D, 16E, and 16F depict the secretion of various cytokines by various cells. The cytokines include IFN-γ (FIG. 16A); IL-2 (FIG. 16B); IL-4 (FIG. 16C); TNF-α (FIG. 16D); IL-6 (FIG. 16E); and IL-10 (FIG. 16F). The results show that CD62L+ and T_{N/MEM} cells (“Tnm”) of prepared by an exemplary process of the present disclosure secrete less IFN-γ and IL-2 as compared to PBMCs.

FIGS. 17A, 17B, 17C, 17D, 17E, and 17F depict the inflammatory effect achieved. IFN-γ levels (FIGS. 17A-C) and IL-2 levels (FIGS. 17D-F) from T_{N/MEM} cells (Tnm) were compared with those from the PBMC. As shown, “CD19” means that cells were transduced with the anti-CD19 CAR encoding viral construct; while “CD1920” means that cells were transduced with the anti-CD19/CD20 CAR encoding viral construct.

FIGS. 18A, 18B, 18C, 18D, 18E, 18F, 18G, 18H, 18I, 18J, 18K, and 18L depict the IFN-γ secretion levels in anti-CD19/CD20 CAR T cells prepared from three different donors (D0597: FIGS. 18A-D; D6580: FIGS. 18E-H; and D1114: FIGS. 18I-L). As shown, increased secretion of IFN-γ by anti-CD19/CD20 CAR T cells was observed after o-culture with CD19 and/or CD20 positive Raji cells at increasing E:T ratios. These data demonstrate specific activity of both the anti-CD19 and anti-CD20 elements of anti-CD19/CD20-CAR of the present disclosure.

FIG. 19 depicts the surface expression of anti-CD19/CD20 CAR. 4 donors were transduced with anti-CD19/CD20 CAR constructs (Table 11), and CAR expression was quantified on day 12. Each point represents one donor, and the mean and SD are shown in the graph.

FIG. 20 depicts phenotype characterization of anti-CD19/CD20 CAR T cells containing various anti-CD19/CD20 CAR constructs. Phenotype data from one representative donor (D4096) were shown. Cells were stained using antibodies against CD62L and CD45RA and naïve, central memory, effector memory, and exhausted T cell populations are graphed for each construct.

FIGS. 21A, 21B, 21C, and 21D collectively depict the cytotoxic T lymphocyte (CTL) assay results showing the potencies of various anti-CD19/CD20 CAR constructs. CTL data from one representative donor (D4091) were shown. Anti-CD19/CD20 CAR T cells were co-cultured with luciferase-expressing target cells in RPMI media for 72 hours, and cytotoxicity was calculated based on target cell luminescence.

FIG. 22 depicts the IFN-γ secretion assay results showing the potencies of various anti-CD19/CD20 CAR constructs. IFN-γ data from one representative donor (D4096) were collected at 5:1 (E:T). CAR T cells were incubated with target cells for 24 hours, and supernatant was analyzed using CBA assay.

FIGS. 23A and 23B depict phenotype characterization of anti-CD19 CAR T cells and IMPT-514. Abbreviations include: Eff/Exh: effector/exhausted; and LN: lupus nephritis. PBMCs isolated from 2 healthy donors (D136 and D292) and 2 LN donors (PM04 and SB07) were transduced with CC352 and harvested on Day 9 (CD19 CAR T). CD62L-enriched cells were transduced with CC314B and harvested on Day 12 (IMPT-514). Samples were stained and measured in triplicates and mean and standard deviations are graphed.

FIGS. 24A, 24B, and 24C depict cytotoxic T lymphocyte assay results. Anti-CD19 CAR T cells and IMPT-514 were co-cultured with Raji B cells at various effector to target (E:T) ratios, and cytotoxicity was measured. Cytotoxicity, measured by Raji cell viability 72 hours after co-culture, did not demonstrate significant differences between IMPT-514 and anti-CD19 CAR T cells across all three donors.

FIGS. 25A, 25B, 25C, and 25D depict the characterization of IFN-γ secretion in Raji B cell co-culture assay. Anti-CD19 CAR T cells and IMPT-514 were co-cultured with Raji B cells at various effector to target (E:T) ratios, and IFN-γ secretion was measured. IFN-γ secretion, measured by Raji cell viability 72 hours after co-culture, did not demonstrate significant differences between IMPT-514 and anti-CD19 CAR T cells across all donors. Abbreviations include: UTD: untransduced; and IFN-γ: interferon gamma. PBMCs isolated from healthy and LN donors were transduced with CC352 and harvested on Day 9 (anti-CD19 CAR T cells). CD62L enriched cells were transduced with CC314B and harvested on Day 12 (IMPT-514). CAR T cells were co-cultured with Raji B cells starting at an effector to target (E:T) ratio of 10 to 1 and titrated down. 24 hours after co-culture, the supernatant was removed and secreted IFN-γ were measured using Th1/Th2/Th17 cytometric bead array (CBA) kit.

FIGS. 26A and 26B depict CD25 expression levels of IMPT-514 and anti-CD19 CAR T cells. Cryopreserved IMPT-514 and anti-CD19 CAR T cells were thawed, and cells stained using an anti-CD25 antibody. Stained cells were analyzed using a flow cytometer (Attune). Mean fluorescence intensity (MFI) was reported

FIG. 27 depicts surface CAR expression of transduced PBMCs and CD62L-enriched cells from two donors. PBMCs and CD62L+ cells were isolated from 3 donors (D1175, D1858, D6135) were transduced with anti-CD19 CAR and harvested on Day 12. CAR expression was quantified using anti-FMC63 antibody. As shown, CAR expression was comparable for anti-CD19 CAR manufactured from PBMC or CD62L+ cells.

FIGS. 28A and 28B depict cytotoxic T lymphocyte assay results of anti-CD19 CAR T cells produced from PBMCs and CD62L-enriched cells.

FIGS. 29A and 29B depict the characterization of IFN-γ secretion in Raji B cell co-culture assay with anti-CD19 CAR T cells produced from PBMCs and CD62L-enriched cells.

FIG. 30 depicts a schematic illustration of cytokine release syndrome (CRS), showing the interaction of target cells, T cells, and macrophages.

FIG. 31 depicts another representative manufacturing scheme (8-day process) of the present disclosure.

FIG. 32 depicts the cell subset distribution between apheresis and post-CD62L enrichment as discussed in Example 13.

FIG. 33 depicts the comparison of Cumulative Fold Expansion (CFE) for each of the 12-day process and the 8-day process.

FIG. 34A depicts the CD3 expression in cell populations resulting from the 12-day process and the 8-day process. FIG. 34B depicts the CAR expression in cell populations resulting from the 12-day process and the 8-day process.

FIGS. 35A and 35B depict the vector copy number (VCN) comparison. FIG. 35A shows the VCN based upon Leu 16, while FIG. 35B shows the VCN based upon FMC63.

FIGS. 36A, 36B, and 36C collectively depict the characterization of IFN-γ secretion. IFN-γ secretion was compared from cell populations resulting from the 12-day process and the 8-day process, targeting the wild-type (WT) (FIG. 36A), CD20KO (FIG. 36B), and CD19KO (FIG. 36C) cell lines.

FIG. 37 depicts the phenotypical characterization of T cell subsets in the cell populations resulting from the 12-day process and the 8-day process.

FIG. 38 depicts the T cell activation level by measuring T cell activation markers (CD25 and CD69) on days 1, 2, 3, 7, 10, and 12 of the manufacturing process.

FIG. 39 depicts the T cell activation level by measuring cell size as a marker of T cell activation.

### DETAILED DESCRIPTION

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., +/−10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The term “Administering” as used herein refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms, “activated” and “activation” refer to the state of a T cell that has been sufficiently stimulated to induce detectable cellular proliferation. In one embodiment, activation may also be associated with induced cytokine production, and detectable effector functions. The term “activated T cells” refers to, among other things, T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation may be evidenced by proliferation and/or cytokine production by T cells that have received a primary activation signal, such as stimulation through the TCR/CD3 complex.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as V_{H}) and a heavy chain constant region. The heavy chain constant region comprises three constant domains, CH1, CH2 and CH3. Each light chain comprises a light chain variable region (abbreviated herein as V_{L}) and a light chain constant region. The light chain constant region comprises one constant domain, CL. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

The term “human antibody” is intended to comprise antibodies having variable and constant domain sequences generated, assembled, or derived from human immunoglobulin sequences, or sequences indistinguishable therefrom. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fc), generally that of a human immunoglobulin constant domain. In some embodiments, a humanized antibody may comprise a CH1, hinge, CH2, CH3, and, optionally, a CH4 region of a human heavy chain constant domain.

Antibodies can include, e.g., monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies, Anticalins™, and the like.

An immunoglobulin may derive from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

Amino acid sequences that specifically bind to desired antigens are known in the art or may be prepared using methods known in the art. Examples include immunoglobulins, variable regions of immunoglobulins (e.g., variable fragment (“Fv”) or bivalent variable fragment (“Fab”)), single chain antibodies, etc. In certain embodiments, the antigen binding molecule is an antibody fragment that specifically binds to the antigen, including one or more of the complementarity determining regions (CDRs) thereof. In further embodiments, the antigen binding molecule is a single chain variable fragment (scFv).

In some instances, a CDR can be substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g., 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 95%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody. In various embodiments, an antigen binding fragment cross-competes with the reference antibody, for example, binding to substantially the same or identical epitope as the reference antibody.

The terms “variable region” or “variable domain” are used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “V_{L}” and “V_{L} domain” are used interchangeably to refer to the light chain variable region of an antibody or an antigen-binding molecule thereof.

The terms “V_{H}” and “V_{H} domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

The term “heavy chain” when used in reference to an antibody can refer to any distinct type, e.g., alpha, delta, epsilon, gamma, and mu, based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

The term “light chain” when used in reference to an antibody can refer to any distinct type, e.g., kappa or lambda based on the amino acid sequence of the constant domains. Light chain amino acid sequences are well known in the art. In specific embodiments, the light chain is a human light chain.

The term “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens.

The term “autologous” refers to any material derived from the same individual to which it is later to be re-introduced. For example, engineered autologous cell therapy herein involves collection of lymphocytes from a patient, which are then engineered to express, e.g., a CAR construct, and then administered back to the same patient.

The term “binding affinity” generally refers to the strength of the sum total of non-covalent interactions between a single binding site of a molecule (e.g., an antibody) and its binding partner (e.g., an antigen). Unless indicated otherwise “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of k_{off}/kₒₙ. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIACORE™ or KinExA™.

The term “K_{D}” (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e., stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (K_{D}) of a particular molecule (e.g., antibody) to its interactive partner molecule (e.g., antigen X) compared to the binding affinity of the molecule (e.g., antibody) to another interactive partner molecule (e.g., antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{A}” refers to the association rate constant of a particular antibody-antigen interaction, or the association rate constant of an antibody or antibody-binding fragment.

The term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of the present disclosure include, but are not limited to, cancers of the immune system including lymphoma, leukemia, myeloma, and other leukocyte malignancies. In some embodiments, the methods of the present disclosure can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer, squamous cell cancer, T cell lymphoma, environmentally induced cancers including those induced by asbestos, other B cell malignancies, and combinations of said cancers. In one particular embodiment, the cancer is multiple myeloma. The particular cancer can be responsive to chemo- or radiation therapy or the cancer can be refractory. A refractory cancer refers to a cancer that is not amendable to surgical intervention and the cancer is either initially unresponsive to chemo- or radiation therapy or the cancer becomes unresponsive over time. Cancer further includes relapsed or refractory large B-cell lymphoma after two or more lines of systemic therapy, including diffuse large B-cell lymphoma (DLBCL) not otherwise specified, primary mediastinal large B-cell lymphoma after two or more lines of systemic therapy, high grade B-cell lymphoma, and DLBCL arising from follicular lymphoma.

During autoimmunity, tissue damage may be mediated by the effector actions of T cells and/or B cells. The antigen (or group of antigens) against which the autoimmune response is directed, and the mechanism by which the antigen-bearing tissue is damaged, together determine the pathology and clinical expression of the autoimmune disease. In a “B cell mediated autoimmune disease,” tissue injury is caused by antibody (e.g., IgM and/or IgG) responses to autoantigens located on cell surfaces or extracellular matrix, immune complexes containing autoantibodies to soluble autoantigens, or binding of autoantibodies to a cell-surface receptor that either stimulates the receptor or blocks its stimulation by its natural ligand. Non-limiting examples of “B cell mediated autoimmune disease” include: Addison's disease; autoimmune hemolytic anemia; azoospermia; celiac disease; Goodpasture's syndrome; Grave's Disease; Hashimoto's thyroiditis; immune thrombocytopenic purpura; myasthenia gravis; neuromyelitis optica; Pemphigus vulgaris/foliaceus; primary biliary cirrhosis; rheumatic heart disease/post streptococcal glomerulonephritis; systemic lupus erythematosus; multiple sclerosis; rheumatoid arthritis; myositis; systemic sclerosis, ANCA-vasculitis; and Sjögren's syndrome.

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1.alpha. (MIP-1.alpha., MIP-1a), MIP-1.beta. (MIP-1b), gamma-induced protein 10 (IP-10), and thymus and activation regulated chemokine (TARC or CCL17).

The terms “Chimeric Antigen Receptor” or “CAR” refer to a molecule engineered to comprise a binding motif and a means of activating immune cells (for example T cells such as naïve T cells, central memory T cells, effector memory T cells or combination thereof), NK cells, and other cell types upon antigen binding. CARs are also known as artificial T cell receptors, chimeric T cell receptors or chimeric immunoreceptors. In some embodiments, a CAR comprises a binding motif, an extracellular domain, a transmembrane domain, one or more costimulatory domains, and an intracellular signaling domain. A T cell that has been genetically engineered to express a chimeric antigen receptor may be referred to as a CAR T cell.

“Extracellular domain” (or “ECD”) refers to a portion of a polypeptide that, when the polypeptide is present in a cell membrane, is understood to reside outside of the cell membrane, in the extracellular space.

The binding domain of the CAR may be followed by a “spacer,” which refers to the region that moves the antigen binding domain away from the effector cell surface to enable proper cell/cell contact, antigen binding and activation. The spacer may further comprise a hinge region or domain. The hinge region is typically membrane proximal, and is between the transmembrane (TM) and the binding domain. In certain embodiments, a hinge region is an immunoglobulin hinge region and may be a wild-type immunoglobulin hinge region or an altered wild-type immunoglobulin hinge region. Other exemplary hinge regions used in the CARs described herein include the hinge region derived from the extracellular regions of type 1 membrane proteins such as IgG (e.g., IgG1, IgG2, IgG3, and IgG4), CD8α, CD4, CD28, 4-1BB, and CD7, which may be wild-type hinge regions from these molecules or may be altered.

The “transmembrane” region or domain is the portion of the CAR that anchors the extracellular binding portion to the plasma membrane of the immune effector cell. The transmembrane domain may be for example those obtained from CD8α, CD4, CD28, CD45, CD9, CD16, CD22, CD33, CD64, CD80, CD86, CD134, CD137, CD3zeta, and CD154. In one embodiment, the transmembrane domain is the transmembrane domain of CD8α. In certain embodiments, the transmembrane domain is synthetic in which case it would comprise predominantly hydrophobic residues such as leucine and valine.

The “intracellular domain” comprises one or more costimulatory domain, and one or more intracellular signaling domains. The intracellular costimulatory domain can be from, e.g., 4-1BB and/or CD28.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or help or activity including the secretion of a cytokine. Thus, the terms “intracellular signaling domain” or “signaling domain,” are used interchangeably herein and refer to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains. The intracellular signaling domain can be, e.g., derived from CD3z.

It is known that signals generated through the T cell receptor alone are generally insufficient for full activation of the T cell and that a secondary, or costimulatory signal, is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the disclosure include those derived from CD3 zeta, FeR gamma, FcR beta, CD3 gamma, CD3 delta, CD3 epsilon, CD5, CD22, CD79a, CD79b, and CD66d.

As used herein, the term, “costimulatory signaling domain,” or “costimulatory domain”, refers to the portion of the CAR comprising the intracellular domain of a costimulatory molecule. “Costimulatory molecules” are cell surface molecules other than antigen receptors or Fc receptors that provide a second signal required for efficient activation and function of T lymphocytes upon binding to antigen. The inclusion of one or more costimulatory signaling domains may enhance the efficacy and expansion of T cells expressing CAR receptors. The intracellular signaling and costimulatory signaling domains may be linked in any order in tandem to the carboxyl terminus of the transmembrane domain. Suitable costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSFi4), LTBR, Ly9 (CD229), lymphocyte function-associated antigen-1 (LFA-1 (CD11a/CD18), MEW class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMFi; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

As will be appreciated, although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Thus, CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients. See Milone et al., Molecular Therapy, 2009; 17: 1453-1464; Zhong et al., Molecular Therapy, 2010; 18: 413-420 and Carpenito et al., PNAS, 2009; 106:3360-3365).

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution.

“Combination therapy” refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

The “control elements” or “regulatory sequences” present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters maybe used.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

An “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

“Endogenous” with reference to a gene, protein, and/or nucleic acid refers to the natural presence of that gene, protein, and/or nucleic acid in a cell, such as an immune cell.

“Exogenous” refers to an introduced agent, such as a nucleic acid, gene, or protein, into a cell, for example from an outside source. A nucleic acid introduced into a cell is exogenous even if it encodes a protein which is naturally found in the cell. Such exogenous introduction of a nucleic acid encoding a protein can be used to increase the expression of the protein over the level that would naturally be found in the cell under similar conditions, e.g., without introduction of the exogenous nucleic acid.

A “fragment” or “portion” of a material or entity as described herein has a structure that comprises a discrete portion of the whole, e.g., of a physical entity or abstract entity. In some embodiments, a fragment lacks one or more moieties found in the whole. In some embodiments, a fragment consists of or comprises a characteristic structural element, domain or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer (e.g., 85-90%, 85-95%, 85-100%, 90-95%, 90-100%, or 95-100%). The whole material or entity may in some embodiments be referred to as the “parent” of the fragment.

The term “fusion polypeptide” or “fusion protein” generally refers to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man. In embodiments, a CAR is a fusion protein.

A “T cell receptor” or “TCR” refers to antigen-recognition molecules present on the surface of T cells. During normal T cell development, each of the four TCR genes, alpha, beta, gamma, and delta, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

The term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies or NK cell therapies. T cell therapy can include adoptive T cell therapy, tumor-infiltrating lymphocyte (TIL) immunotherapy, autologous cell therapy, and allogeneic T cell transplantation.

One of skill in the art will recognize techniques to enhance the effectiveness of cell therapy using, e.g., preconditioning techniques such as those found in U.S. Pat. Nos. 9,855,298 and 10,322,146, the contents of which are hereby incorporated by reference in their entirety.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “in vitro” refers to events occurring in an artificial environment, e.g., in a test tube, reaction vessel, cell culture, etc., rather than within a multi-cellular organism. The term “in vitro cell” refers to any cell which is cultured ex vivo. In particular, an in vitro cell can include a T cell. The term “in vivo” refers to events that occur within a multi-cellular organism, such as a human or a non-human animal.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%). A substance is “pure” if it is substantially free of other components or of contaminants. In some embodiments, a substance may still be considered “isolated” or even “pure,” after having been combined with certain other components such as, for example, one or more carriers or excipients (e.g., buffer, solvent, water, etc.); in such embodiments, percent isolation or purity of the substance is calculated without comprising such carriers or excipients.

The term “lymphocyte” includes natural killer (NK) cells, T cells, or B cells. NK cells are a type of cytotoxic (cell toxic) lymphocyte that represent a component of the inherent immune system. NK cells reject tumors and cells infected by viruses. It works through the process of apoptosis or programmed cell death. They were termed “natural killers” because they do not require activation in order to kill cells. T cells play a role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory T_{SCM} cells, like naïve cells, are CD45RO⁻, CCR7+, CD45RA+, CD62L+ (L-selectin), CD27+, CD28+ and IL-7R alpha⁺, but they also express large amounts of CD95, IL-2RB, CXCR3, and LFA-1, and show numerous functional attributes distinctive of memory cells); (ii) central memory T.sub.CM cells express L-selectin and the CCR7, they secrete IL-2, but not IFN-gamma or IL-4, and (iii) effector memory TEM cells, however, do not express L-selectin or CCR7 but produce effector cytokines like IFN-gamma and IL-4), Regulatory T cells (Tregs, suppressor T cells, or CD4+CD25+ regulatory T cells), Natural Killer T cells (NKT) and Gamma Delta T cells. B-cells, on the other hand, play a role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

The term “operably linked” refers to a juxtaposition where the components described are in a relationship permitting them to function in their intended manner. For example, a control element “operably linked” to a functional element is associated in such a way that expression and/or activity of the functional element is achieved under conditions compatible with the control element.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In other embodiments, a vector for use in practicing the embodiments described herein including, but not limited to expression vectors and viral vectors, will include exogenous, endogenous, or heterologous sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” sequence is an exogenous sequence that may be from a different protein of the same species or a different species than the protein or cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances may function independent of their orientation relative to another control sequence. An enhancer may function cooperatively or additively with promoters and/or other enhancer elements. The term “promoter/enhancer” refers to a segment of DNA which contains sequences capable of providing both promoter and enhancer functions.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Regulatory T cells” (“Treg”, “Treg cells”, or “Tregs”) refer to a lineage of CD4+T lymphocytes that participate in controlling certain immune activities, e.g., autoimmunity, allergy, and response to infection. Regulatory T cells may regulate the activities of T cell populations, and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4+T lymphocytes. However, Treg cells within a tumor microenvironment (i.e., tumor-infiltrating Treg cells), Treg cells may make up as much as 20-30% of the total CD4+T lymphocyte population.

“Single chain variable fragment”, “single-chain antibody variable fragments” or “scFv” antibodies refer to forms of antibodies comprising the variable regions of only the heavy and light chains, connected by a linker peptide.

The phrase “therapeutic agent” may refer to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms or human subjects. In some embodiments, an appropriate population may be defined by various criteria, such as a certain age group, gender, genetic background, preexisting clinical conditions, in accordance with presence or absence of a biomarker, etc. In some embodiments, a therapeutic agent is a substance that may be used to alleviate, ameliorate, relieve, inhibit, prevent, delay onset of, reduce severity of, and/or reduce incidence of one or more symptoms or features of a disease, disorder, and/or condition. In some embodiments, a therapeutic agent is an agent that has been or is required to be approved by a government agency before it may be marketed for administration to humans. In some embodiments, a therapeutic agent is an agent for which a medical prescription is required for administration to humans.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, an RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” or “lentiviral vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to: HIV (human immunodeficiency virus; including HIV type-1, and HIV type-2); visna-maedi virus (VMV) virus; the caprine arthritis encephalitis virus (CAEV); equine infectious anemia virus (EIAV); feline immunodeficiency virus (FIV); bovine immune deficiency virus (BIV); and simian immunodeficiency virus (SIV).

In some embodiments, compositions contemplated herein comprise an effective amount of an expanded modified T cell composition, alone or in combination with one or more therapeutic agents. Thus, the T cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics and anti-viral agents. Such therapeutic agents may be accepted in the art as a treatment for a disease state as described herein, such as a cancer. In one embodiment the compositions contemplated herein may also be administered with inhibitors of TGF-.beta., for example the small molecule inhibitor LY55299. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

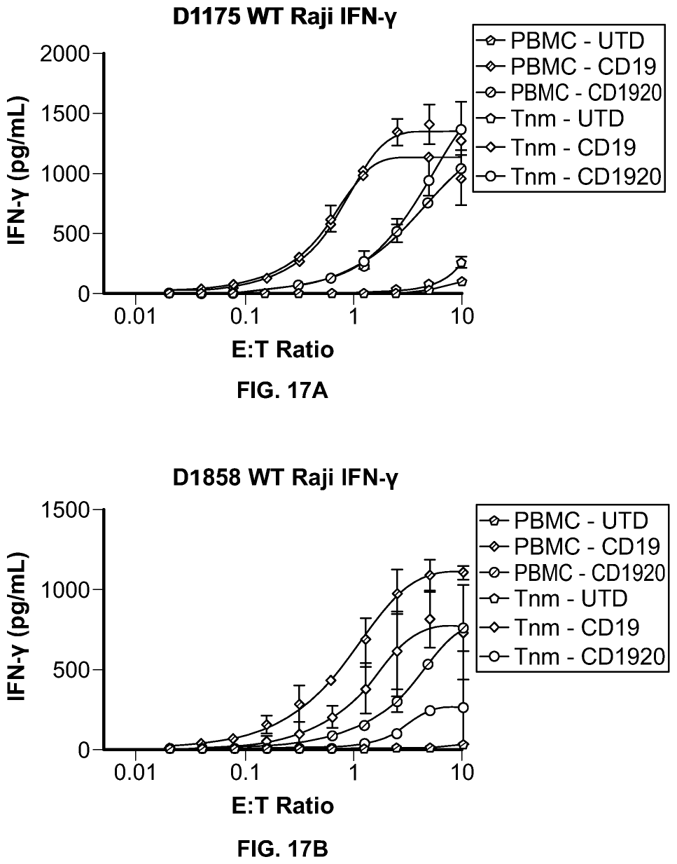

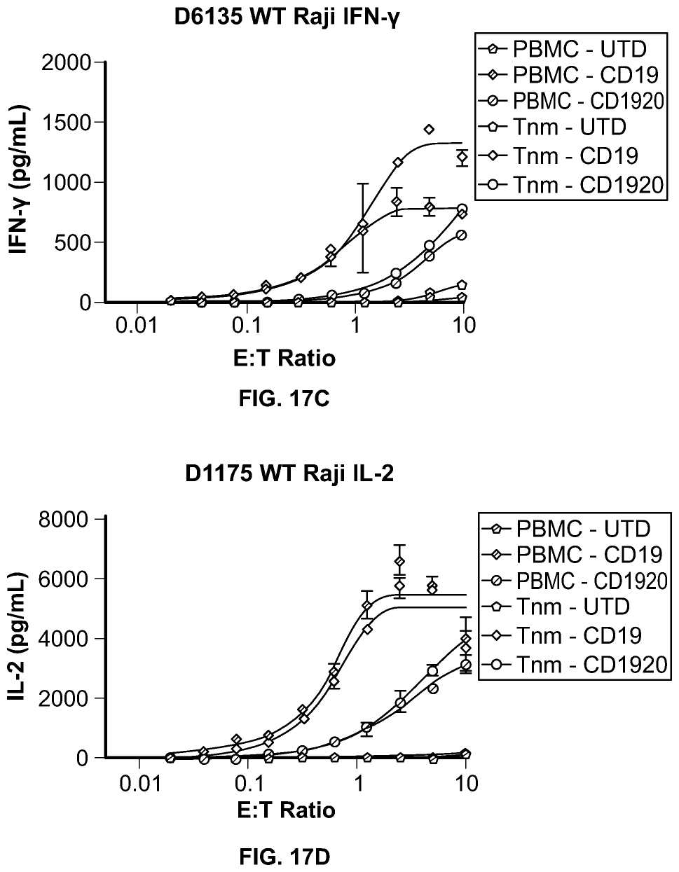

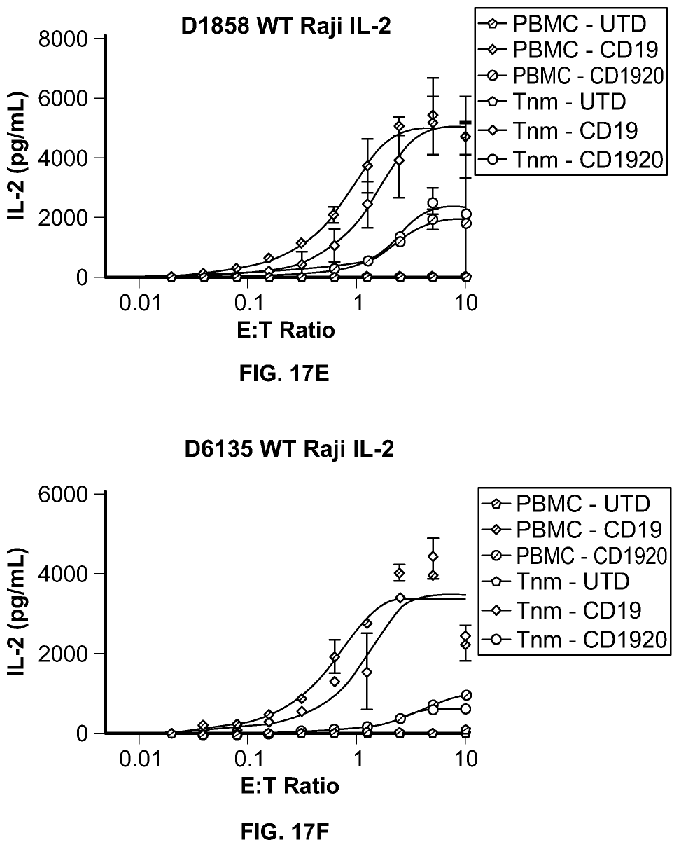

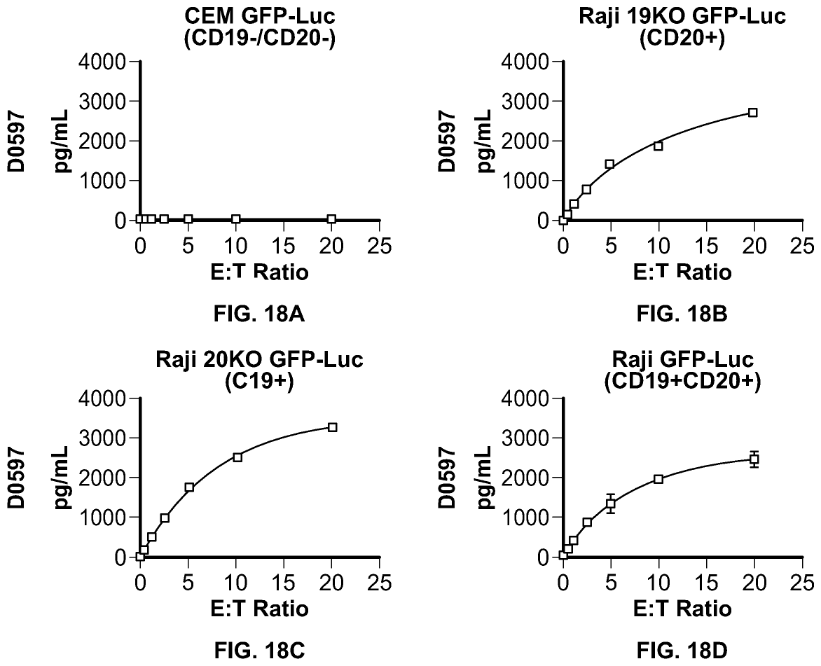

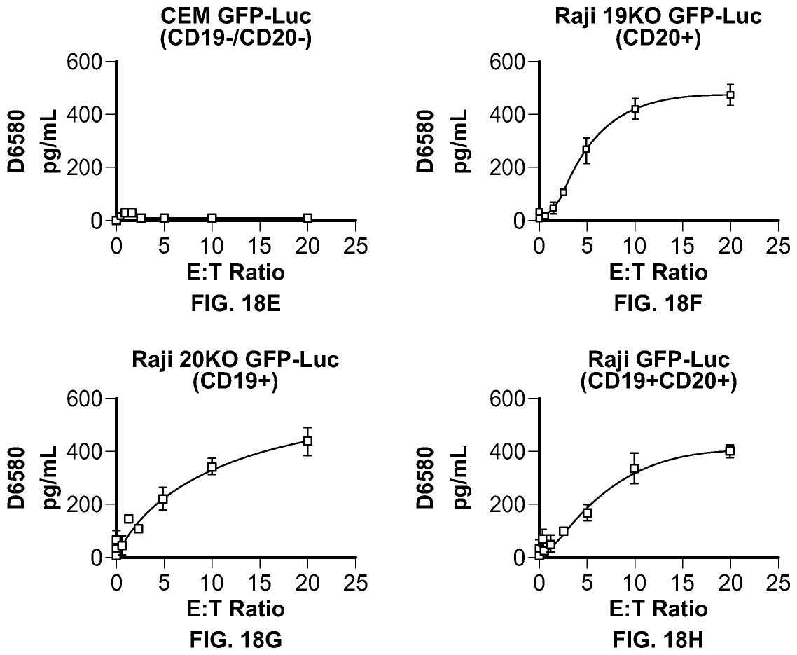

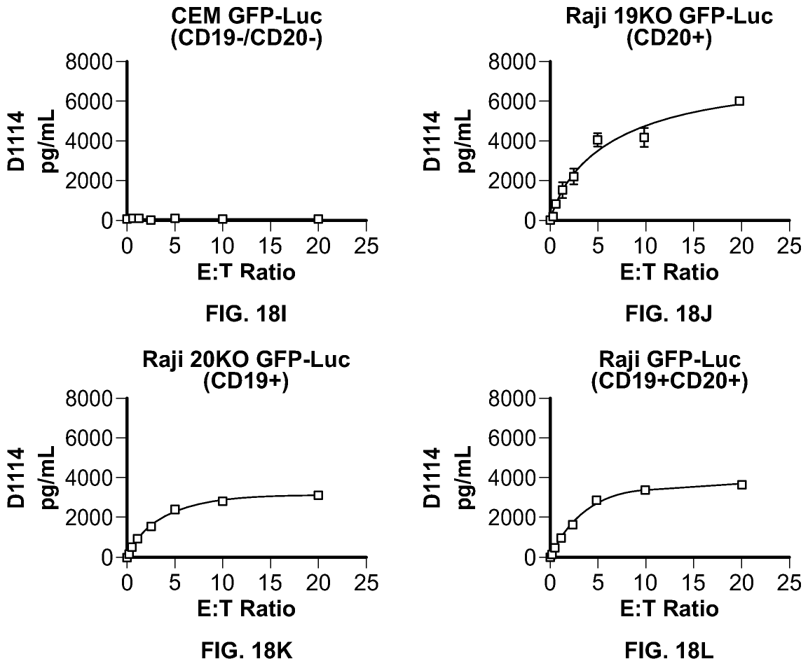

In an aspect of the present invention, it is preferred that an inflammatory effect or response is reduced relative to existing constructs and methods of treatment. See, e.g., FIGS. 17A-F and FIGS. 18A-L.

For example, the inflammatory effect or response is reduced as compared to tisagenlecleucel (Kymriah) or axicabtagene ciloleucel (Yescarta).

In certain embodiments, a construct described herein does not produce or otherwise elicit an inflammatory response.

In certain embodiments, a construct described herein produces or otherwise elicits an inflammatory response which is less than tisagenlecleucel (Kymriah) or axicabtagene ciloleucel (Yescarta).

In certain embodiments, a therapeutic method described herein does not produce or otherwise elicit an inflammatory response in a subject.

In certain embodiments, a therapeutic method described herein produces or otherwise elicits an inflammatory response in a subject which is less than tisagenlecleucel (Kymriah) or axicabtagene ciloleucel (Yescarta).

In an aspect, the inflammatory effect or response is reduced as indicated by reduced levels of interferon gamma, while maintaining substantially the same cytotoxicity.

In an aspect, the inflammatory effect or response is reduced as indicated by reduced levels of interferon gamma and IL-2, while maintaining substantially the same cytotoxicity.

In certain embodiments, the inflammatory effect or response is reduced as indicated by reduced levels of interferon gamma, as compared to the inflammatory effect or response produced by CAR-T cells made using a CD3+ T-cell population (e.g., tisagenlecleucel (Kymriah) or axicabtagene ciloleucel (Yescarta)), while maintaining substantially the same cytotoxicity.

In certain embodiments, compositions comprising T cells contemplated herein may be administered in conjunction with any number of chemotherapeutic agents. Illustrative examples of chemotherapeutic agents include but are not limited to alkylating agents such as thiotepa and cyclophosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (Taxol™, Bristol-Myers Squibb, Princeton, N.J.) and doxetaxel (Taxotere™, Sanofi, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RPS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of therapeutic agents may be used in conjunction with the compositions described herein. In one embodiment, the composition comprising T cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

It will be appreciated that certain combination therapies have become state of the art for various cancer types.

For examples, it will be appreciated that more common treatment options include a monoclonal antibody (such as rituximab or obinutuzumab) combined with one or more chemotherapy agents as described herein. The chemotherapy can be a single agent (such as bendamustine) or a combination of drugs, such as the CHOP (cyclophosphamide, doxorubicin, vincristine, prednisone) or CVP (cyclophosphamide, vincristine, prednisone) regimens as described herein.

One such combination therapy thus includes, for example, R-CHOP, an immunochemotherapy regimen consisting of rituximab, cyclophosphamide, hydroxydaunorubicin hydrochloride (doxorubicin hydrochloride), vincristine (Oncovin) and prednisone used to treat both indolent and aggressive forms of non-Hodgkin lymphoma. See, e.g., R-CHOP regimen at the national Cancer Institute website (https://www.cancer.gov/publications/dictionaries/cancer-drug/def/r-chop-regimen), the contents of which are hereby incorporated by reference in their entirety.

Additional combination treatments include for example dose-adjusted etoposide, doxorubicin and cyclophosphamide with vincristine, prednisone and rituximab (known as “DA-EPOCI-R”). Other regimens known in the art include CVP (cyclophosphamide, vincristine, prednisone) regimens. Other art-known regiments include Hyper-CVAD (cyclophospharnide, vincristine, doxorubicin (Adriamycin), and dexamethasone, alternating with high-dose methotrexate plus cytarabine); “Dose-intensified” R-CHOP (rituximab, cyclophosphamide, doxorubicin, vincristine, and prednisone), alternating with rituximab and cytarabine, and RDHAP (Rituximab, dexamethasone, cytarabine, cisplatin). Also suitable is CODOX-M (cyclophosphanide, vincristine [Oncovin], doxorubicin, and high-dose methotrexate), alternating with IVAC (ifosfamide, etoposide [VP-16], and cytarabine. Other suitable regimens will be known to those in the art for use in accordance with the invention.

One skilled in the art will also appreciate that the current chimeric receptors can be administered in combination with one or more checkpoint inhibitors. Suitable checkpoint inhibitors include, but are not limited to, pembrolizurnab (Keytruda™), ipilimumab (Yervoy™), nivolumab (Opdivo™) and atezolizurnab (Tecentrig™ See e. Checkpoint Inhibitors at the CANCER RESEARCH UK website (https://www.cancerresearchuk.org/about-cancer/cancer-in-general/treatment/immunotherapy/types/checkpoint-inhibitors).

In other embodiments, the therapeutic antibodies suitable for combination with the CAR modified T cells contemplated herein, include but are not limited to, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, namatumab, naptumomab, necitumumab, nimotuzumab, nofetumomab, ocaratuzumab, ofatumumab, obinutuzumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The radioactive monoclonal antibody ibritumomab (Zevalin™) is also suitable for use in accordance with the invention.

In certain embodiments, editing a gene locus comprises using a CRISPR (Clustered Regularly Interspaced Short Palindromic Repeats)/Cas9 system, a zinc finger nuclease (ZFN), a TALEN, a MegaTAL, a meganuclease, Cpf1, homologous recombination, or a single stranded oligodeoxynucleotide (ssODN), and the like can be used.

Retroviral-based gene therapy vectors (e.g., gammaretroviral, lentiviral) are the predominant choice for CAR transduction due to the stable integration of these vectors, which results in long-term expression of the CAR. Critical vector quality attributes (e.g., titer, potency, purity) are directly determine the number of copies stably integrated into the target cells, and therefore to a large extent determine the potency of the CAR T cell product.

The present disclosure provides for a closed system method of manufacturing a population of T cells.

In certain embodiments of the disclosed method, a population of T_{N/MEM} cells is enriched by, for example, contacting the T_{N/MEM} cells with 1-100 μL CliniMACS PBS/EDTA buffer or 5-80 μL CliniMACS PBS/EDTA buffer or 10-60 μL CliniMACS PBS/EDTA buffer or 15-40 μL CliniMACS PBS/EDTA buffer.

In certain embodiments, the cells are contacted with about 15-25 μL CliniMACS PBS/EDTA buffer or about 20 μL CliniMACS PBS/EDTA buffer.

The step of contacting the cells may also preferably involve contacting the T_{NMEM} cells with microbeads.

In certain embodiments, the cells are activated by being contacted with TransAct™ for about 1 hour to about 84 hours, or about 6 hours to about 72 hours, or about 12 hours to about 60 hours, or about 18 hours to about 48 hours, or about 24 hours to about 48 hours, or from about 30 hours to about 48 hours.

Without limitation, the activation step may be performed for about 6 hours, or about 12 hours, or about 18 hours, or about 24 hours, or about 30 hours, or about 36 hours, or about 42 hours, or about 48 hours, or about 54 hours, or about 60 hours. In certain embodiments, the activation step is performed for about 42 hours.

In certain embodiments, the activation step may comprise contacting the cells with OpTmizer™ cell culture media, preferably containing plus rhIL-2 and rhIL-15 and TransAct.

In certain embodiments, following activation, the cells are transduced in the absence of protamine sulfate

In certain, non-limiting embodiments, the lentiviral vector may comprise, an EF1α promoter and anti-CD19/CD20 CAR in the pALD backbone.

Following transduction, the cells are preferably maintained for at least about 1 day, or at least about 2 days, or from about 1 day to about 14 days, or from about 1 day to about 10 days, or from about 1 day to about 7 days, or from about 2 days to about 14 days, or from about 2 days to about 10 days, or from about 2 days to about 7 days. In certain embodiments, the cells may be maintained for about 1 day, or about 2 days, or about 3 days, or about 4 days, or about 5 days, or about 6 days, or about 7 days, or about 8 days, or about 9 days, or about 10 days.

In certain embodiments, the cells are optionally cryopreserved.

Cryopreserving the T_{N/MEM} cells may be carried out by, for example, contacting the cells with Sepax C-Pro in a ratio of about 1:1 saline/HAS:CryoStor10.

In some embodiments, the maintaining step comprises adding a cell culture media 1-10 days after enriching is performed, or 2-8 days after enriching is performed, or 3-7 days after enriching is performed, or about 1 day after enriching, or about 2 days after enriching, or about 3 days after enriching, or about 5 days after enriching, or about 6 days after enriching, or about 7 days after enriching, or about 8 days after enriching, or about 9 days after enriching, or about 10 days after enriching.

The maintaining step may also comprise washing with Sepax C-Pro, optionally followed by transferring the cells to a new cell culture container, such as a cell culture bag or other cell culture vessel.

Said washing may be performed 1-10 days after enriching is performed, or 2-8 days after enriching is performed, or 3-7 days after enriching is performed, or about 1 day after enriching, or about 2 days after enriching, or about 3 days after enriching, or about 5 days after enriching, or about 6 days after enriching, or about 7 days after enriching, or about 8 days after enriching, or about 9 days after enriching, or about 10 days after enriching.

In embodiments where the cells are transferred to a new cell culture container, the cells are transferred to the new cell culture container at a cell density of about 0.1×10⁶ cells/mL to about 5.0×10⁶ cells/mL, or about 0.25×10⁶ cells/mL to about 2.0×10⁶ cells/mL, or about 0.5×10⁶ cells/mL to about 1.5×10⁶ cells/mL. Alternatively, in embodiments where the cells are transferred to a new cell culture container, the cells are transferred to the new cell culture container at a cell density of about such as about 0.1×10⁶ cells/mL, or about 0.2×10⁶ cells/mL, or about 0.3×10⁶ cells/mL, or about 0.4×10⁶ cells/mL, or about 0.5×10⁶ cells/mL, or about 0.6×10⁶ cells/mL, or about 0.7×10⁶ cells/mL, or about 0.8×10⁶ cells/mL, or about 0.9×10⁶ cells/mL, or about 1.0×10⁶ cells/mL

In an aspect, CAR molecules disclosed herein comprise four main domains: an scFv, an extracellular spacer, a transmembrane domain, and a cytoplasmic tail including costimulatory signals and the CD3z activation chain.

In an aspect, a method described herein does not comprise a CD14⁺/CD25⁺ depletion step. In a further aspect, the presently described method achieves similar or superior results as methods which comprise a CD14⁺/CD25⁺ depletion step.

The percentage of CD14+ cells in CD62L+ cells isolated with and without a CD14+/CD25+ depletion step was characterized. It was found that nearly 100% of the myeloid cells were depleted from culture after 48 hours post TransAct™ stimulation.

The extracellular domain of an anti-CD19/CD20 CAR described herein can be derived from, for example and without limitation, the antigen binding domain of Leu16 murine antibody that recognizes human CD20, and the antigen binding domain of FMC63 murine antibody that recognizes human CD19, to create a bi-specific CAR configuration. Bispecific configuration enables bivalent binding to CD19 and/or CD20 on B cells. The scFvs fragments are connected via a glycine-serine (GS) flexible linker (e.g., (G₄S)ₙ, n being 1, 2, 3, or 4 (SEQ ID NOs: 48-50 and 47, respectively); i.e., G₄S (SEQ ID NO: 48), (G₄S)₂ (SEQ ID NO: 49), (G₄S)₃ (SEQ ID NO: 50), or (G₄S)₄ (SEQ ID NO: 47)).

In an aspect, the extracellular spacer (e.g., an IgG4 extracellular spacer) serves as the hinge connecting the Leu16 scFv to the CD28 TM domain, and intracellular domains. It has been known that the hinge domain plays an important role in the function of CAR T cells. For example, the choice of the hinge region may influence CAR T cell cytokine production and activation-induced cell death (AICD) (Alabanza et al., Function of Novel Anti-CD19 Chimeric Antigen Receptors with Human Variable Regions Is Affected by Hinge and transmembrane Domains. Mol Ther J Am Soc Gene Ther (2017) 25(11):2452-65), which are directly related to the anti-tumor efficacy and the loss of CAR, respectively.

In certain embodiments, an anti-CD19/CD20 CAR disclosed herein has an amino acid sequence encoded by a nucleic acid comprising a nucleotide sequence as set forth in any one of SEQ ID NOs: 1, 3, 5, 7, 9, 11, 13, 15, 17 or 19.

In certain embodiments, an anti-CD19/CD20 CAR disclosed herein has an amino acid sequence having at least 85% sequence identity with an amino acid sequence which is encoded by a nucleic acid comprising a nucleotide sequence as set forth in any one of SEQ ID NOs: 1, 3, 5, 7, 9, 11, 13, 15, 17 or 19.

In certain embodiments, an anti-CD19/CD20 CAR disclosed herein has an amino acid sequence having at least 90% sequence identity with an amino acid sequence which is encoded by a nucleic acid comprising a nucleotide sequence as set forth in any one of SEQ ID NOs: 1, 3, 5, 7, 9, 11, 13, 15, 17 or 19.

In certain embodiments, an anti-CD19/CD20 CAR disclosed herein has an amino acid sequence having at least 95% sequence identity with an amino acid sequence which is encoded by a nucleic acid comprising a nucleotide sequence as set forth in any one of SEQ ID NOs: 1, 3, 5, 7, 9, 11, 13, 15, 17, or 19.

In certain embodiments, an anti-CD19/CD20 CAR disclosed herein has an amino acid sequence which is encoded by a nucleic acid comprising a nucleotide sequence having at least 85% sequence identity with a nucleotide sequence as set forth in any one of SEQ ID NOs: 1, 3, 5, 7, 9, 11, 13, 15, 17, or 19.

In certain embodiments, an anti-CD19/CD20 CAR disclosed herein has an amino acid sequence which is encoded by a nucleic acid comprising a nucleotide sequence having at least 90% sequence identity with a nucleotide sequence as set forth in any one of SEQ ID NOs: 1, 3, 5, 7, 9, 11, 13, 15, 17, or 19.

In certain embodiments, an anti-CD19/CD20 CAR disclosed herein has an amino acid sequence which is encoded by a nucleic acid comprising a nucleotide sequence having at least 95% sequence identity with a nucleotide sequence as set forth in any one of SEQ ID NOs: 1, 3, 5, 7, 9, 11, 13, 15, 17, and 19.

In certain embodiments, an anti-CD19/CD20 CAR disclosed herein has an amino acid sequence encoded by a nucleic acid sequence as set forth in any one of SEQ ID NOs: 1, 3, 5, 7, 9, 11, 13, 15, 17, or 19.

In certain embodiments, an anti-CD19/CD20 CAR disclosed herein has an amino acid sequence which comprises an amino acid sequence as set forth in any one of SEQ ID NOs: 2, 4, 6, 8, 10, 12, 14, 16, 18, or 20.

In certain embodiments, an anti-CD19/CD20 CAR disclosed herein has an amino acid sequence which comprises an amino acid sequence having at least 85% sequence identity with an amino acid sequence as set forth in any one of SEQ ID NOs: 2, 4, 6, 8, 10, 12, 14, 16, 18, or 20.

In certain embodiments, an anti-CD19/CD20 CAR disclosed herein has an amino acid sequence which comprises an amino acid sequence having at least 85% sequence identity with an amino acid sequence as set forth in any one of SEQ ID NOs: 2, 4, 6, 8, 10, 12, 14, 16, 18, or 20.

In certain embodiments, an anti-CD19/CD20 CAR disclosed herein has an amino acid sequence which comprises an amino acid sequence having at least 85% sequence identity with an amino acid sequence as set forth in any one of SEQ ID NOs: 2, 4, 6, 8, 10, 12, 14, 16, 18, or 20.

In certain embodiments, an anti-CD19/CD20 CAR disclosed herein has an amino acid sequence as set forth in any one of SEQ ID NOs: 2, 4, 6, 8, 10, 12, 14, 16, 18, or 20.

In certain embodiments, the hinge domain of a CAR disclosed herein has an amino acid sequence as set forth in any one of SEQ ID NOs: 21-29.

In certain embodiments, the transmembrane domain of a construct disclosed herein has an amino acid sequence comprising the sequence set forth in SEQ ID NO: 30 or 31.

In certain embodiments, the transmembrane domain of a construct disclosed herein has an amino acid sequence as set forth in SEQ ID NO: 30 or 31.

In certain embodiments, the costimulatory domain of a construct disclosed herein has an amino acid sequence comprising the sequence set forth in SEQ ID NO: 32.

In certain embodiments, the costimulatory domain of a construct disclosed herein has an amino acid sequence as set forth in SEQ ID NO: 32.

In certain embodiments, the activation domain of a construct disclosed herein has an amino acid sequence comprising the sequence set forth in SEQ ID NO: 33.

In certain embodiments, the activation domain (e.g., a CD3 zeta activation domain) of a construct disclosed herein has an amino acid sequence as set forth in SEQ ID NO: 33.

In certain embodiments, an anti-CD20 scFv comprising a light chain variable region and heavy chain variable region of a construct disclosed herein has an amino acid sequence comprising the sequences set forth in SEQ ID NOs: 34 and 35.

In certain embodiments, an anti-CD20 scFv comprising a light chain variable region and heavy chain variable region of a construct disclosed herein has an amino acid sequence as set forth in SEQ ID NOs: 34 and 35.

In certain embodiments, an anti-CD19 scFv comprising a light chain variable region and heavy chain variable region of a construct disclosed herein has an amino acid sequence comprising the sequences set forth in SEQ ID NOs: 36 and 37.

In certain embodiments, an anti-CD19 scFv comprising a light chain variable region and heavy chain variable region of a construct disclosed herein has an amino acid sequence as set forth in SEQ ID NOs: 36 and 37.

The sequences of SEQ ID NOs: 1-37 are set forth in Table 1 below:

TABLE 1

| SEQ ID NO | Nucleotide or amino acid sequence |
|---|---|
| 1 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCGAGAGCAAGTACGGA |
| | CCGCCCTGCCCCCCTTGCCCTATGTTCTGGGTGCTGGTGGTGGTCGGAGGCGTGCTG |
| | GCCTGCTACAGCCTGCTGGTCACCGTGGCCTTCATCATCTTTTGGGTGAAACGGGG |
| | CAGAAAGAAACTCCTGTATATATTCAAACAACCATTTATGAGACCAGTACAAACTA |
| | CTCAAGAGGAAGATGGCTGTAGCTGCCGATTTCCAGAAGAAGAAGAAGGAGGATG |
| | TGAACTGCGGGTGAAGTTCAGCAGAAGCGCCGACGCCCCTGCCTACCAGCAGGGC |
| | CAGAATCAGCTGTACAACGAGCTGAACCTGGGCAGAAGGGAAGAGTACGACGTCC |
| | TGGATAAGCGGAGAGGCCGGGACCCTGAGATGGGCGGCAAGCCTCGGCGGAAGA |
| | ACCCCCAGGAAGGCCTGTATAACGAACTGCAGAAAGACAAGATGGCCGAGGCCTA |
| | CAGCGAGATCGGCATGAAGGGCGAGCGGAGGCGGGGCAAGGGCCACGACGGCCT |
| | GTATCAGGGCCTGTCCACCGCCACCAAGGATACCTACGACGCCCTGCACATGCAGG |
| | CCCTGCCCCCAAGGTGA |
| 2 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITESKYGPPCPPCPMFWVLVVVGGVLACYSLLVTVAFIIFWV |
| | KRGRKKLLYIFKQPFMRPVQTTQEEDGCSCRFPEEEEGGCELRVKFSRSADAPAYQQG |
| | QNQLYNELNLGRREEYDVLDKRRGRDPEMGGKPRRKNPQEGLYNELQKDKMAEAYS |
| | EIGMKGERRRGKGHDGLYQGLSTATKDTYDALHMQALPPR |
| 3 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCACTACAACTCCTGCCC |
| | CACGGCCACCTACACCTGCGCCTACAATTGCCAGCCAGCCACTGTCCTTACGACCA |
| | GAGGCTTGCCGCCCCGCTGCGGGAGGCGCCGTCCATACCCGGGGCCTTGACTTCGC |
| | ATGCGATATGTTCTGGGTGCTGGTGGTGGTCGGAGGCGTGCTGGCCTGCTACAGCC |
| | TGCTGGTCACCGTGGCCTTCATCATCTTTTGGGTGAAACGGGGCAGAAAGAAACTC |
| | CTGTATATATTCAAACAACCATTTATGAGACCAGTACAAACTACTCAAGAGGAAGA |
| | TGGCTGTAGCTGCCGATTTCCAGAAGAAGAAGAAGGAGGATGTGAACTGCGGGTG |
| | AAGTTCAGCAGAAGCGCCGACGCCCCTGCCTACCAGCAGGGCCAGAATCAGCTGT |
| | ACAACGAGCTGAACCTGGGCAGAAGGGAAGAGTACGACGTCCTGGATAAGCGGAG |
| | AGGCCGGGACCCTGAGATGGGCGGCAAGCCTCGGCGGAAGAACCCCCAGGAAGGC |
| | CTGTATAACGAACTGCAGAAAGACAAGATGGCCGAGGCCTACAGCGAGATCGGCA |
| | TGAAGGGCGAGCGGAGGCGGGGCAAGGGCCACGACGGCCTGTATCAGGGCCTGTC |
| | CACCGCCACCAAGGATACCTACGACGCCCTGCACATGCAGGCCCTGCCCCCAAGGT |
| | GA |
| 4 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITTTTPAPRPPTPAPTIASQPLSLRPEACRPAAGGAVHTRGLDFACD |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 5 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCATTGAAGTTATGTATC |
| | CTCCTCCTTACCTAGACAATGAGAAGAGCAATGGAACCATTATCCATGTGAAAGGG |
| | AAACACCTTTGTCCAAGTCCCCTATTTCCCGGACCTTCTAAGCCCATGTTCTGGGTG |
| | CTGGTGGTGGTCGGAGGCGTGCTGGCCTGCTACAGCCTGCTGGTCACCGTGGCCTT |
| | CATCATCTTTTGGGTGAAACGGGGCAGAAAGAAACTCCTGTATATATTCAAACAAC |
| | CATTTATGAGACCAGTACAAACTACTCAAGAGGAAGATGGCTGTAGCTGCCGATTT |
| | CCAGAAGAAGAAGAAGGAGGATGTGAACTGCGGGTGAAGTTCAGCAGAAGCGCC |
| | GACGCCCCTGCCTACCAGCAGGGCCAGAATCAGCTGTACAACGAGCTGAACCTGG |
| | GCAGAAGGGAAGAGTACGACGTCCTGGATAAGCGGAGAGGCCGGGACCCTGAGAT |
| | GGGCGGCAAGCCTCGGCGGAAGAACCCCCAGGAAGGCCTGTATAACGAACTGCAG |
| | AAAGACAAGATGGCCGAGGCCTACAGCGAGATCGGCATGAAGGGCGAGCGGAGG |
| | CGGGGCAAGGGCCACGACGGCCTGTATCAGGGCCTGTCCACCGCCACCAAGGATA |
| | CCTACGACGCCCTGCACATGCAGGCCCTGCCCCCAAGGTGA |
| 6 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITIEVMYPPPYLDNEKSNGTIIHVKGKHLCPSPLFPGPSKP |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 7 | GCTAGCGCCGCCACCATGGAGACAGACACACTCCTGCTATGGGTGCTGCTGCTCTG |
| | GGTTCCAGGTTCCACAGGTGACATTGTGCTGACCCAATCTCCAGCTATCCTGTCTGC |
| | ATCTCCAGGGGAGAAGGTCACAATGACTTGCAGGGCCAGCTCAAGTGTAAATTAC |
| | ATGGACTGGTACCAGAAGAAGCCAGGATCCTCCCCCAAACCCTGGATTTATGCCAC |
| | ATCCAACCTGGCTTCTGGAGTCCCTGCTCGCTTCAGTGGCAGTGGGTCTGGGACCT |
| | CTTACTCTCTCACAATCAGCAGAGTGGAGGCTGAAGATGCTGCCACTTATTACTGC |
| | CAGCAGTGGAGTTTTAATCCACCCACGTTCGGAGGGGGGACCAAGCTGGAAATAA |
| | AAGGCAGTACTAGCGGTGGTGGCTCCGGGGGCGGTTCCGGTGGGGGCGGCAGCAG |
| | CGAGGTGCAGCTGCAGCAGTCTGGGGCTGAGCTGGTGAAGCCTGGGGCCTCAGTG |
| | AAGATGTCCTGCAAGGCTTCTGGCTACACATTTACCAGTTACAATATGCACTGGGT |
| | AAAGCAGACACCTGGACAGGGCCTGGAATGGATTGGAGCTATTTATCCAGGAAAT |
| | GGTGATACTTCCTACAATCAGAAGTTCAAAGGCAAGGCCACATTGACTGCAGACA |
| | AATCCTCCAGCACAGCCTACATGCAGCTCAGCAGCCTGACATCTGAGGACTCTGCG |
| | GACTATTACTGTGCAAGATCTAATTATTACGGTAGTAGCTACTGGTTCTTCGATGTC |
| | TGGGGCGCAGGGACCACGGTCACAGTAAGTAGCGGTGGAGGCGGCAGTGGCGGAG |
| | GTGGGAGCGGAGGGGGCGGTTCCGGTGGCGGGGGATCTGAGGTGAAGCTGCAGGA |
| | AAGCGGCCCTGGCCTGGTGGCCCCCAGCCAGAGCCTGAGCGTGACCTGCACCGTG |
| | AGCGGCGTGAGCCTGCCCGACTACGGCGTGAGCTGGATCCGGCAGCCCCCCAGGA |
| | AGGGCCTGGAATGGCTGGGCGTGATCTGGGGCAGCGAGACCACCTACTACAACAG |
| | CGCCCTGAAGAGCCGGCTGACCATCATCAAGGACAACAGCAAGAGCCAGGTGTTC |
| | CTGAAGATGAACAGCCTGCAGACCGACGACACCGCCATCTACTACTGCGCCAAGC |
| | ACTACTACTACGGCGGCAGCTACGCCATGGACTACTGGGGCCAGGGCACCAGCGT |
| | GACCGTGAGCAGCGGCAGCACCTCCGGCAGCGGCAAGCCTGGCAGCGGCGAGGGC |
| | AGCACCAAGGGCGACATCCAGATGACCCAGACCACCTCCAGCCTGAGCGCCAGCC |
| | TGGGCGACCGGGTGACCATCAGCTGCCGGGCCAGCCAGGACATCAGCAAGTACCT |
| | GAACTGGTATCAGCAGAAGCCCGACGGCACCGTCAAGCTGCTGATCTACCACACC |
| | AGCCGGCTGCACAGCGGCGTGCCCAGCCGGTTTAGCGGCAGCGGCTCCGGCACCG |
| | ACTACAGCCTGACCATCTCCAACCTGGAACAGGAAGATATCGCCACCTACTTTTGC |
| | CAGCAGGGCAACACACTGCCCTACACCTTTGGCGGCGGAACAAAGCTGGAAATCA |
| | CCACTACAACTCCTGCCCCACGGCCACCTACACCTGCGCCTACAATTGCCAGCCAG |
| | CCACTGTCCTTACGACCAGAGGCTTGCCGCCCCGCTGCGGGAGGCGCCGTCCATAC |
| | CCGGGGCCTTGACTTCGCATGCGATATTTACATTTGGGCACCCTTGGCCGGCACCT |
| | GCGGAGTTTTACTTCTGAGCTTGGTGATAACGCTGTACTGTAAACGGGGCAGAAAG |
| | AAACTCCTGTATATATTCAAACAACCATTTATGAGACCAGTACAAACTACTCAAGA |
| | GGAAGATGGCTGTAGCTGCCGATTTCCAGAAGAAGAAGAAGGAGGATGTGAACTG |
| | CGGGTGAAGTTCAGCAGAAGCGCCGACGCCCCTGCCTACCAGCAGGGCCAGAATC |
| | AGCTGTACAACGAGCTGAACCTGGGCAGAAGGGAAGAGTACGACGTCCTGGATAA |
| | GCGGAGAGGCCGGGACCCTGAGATGGGCGGCAAGCCTCGGCGGAAGAACCCCCAG |
| | GAAGGCCTGTATAACGAACTGCAGAAAGACAAGATGGCCGAGGCCTACAGCGAGA |
| | TCGGCATGAAGGGCGAGCGGAGGCGGGGCAAGGGCCACGACGGCCTGTATCAGGG |
| | CCTGTCCACCGCCACCAAGGATACCTACGACGCCCTGCACATGCAGGCCCTGCCCC |
| | CAAGGTGAGTCGAC |
| 8 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITTTTPAPRPPTPAPTIASQPLSLRPEACRPAAGGAVHTRGLDFACDIYIWA |
| | PLAGTCGVLLLSLVITLYC |
| | KRGRKKLLYIFKQPFMRPVQTTQEEDGCSCRFPEEEEGGCELRVKFSRSADAPAYQQG |
| | QNQLYNELNLGRREEYDVLDKRRGRDPEMGGKPRRKNPQEGLYNELQKDKMAEAYS |
| | EIGMKGERRRGKGHDGLYQGLSTATKDTYDALHMQALPPR |
| 9 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCTCTGGGCAAGTTCTTC |
| | TTGAGTCTAATATCAAGGTGCTGCCCACCTGGTCTACTCCTGTCCAACCGATGTTCT |
| | GGGTGCTGGTGGTGGTCGGAGGCGTGCTGGCCTGCTACAGCCTGCTGGTCACCGTG |
| | GCCTTCATCATCTTTTGGGTGAAACGGGGCAGAAAGAAACTCCTGTATATATTCAA |
| | ACAACCATTTATGAGACCAGTACAAACTACTCAAGAGGAAGATGGCTGTAGCTGC |
| | CGATTTCCAGAAGAAGAAGAAGGAGGATGTGAACTGCGGGTGAAGTTCAGCAGAA |
| | GCGCCGACGCCCCTGCCTACCAGCAGGGCCAGAATCAGCTGTACAACGAGCTGAA |
| | CCTGGGCAGAAGGGAAGAGTACGACGTCCTGGATAAGCGGAGAGGCCGGGACCCT |
| | GAGATGGGCGGCAAGCCTCGGCGGAAGAACCCCCAGGAAGGCCTGTATAACGAAC |
| | TGCAGAAAGACAAGATGGCCGAGGCCTACAGCGAGATCGGCATGAAGGGCGAGC |
| | GGAGGCGGGGCAAGGGCCACGACGGCCTGTATCAGGGCCTGTCCACCGCCACCAA |
| | GGATACCTACGACGCCCTGCACATGCAGGCCCTGCCCCCAAGGTGAGTCGAC |
| 10 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITSGQVLLESNIKVLPTWSTPVQP |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 11 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCAGCCAGCCACTGTCCT |
| | TACGACCAGAGGCTTGCCGCCCCGCTGCGGGAGGCGCCGTCCATACCCGGGGCCTT |
| | GACTTCGCATGCGATATGTTCTGGGTGCTGGTGGTGGTCGGAGGCGTGCTGGCCTG |
| | CTACAGCCTGCTGGTCACCGTGGCCTTCATCATCTTTTGGGTGAAACGGGGCAGAA |
| | AGAAACTCCTGTATATATTCAAACAACCATTTATGAGACCAGTACAAACTACTCAA |
| | GAGGAAGATGGCTGTAGCTGCCGATTTCCAGAAGAAGAAGAAGGAGGATGTGAAC |
| | TGCGGGTGAAGTTCAGCAGAAGCGCCGACGCCCCTGCCTACCAGCAGGGCCAGAA |
| | TCAGCTGTACAACGAGCTGAACCTGGGCAGAAGGGAAGAGTACGACGTCCTGGAT |
| | AAGCGGAGAGGCCGGGACCCTGAGATGGGCGGCAAGCCTCGGCGGAAGAACCCCC |
| | AGGAAGGCCTGTATAACGAACTGCAGAAAGACAAGATGGCCGAGGCCTACAGCGA |
| | GATCGGCATGAAGGGCGAGCGGAGGCGGGGCAAGGGCCACGACGGCCTGTATCAG |
| | GGCCTGTCCACCGCCACCAAGGATACCTACGACGCCCTGCACATGCAGGCCCTGCC |
| | CCCAAGGTGAGTCGAC |
| 12 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITSQPLSLRPEACRPAAGGAVHTRGLDFACD |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 13 | ATGGAGACAGACACACTCCTGCTATGGGTGCTGCTGCTCTGGGTTCCAGGTTCCAC |
| | AGGTGACATTGTGCTGACCCAATCTCCAGCTATCCTGTCTGCATCTCCAGGGGAGA |
| | AGGTCACAATGACTTGCAGGGCCAGCTCAAGTGTAAATTACATGGACTGGTACCAG |
| | AAGAAGCCAGGATCCTCCCCCAAACCCTGGATTTATGCCACATCCAACCTGGCTTC |
| | TGGAGTCCCTGCTCGCTTCAGTGGCAGTGGGTCTGGGACCTCTTACTCTCTCACAAT |
| | CAGCAGAGTGGAGGCTGAAGATGCTGCCACTTATTACTGCCAGCAGTGGAGTTTTA |
| | ATCCACCCACGTTCGGAGGGGGGACCAAGCTGGAAATAAAAGGCAGTACTAGCGG |
| | TGGTGGCTCCGGGGGCGGTTCCGGTGGGGGCGGCAGCAGCGAGGTGCAGCTGCAG |
| | CAGTCTGGGGCTGAGCTGGTGAAGCCTGGGGCCTCAGTGAAGATGTCCTGCAAGG |
| | CTTCTGGCTACACATTTACCAGTTACAATATGCACTGGGTAAAGCAGACACCTGGA |
| | CAGGGCCTGGAATGGATTGGAGCTATTTATCCAGGAAATGGTGATACTTCCTACAA |
| | TCAGAAGTTCAAAGGCAAGGCCACATTGACTGCAGACAAATCCTCCAGCACAGCC |
| | TACATGCAGCTCAGCAGCCTGACATCTGAGGACTCTGCGGACTATTACTGTGCAAG |
| | ATCTAATTATTACGGTAGTAGCTACTGGTTCTTCGATGTCTGGGGCGCAGGGACCA |
| | CGGTCACAGTAAGTAGCGGTGGAGGCGGCAGTGGCGGAGGTGGGAGCGGAGGGG |
| | GCGGTTCCGGTGGCGGGGGATCTGAGGTGAAGCTGCAGGAAAGCGGCCCTGGCCT |
| | GGTGGCCCCCAGCCAGAGCCTGAGCGTGACCTGCACCGTGAGCGGCGTGAGCCTG |
| | CCCGACTACGGCGTGAGCTGGATCCGGCAGCCCCCCAGGAAGGGCCTGGAATGGC |
| | TGGGCGTGATCTGGGGCAGCGAGACCACCTACTACAACAGCGCCCTGAAGAGCCG |
| | GCTGACCATCATCAAGGACAACAGCAAGAGCCAGGTGTTCCTGAAGATGAACAGC |
| | CTGCAGACCGACGACACCGCCATCTACTACTGCGCCAAGCACTACTACTACGGCGG |
| | CAGCTACGCCATGGACTACTGGGGCCAGGGCACCAGCGTGACCGTGAGCAGCGGC |
| | AGCACCTCCGGCAGCGGCAAGCCTGGCAGCGGCGAGGGCAGCACCAAGGGCGACA |
| | TCCAGATGACCCAGACCACCTCCAGCCTGAGCGCCAGCCTGGGCGACCGGGTGAC |
| | CATCAGCTGCCGGGCCAGCCAGGACATCAGCAAGTACCTGAACTGGTATCAGCAG |
| | AAGCCCGACGGCACCGTCAAGCTGCTGATCTACCACACCAGCCGGCTGCACAGCG |
| | GCGTGCCCAGCCGGTTTAGCGGCAGCGGCTCCGGCACCGACTACAGCCTGACCATC |
| | TCCAACCTGGAACAGGAAGATATCGCCACCTACTTTTGCCAGCAGGGCAACACACT |
| | GCCCTACACCTTTGGCGGCGGAACAAAGCTGGAAATCACCCGCCCCGCTGCGGGA |
| | GGCGCCGTCCATACCCGGGGCCTTGACTTCGCATGCGATATGTTCTGGGTGCTGGT |
| | GGTGGTCGGAGGCGTGCTGGCCTGCTACAGCCTGCTGGTCACCGTGGCCTTCATCA |
| | TCTTTTGGGTGAAACGGGGCAGAAAGAAACTCCTGTATATATTCAAACAACCATTT |
| | ATGAGACCAGTACAAACTACTCAAGAGGAAGATGGCTGTAGCTGCCGATTTCCAG |
| | AAGAAGAAGAAGGAGGATGTGAACTGCGGGTGAAGTTCAGCAGAAGCGCCGACG |
| | CCCCTGCCTACCAGCAGGGCCAGAATCAGCTGTACAACGAGCTGAACCTGGGCAG |
| | AAGGGAAGAGTACGACGTCCTGGATAAGCGGAGAGGCCGGGACCCTGAGATGGGC |
| | GGCAAGCCTCGGCGGAAGAACCCCCAGGAAGGCCTGTATAACGAACTGCAGAAAG |
| | ACAAGATGGCCGAGGCCTACAGCGAGATCGGCATGAAGGGCGAGCGGAGGCGGG |
| | GCAAGGGCCACGACGGCCTGTATCAGGGCCTGTCCACCGCCACCAAGGATACCTA |
| | CGACGCCCTGCACATGCAGGCCCTGCCCCCAAGGTGAGTCGAC |
| 14 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITRPAAGGAVHTRGLDFACDMFWVLVVVGGVLACYSLLVTVAFIIFWVK |
| | RGRKKLLYIFKQPFMRPVQTTQEEDGCSCRFPEEEEGGCELRVKFSRSADAPAYQQGQ |
| | NQLYNELNLGRREEYDVLDKRRGRDPEMGGKPRRKNPQEGLYNELQKDKMAEAYSE |
| | IGMKGERRRGKGHDGLYQGLSTATKDTYDALHMQALPPR |
| 15 | GCTAGCGCCGCCACCATGGAGACAGACACACTCCTGCTATGGGTGCTGCTGCTCTG |
| | GGTTCCAGGTTCCACAGGTGACATTGTGCTGACCCAATCTCCAGCTATCCTGTCTGC |
| | ATCTCCAGGGGAGAAGGTCACAATGACTTGCAGGGCCAGCTCAAGTGTAAATTAC |
| | ATGGACTGGTACCAGAAGAAGCCAGGATCCTCCCCCAAACCCTGGATTTATGCCAC |
| | ATCCAACCTGGCTTCTGGAGTCCCTGCTCGCTTCAGTGGCAGTGGGTCTGGGACCT |
| | CTTACTCTCTCACAATCAGCAGAGTGGAGGCTGAAGATGCTGCCACTTATTACTGC |
| | CAGCAGTGGAGTTTTAATCCACCCACGTTCGGAGGGGGGACCAAGCTGGAAATAA |
| | AAGGCAGTACTAGCGGTGGTGGCTCCGGGGGCGGTTCCGGTGGGGGCGGCAGCAG |
| | CGAGGTGCAGCTGCAGCAGTCTGGGGCTGAGCTGGTGAAGCCTGGGGCCTCAGTG |
| | AAGATGTCCTGCAAGGCTTCTGGCTACACATTTACCAGTTACAATATGCACTGGGT |
| | AAAGCAGACACCTGGACAGGGCCTGGAATGGATTGGAGCTATTTATCCAGGAAAT |
| | GGTGATACTTCCTACAATCAGAAGTTCAAAGGCAAGGCCACATTGACTGCAGACA |
| | AATCCTCCAGCACAGCCTACATGCAGCTCAGCAGCCTGACATCTGAGGACTCTGCG |
| | GACTATTACTGTGCAAGATCTAATTATTACGGTAGTAGCTACTGGTTCTTCGATGTC |
| | TGGGGCGCAGGGACCACGGTCACAGTAAGTAGCGGTGGAGGCGGCAGTGGCGGAG |
| | GTGGGAGCGGAGGGGGCGGTTCCGGTGGCGGGGGATCTGAGGTGAAGCTGCAGGA |
| | AAGCGGCCCTGGCCTGGTGGCCCCCAGCCAGAGCCTGAGCGTGACCTGCACCGTG |
| | AGCGGCGTGAGCCTGCCCGACTACGGCGTGAGCTGGATCCGGCAGCCCCCCAGGA |
| | AGGGCCTGGAATGGCTGGGCGTGATCTGGGGCAGCGAGACCACCTACTACAACAG |
| | CGCCCTGAAGAGCCGGCTGACCATCATCAAGGACAACAGCAAGAGCCAGGTGTTC |
| | CTGAAGATGAACAGCCTGCAGACCGACGACACCGCCATCTACTACTGCGCCAAGC |
| | ACTACTACTACGGCGGCAGCTACGCCATGGACTACTGGGGCCAGGGCACCAGCGT |
| | GACCGTGAGCAGCGGCAGCACCTCCGGCAGCGGCAAGCCTGGCAGCGGCGAGGGC |
| | AGCACCAAGGGCGACATCCAGATGACCCAGACCACCTCCAGCCTGAGCGCCAGCC |
| | TGGGCGACCGGGTGACCATCAGCTGCCGGGCCAGCCAGGACATCAGCAAGTACCT |
| | GAACTGGTATCAGCAGAAGCCCGACGGCACCGTCAAGCTGCTGATCTACCACACC |
| | AGCCGGCTGCACAGCGGCGTGCCCAGCCGGTTTAGCGGCAGCGGCTCCGGCACCG |
| | ACTACAGCCTGACCATCTCCAACCTGGAACAGGAAGATATCGCCACCTACTTTTGC |
| | CAGCAGGGCAACACACTGCCCTACACCTTTGGCGGCGGAACAAAGCTGGAAATCA |
| | CCGAGCCTAAAAGTTGTGATAAAACGCATACCTGTCCAATGTTCTGGGTGCTGGTG |
| | GTGGTCGGAGGCGTGCTGGCCTGCTACAGCCTGCTGGTCACCGTGGCCTTCATCAT |
| | CTTTTGGGTGAAACGGGGCAGAAAGAAACTCCTGTATATATTCAAACAACCATTTA |
| | TGAGACCAGTACAAACTACTCAAGAGGAAGATGGCTGTAGCTGCCGATTTCCAGA |
| | AGAAGAAGAAGGAGGATGTGAACTGCGGGTGAAGTTCAGCAGAAGCGCCGACGC |
| | CCCTGCCTACCAGCAGGGCCAGAATCAGCTGTACAACGAGCTGAACCTGGGCAGA |
| | AGGGAAGAGTACGACGTCCTGGATAAGCGGAGAGGCCGGGACCCTGAGATGGGCG |
| | GCAAGCCTCGGCGGAAGAACCCCCAGGAAGGCCTGTATAACGAACTGCAGAAAGA |
| | CAAGATGGCCGAGGCCTACAGCGAGATCGGCATGAAGGGCGAGCGGAGGCGGGG |
| | CAAGGGCCACGACGGCCTGTATCAGGGCCTGTCCACCGCCACCAAGGATACCTAC |
| | GACGCCCTGCACATGCAGGCCCTGCCCCCAAGGTGAGTCGAC |
| 16 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITEPKSCDKTHTCP |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 17 | GCTAGCGCCGCCACCATGGAGACAGACACACTCCTGCTATGGGTGCTGCTGCTCTG |
| | GGTTCCAGGTTCCACAGGTGACATTGTGCTGACCCAATCTCCAGCTATCCTGTCTGC |
| | ATCTCCAGGGGAGAAGGTCACAATGACTTGCAGGGCCAGCTCAAGTGTAAATTAC |
| | ATGGACTGGTACCAGAAGAAGCCAGGATCCTCCCCCAAACCCTGGATTTATGCCAC |
| | ATCCAACCTGGCTTCTGGAGTCCCTGCTCGCTTCAGTGGCAGTGGGTCTGGGACCT |
| | CTTACTCTCTCACAATCAGCAGAGTGGAGGCTGAAGATGCTGCCACTTATTACTGC |
| | CAGCAGTGGAGTTTTAATCCACCCACGTTCGGAGGGGGGACCAAGCTGGAAATAA |
| | AAGGCAGTACTAGCGGTGGTGGCTCCGGGGGCGGTTCCGGTGGGGGCGGCAGCAG |
| | CGAGGTGCAGCTGCAGCAGTCTGGGGCTGAGCTGGTGAAGCCTGGGGCCTCAGTG |
| | AAGATGTCCTGCAAGGCTTCTGGCTACACATTTACCAGTTACAATATGCACTGGGT |
| | AAAGCAGACACCTGGACAGGGCCTGGAATGGATTGGAGCTATTTATCCAGGAAAT |
| | GGTGATACTTCCTACAATCAGAAGTTCAAAGGCAAGGCCACATTGACTGCAGACA |
| | AATCCTCCAGCACAGCCTACATGCAGCTCAGCAGCCTGACATCTGAGGACTCTGCG |
| | GACTATTACTGTGCAAGATCTAATTATTACGGTAGTAGCTACTGGTTCTTCGATGTC |
| | TGGGGCGCAGGGACCACGGTCACAGTAAGTAGCGGTGGAGGCGGCAGTGGCGGAG |
| | GTGGGAGCGGAGGGGGCGGTTCCGGTGGCGGGGGATCTGAGGTGAAGCTGCAGGA |
| | AAGCGGCCCTGGCCTGGTGGCCCCCAGCCAGAGCCTGAGCGTGACCTGCACCGTG |
| | AGCGGCGTGAGCCTGCCCGACTACGGCGTGAGCTGGATCCGGCAGCCCCCCAGGA |
| | AGGGCCTGGAATGGCTGGGCGTGATCTGGGGCAGCGAGACCACCTACTACAACAG |
| | CGCCCTGAAGAGCCGGCTGACCATCATCAAGGACAACAGCAAGAGCCAGGTGTTC |
| | CTGAAGATGAACAGCCTGCAGACCGACGACACCGCCATCTACTACTGCGCCAAGC |
| | ACTACTACTACGGCGGCAGCTACGCCATGGACTACTGGGGCCAGGGCACCAGCGT |
| | GACCGTGAGCAGCGGCAGCACCTCCGGCAGCGGCAAGCCTGGCAGCGGCGAGGGC |
| | AGCACCAAGGGCGACATCCAGATGACCCAGACCACCTCCAGCCTGAGCGCCAGCC |
| | TGGGCGACCGGGTGACCATCAGCTGCCGGGCCAGCCAGGACATCAGCAAGTACCT |
| | GAACTGGTATCAGCAGAAGCCCGACGGCACCGTCAAGCTGCTGATCTACCACACC |
| | AGCCGGCTGCACAGCGGCGTGCCCAGCCGGTTTAGCGGCAGCGGCTCCGGCACCG |
| | ACTACAGCCTGACCATCTCCAACCTGGAACAGGAAGATATCGCCACCTACTTTTGC |
| | CAGCAGGGCAACACACTGCCCTACACCTTTGGCGGCGGAACAAAGCTGGAAATCA |
| | CCGAGCCAAAGTCATGTGATACACCTCCTCCCTGTCCCATGTTCTGGGTGCTGGTG |
| | GTGGTCGGAGGCGTGCTGGCCTGCTACAGCCTGCTGGTCACCGTGGCCTTCATCAT |
| | CTTTTGGGTGAAACGGGGCAGAAAGAAACTCCTGTATATATTCAAACAACCATTTA |
| | TGAGACCAGTACAAACTACTCAAGAGGAAGATGGCTGTAGCTGCCGATTTCCAGA |
| | AGAAGAAGAAGGAGGATGTGAACTGCGGGTGAAGTTCAGCAGAAGCGCCGACGC |
| | CCCTGCCTACCAGCAGGGCCAGAATCAGCTGTACAACGAGCTGAACCTGGGCAGA |
| | AGGGAAGAGTACGACGTCCTGGATAAGCGGAGAGGCCGGGACCCTGAGATGGGCG |
| | GCAAGCCTCGGCGGAAGAACCCCCAGGAAGGCCTGTATAACGAACTGCAGAAAGA |
| | CAAGATGGCCGAGGCCTACAGCGAGATCGGCATGAAGGGCGAGCGGAGGCGGGG |
| | CAAGGGCCACGACGGCCTGTATCAGGGCCTGTCCACCGCCACCAAGGATACCTAC |
| | GACGCCCTGCACATGCAGGCCCTGCCCCCAAGGTGAGTCGAC |
| 18 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITEPKSCDTPPPCP |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 19 | GCTAGCGCCGCCACCATGGAGACAGACACACTCCTGCTATGGGTGCTGCTGCTCTG |
| | GGTTCCAGGTTCCACAGGTGACATTGTGCTGACCCAATCTCCAGCTATCCTGTCTGC |
| | ATCTCCAGGGGAGAAGGTCACAATGACTTGCAGGGCCAGCTCAAGTGTAAATTAC |
| | ATGGACTGGTACCAGAAGAAGCCAGGATCCTCCCCCAAACCCTGGATTTATGCCAC |
| | ATCCAACCTGGCTTCTGGAGTCCCTGCTCGCTTCAGTGGCAGTGGGTCTGGGACCT |
| | CTTACTCTCTCACAATCAGCAGAGTGGAGGCTGAAGATGCTGCCACTTATTACTGC |
| | CAGCAGTGGAGTTTTAATCCACCCACGTTCGGAGGGGGGACCAAGCTGGAAATAA |
| | AAGGCAGTACTAGCGGTGGTGGCTCCGGGGGCGGTTCCGGTGGGGGCGGCAGCAG |
| | CGAGGTGCAGCTGCAGCAGTCTGGGGCTGAGCTGGTGAAGCCTGGGGCCTCAGTG |
| | AAGATGTCCTGCAAGGCTTCTGGCTACACATTTACCAGTTACAATATGCACTGGGT |
| | AAAGCAGACACCTGGACAGGGCCTGGAATGGATTGGAGCTATTTATCCAGGAAAT |
| | GGTGATACTTCCTACAATCAGAAGTTCAAAGGCAAGGCCACATTGACTGCAGACA |
| | AATCCTCCAGCACAGCCTACATGCAGCTCAGCAGCCTGACATCTGAGGACTCTGCG |
| | GACTATTACTGTGCAAGATCTAATTATTACGGTAGTAGCTACTGGTTCTTCGATGTC |
| | TGGGGCGCAGGGACCACGGTCACAGTAAGTAGCGGTGGAGGCGGCAGTGGCGGAG |
| | GTGGGAGCGGAGGGGGCGGTTCCGGTGGCGGGGGATCTGAGGTGAAGCTGCAGGA |
| | AAGCGGCCCTGGCCTGGTGGCCCCCAGCCAGAGCCTGAGCGTGACCTGCACCGTG |
| | AGCGGCGTGAGCCTGCCCGACTACGGCGTGAGCTGGATCCGGCAGCCCCCCAGGA |
| | AGGGCCTGGAATGGCTGGGCGTGATCTGGGGCAGCGAGACCACCTACTACAACAG |
| | CGCCCTGAAGAGCCGGCTGACCATCATCAAGGACAACAGCAAGAGCCAGGTGTTC |
| | CTGAAGATGAACAGCCTGCAGACCGACGACACCGCCATCTACTACTGCGCCAAGC |
| | ACTACTACTACGGCGGCAGCTACGCCATGGACTACTGGGGCCAGGGCACCAGCGT |
| | GACCGTGAGCAGCGGCAGCACCTCCGGCAGCGGCAAGCCTGGCAGCGGCGAGGGC |
| | AGCACCAAGGGCGACATCCAGATGACCCAGACCACCTCCAGCCTGAGCGCCAGCC |
| | TGGGCGACCGGGTGACCATCAGCTGCCGGGCCAGCCAGGACATCAGCAAGTACCT |
| | GAACTGGTATCAGCAGAAGCCCGACGGCACCGTCAAGCTGCTGATCTACCACACC |
| | AGCCGGCTGCACAGCGGCGTGCCCAGCCGGTTTAGCGGCAGCGGCTCCGGCACCG |
| | ACTACAGCCTGACCATCTCCAACCTGGAACAGGAAGATATCGCCACCTACTTTTGC |
| | CAGCAGGGCAACACACTGCCCTACACCTTTGGCGGCGGAACAAAGCTGGAAATCA |
| | CCGTACCCTGTCGAGTACCGCCTCCGCCCCCATGCTGCCACCCGATGTTCTGGGTGC |
| | TGGTGGTGGTCGGAGGCGTGCTGGCCTGCTACAGCCTGCTGGTCACCGTGGCCTTC |
| | ATCATCTTTTGGGTGAAACGGGGCAGAAAGAAACTCCTGTATATATTCAAACAACC |
| | ATTTATGAGACCAGTACAAACTACTCAAGAGGAAGATGGCTGTAGCTGCCGATTTC |
| | CAGAAGAAGAAGAAGGAGGATGTGAACTGCGGGTGAAGTTCAGCAGAAGCGCCG |
| | ACGCCCCTGCCTACCAGCAGGGCCAGAATCAGCTGTACAACGAGCTGAACCTGGG |
| | CAGAAGGGAAGAGTACGACGTCCTGGATAAGCGGAGAGGCCGGGACCCTGAGATG |
| | GGCGGCAAGCCTCGGCGGAAGAACCCCCAGGAAGGCCTGTATAACGAACTGCAGA |
| | AAGACAAGATGGCCGAGGCCTACAGCGAGATCGGCATGAAGGGCGAGCGGAGGC |
| | GGGGCAAGGGCCACGACGGCCTGTATCAGGGCCTGTCCACCGCCACCAAGGATAC |
| | CTACGACGCCCTGCACATGCAGGCCCTGCCCCCAAGGTGAGTCGAC |
| 20 | METDTLLLWVLLLWVPGSTGDIVLTQSPAILSASPGEKVTMTCRASSSVNYMDWYQK |
| | KPGSSPKPWIYATSNLASGVPARFSGSGSGTSYSLTISRVEAEDAATYYCQQWSFNPPT |
| | FGGGTKLEIKGSTSGGGSGGGSGGGGSSEVQLQQSGAELVKPGASVKMSCKASGYTFT |
| | SYNMHWVKQTPGQGLEWIGAIYPGNGDTSYNQKFKGKATLTADKSSSTAYMQLSSLT |
| | SEDSADYYCARSNYYGSSYWFFDVWGAGTTVTVSSGGGGSGGGGSGGGGSGGGGSE |
| | VKLQESGPGLVAPSQSLSVTCTVSGVSLPDYGVSWIRQPPRKGLEWLGVIWGSETTYY |
| | NSALKSRLTIIKDNSKSQVFLKMNSLQTDDTAIYYCAKHYYYGGSYAMDYWGQGTSV |
| | TVSSGSTSGSGKPGSGEGSTKGDIQMTQTTSSLSASLGDRVTISCRASQDISKYLNWYQ |
| | QKPDGTVKLLIYHTSRLHSGVPSRFSGSGSGTDYSLTISNLEQEDIATYFCQQGNTLPYT |
| | FGGGTKLEITVPCRVPPPPPCCHP |
| | MFWVLVVVGGVLACYSLLVTVAFIIFWVKRGRKKLLYIFKQPFMRPVQTTQEEDGCSC |
| | RFPEEEEGGCELRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTY |
| | DALHMQALPPR |
| 21 | ESKYGPPCPPCP |
| 22 | TTTPAPRPPTPAPTIASQPLSLRPEACRPAAGGAVHTRGLDFACD |
| 23 | IEVMYPPPYLDNEKSNGTIIHVKGKHLCPSPLFPGPSKP |
| 24 | SGQVLLESNIKVLPTWSTPVQP |
| 25 | SQPLSLRPEACRPAAGGAVHTRGLDFACD |
| 26 | RPAAGGAVHTRGLDFACD |
| 27 | EPKSCDKTHTCP |
| 28 | EPKSCDTPPPCP |
| 29 | VPCRVPPPPPCCHP |
| 30 | FWVLVVVGGVLACYSLLVTVAFIIFWV |
| 31 | IYIWAPLAGTCGVLLLSLVITLYC |
| 32 | KRGRKKLLYIFKQPFMRPVQTTQEEDGCSCRFPEEEEGGCEL |
| 33 | RVKFSRSADAPAYQQGQNQLYNELNLGRREEYDVLDKRRGRDPEMGGKPRRKNPQE |
| | GLYNELQKDKMAEAYSEIGMKGERRRGKGHDGLYQGLSTATKDTYDALHMQALPPR |
| 34 | DIVLTQSPAILSASPGEKVTMTCRASSSVNYMDWYQKKPGSSPKPWIYATSNLASGVP |
| | ARFSGSGSGTSYSLTISRVEAEDAATYYCQQWSFNPPTFGGGTKLEIK |
| 35 | EVQLQQSGAELVKPGASVKMSCKASGYTFTSYNMHWVKQTPGQGLEWIGAIYPGNG |
| | DTSYNQKFKGKATLTADKSSSTAYMQLSSLTSEDSADYYCARSNYYGSSYWFFDVWG |
| | AGTTVTVSS |
| 36 | EVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTS |
| | VTVSS |
| 37 | DIQMTQTTSSLSASLGDRVTISCRASQDISKYLNWYQQKPDGTVKLLIYHTSRLHSGVP |
| | SRFSGSGSGTDYSLTISNLEQEDIATYFCQQGNTLPYTFGGGTKLEIT |

### EXAMPLES

### Example 1: Construction of Chimeric Antigen Receptor (CAR) Expressing Vectors

Exemplary tandem targeting constructs for anti-CD19/CD20 CAR as described herein were generated. The amino acid sequences of these exemplary constructs are set forth in SEQ ID NOs: 2, 4, 6, 8, 10, 12, 14, 16, 18 and 20. The constructs were generated by the following protocol.

For example, the components of an anti-CD19/CD20 CAR having the amino acid sequence of SEQ ID NO: 2 is illustrated below:

```
METDTLLLWV LLLWVPGSTG DIVLTQSPAI LSASPGEKVT MTCRASSSVN YMDWYQKKPG
60
|--leader sequence--> |------------------------------------------
SSPKPWIYAT SNLASGVPAR FSGSGSGTSY SLTISRVEAE DAATYYCQQW SFNPPTFGGG
          120
----------------------anti-CD20 scFv VL--------------------------
TKLEIKGSTS GGGSGGGSGG GGSSEVQLQQ SGAELVKPGA SVKMSCKASG YTFTSYNMHW
          180
-----> |--------------------------------------
VKQTPGQGLE WIGAIYPGNG DTSYNQKFKG KATLTADKSS STAYMQLSSL TSEDSADYYC
          240
-----------------------anti-CD20 scFv VH-------------------------
ARSNYYGSSY WFFDVWGAGT TVTVSSGGGG SGGGGSGGGG SGGGGSEVKL QESGPGLVAP
          300
--------------------------->|-----GS linker------>|--------------
SQSLSVTCTV SGVSLPDYGV SWIRQPPRKG LEWLGVIWGS ETTYYNSALK SRLTIIKDNS
          360
-----------------------anti-CD19 scFv VH-------------------------
KSQVFLKMNS LQTDDTAIYY CAKHYYYGGS YAMDYWGQGT SVTVSSGSTS GSGKPGSGEG
          420
------------------------------------------------->
STKGDIQMTQ TTSSLSASLG DRVTISCRAS QDISKYLNWY QQKPDGTVKL LIYHTSRLHS
          480
        |------------------anti-CD19 scFv VL-------------------------
GVPSRFSGSG SGTDYSLTIS NLEQEDIATY FCQQGNTLPY TFGGGTKLEI TESKYGPPCP
          540
--------------------------------------> |-spacer-
PCPMFWVLVV VGGVLACYSL LVTVAFIIFW VKRGRKKLLY IFKQPFMRPV QTTQEEDGCS
          600
-->|--------------TM------------->|-------------------4-1BB------
CRFPEEEEGG CELRVKFSRS ADAPAYQQGQ NQLYNELNLG RREEYDVLDK RRGRDPEMGG
          660
--------------->|------------------------------------------------
KPRRKNPQEG LYNELQKDKM AEAYSEIGMK GERRRGKGHD GLYQGLSTAT KDTYDALHMQ
          720
-----------------------------CD3 zeta---------------------------
ALPPR 725
---->
```

In each case, a full length insert spanning an EF1α promoter was chemically synthesized by linking, in frame, the following encoding nucleic acid sequences: (i) an anti-CD20 single-chain variable fragment (scFv) derived from Leu16 monoclonal antibody and an anti-CD19 single-chain variable fragment (scFv) derived from FMC63 monoclonal antibody linked in sequence by a flexible interchain linker; (ii) the IgG4 hinge, (iii) the CD28 transmembrane domain, (iv) cytoplasmic domains of 4-1BB, and (v) CD3 zeta.

A leader sequence derived from murine IgG kappa leader sequence was included in all constructs. Full length CAR constructs sequences were cloned into the BstBI-SalI restriction sites of the third generation lentiviral plasmid backbone of pALD-Lenti EGFP-K (Aldevron, Fargo, ND), replacing the sFFV promoter and EGFP gene.

Lentiviral vector (LV) containing supernatants were generated by transient transfection of HEK 293T cells.). Harvested pelleted lentiviral supernatants were stored at −80° C.

CD62L+ T cells were isolated from healthy donor whole blood obtained from Stem Cell Technology. Isolated cells were stimulated with CD3/CD28 TransAct™, a Human T cell expander CD3/CD28 (Miltenyi Biotec), and cultured in OpTmizer™ media (ThermoFisher) with 1× GlutaMAX™ (ThermoFisher), Interleukin-2 (IL-2) and IL-15.

T cells were lentivirally transduced 38-42 hours later at a multiplicity of infection of 5 After 6 or 7 days in TransAct-containing media, the cells were centrifuged, and the media is replaced by fresh media without TransAct. T cells transduced with the lentiviral vector encoding the anti-CD19/CD20 CAR were harvested and cryopreserved once a sufficient amount was obtained for administration.

The novel anti-CD19/CD20 CARs having the amino acid sequences of SEQ ID NOs: 2, 4, 6, 8, 10, 12, 14, 16, 18 and 20 contained the tandem scFv in their extracellular domain. T cells expressing these CARs specifically recognized both CD19+ and CD20+ target cells and carried out functions including degranulation, cytokine release, and proliferation. These CARs were compared with hinge and transmembrane domains derived from different human B cells (IgG) and human T cell molecules to evaluate the potency of transduced CAR T cells to eliminate established tumors in mice and human.

### Example 2: CAR Detection—CAR Expression is Stable Post-Transduction

CD62L⁺ naïve/memory T cells (T_{N/MEM}) were isolated from healthy donor Leukopaks without performing depletion of CD14+ and C25+ cells. Enrichment of CD62L⁺ cells was performed using magnetic microbeads from Miltenyi Biotec. T_{N/MEM} cells (1.5×10⁶/mL) were activated with GMP-grade TransAct™ Human T cell expander CD3/CD28 (Miltenyi Biotec) at a 1:35 dilution and T cells were then cultured in CTS OpTmizer™ media supplemented with GMP-grade recombinant human IL-2 (50 U/mL, Miltenyi Biotec) and GMP-grade recombinant IL-15 (0.5 ng/mL, Miltenyi Biotec). Two days after activation, 1×10⁶/mL T_{N/MEM} cells were transduced with vector at a titration of multiplicity of infection (MOI) from 0.5 to 20 and GMP-grade LentiBOOST™ (Sirion Biotech) at a dilution of 1:100. T_{N/MEM} cells were incubated with lentivirus for 4 hours and then diluted to a final concentration of 0.5×10⁶ cells/mL with CTS OpTmizer™ media supplemented with IL-2 (50 U/mL) and IL-15 (0.5 ng/mL) and cultured at 37° C. with fresh IL-2 and IL-15 were added to the media every Monday, Wednesday, and Friday. TransAct was removed from cell culture 7 days after activation.

Surface transgene expression analysis was performed using flow cytometry 7 day (immediately after removing TransAct™), 10 days, and 12 days after activation. Cells were washed using Flow Cytometry Staining Buffer (BD) and stained with antibodies for 30 minutes at 4° C. Stained cells were washed using Staining Buffer (BD) and resuspended at 1×10⁶ cells/mL for flow cytometry analysis. Single cells were identified based on forward and side scatter distributions and viable cells were identified based on live/dead cell stain. Transgene positive cells were identified by surface staining with anti-mouse IgG F(ab)2-Biotin, which stains light chain of anti-CD19/CD20 CAR.

### Example 3: Vector Copy Number Linearly Correlates with Surface CAR Expression

T cells were harvested on Day 12. Vector copy number analysis was performed on transduced cells using droplet digital PCR (ddPCR). The total number of copies of the vector per transduced cell was determined by ddPCR. Genomic DNA was extracted from transduced T cells using the Qiagen DNeasy™ Blood and Tissue Kit (Qiagen) following manufacturer's protocol. The concentration of genomic DNA samples were measured using NanoDrop™ One/One^{C} Microvolume UV-Vis Spectrophotometer (Thermo Fisher Scientific). Probes and forward and reverse primers for ddPCR were synthesized by Integrated DNA Technologies (IDT). Primers and a FAM-labeled probe specific to the Leu16 scFv and FMC63 scFv element of the lentiviral vectors were used for transgene detection (Table 2, Set A). As a reference gene, syndecan-4 (NCBI Gene ID: 6385; HGNC:10661) with a HEX-labeled probe was used (Table 2, Set B).

TABLE 2
Primer and probe sequences

| | | Sequences (5′-3′) of primers and |
|---|---|---|
| | Primers/probes | probe used for ddPCR |
| Set A: | Set A: Leu16 scFv | Forward: CTTATTACTGCCAGCAGTGGAG |
| CD19/20 | (Target) | (SEQ ID NO: 38) |
| CAR | | Reverse: CAGGACATCTTCACTGAGGC |
| | | (SEQ ID NO: 39) |
| | FAM/Iowa Black FQ | Probe: CGGAGCCACCACCGCTAGTACTG |
| | | (SEQ ID NO: 40) |
| Set B: | Set B: FMC63 scFv | Forward: CGACATCCAGATGACCCAG |
| CD19/20 | (Target) | (SEQ ID NO: 41) |
| CAR | | Reverse: GTTCCAGGTTGGAGATGGTC |
| | | (SEQ ID NO: 42) |
| | HEX/ZEN/Iowa Black | Probe: |
| | FQ | CACCGTCAAGCTGCTGATCTACCACACCA |
| | | (SEQ ID NO: 43) |
| Set D: | Set D: SDC4 (Reference) | Forward: CAGGGTCTGGGAGCCAAGT |
| Reference | | (SEQ ID NO: 44) |
| | | Reverse: GCACAGTGCTGGACATTGACA |
| | | (SEQ ID NO: 45) |
| | HEX/ZEN/Iowa Black | Probe: |
| | FQ | CCCACCGAACCCAAGAAACTAGAGGAGAAT |
| | | (SEQ ID NO: 46) |

PCR reaction mix was prepared using 10 μL of 2× ddPCR supermix for probes (No dUTP) (BioRad), primers at final concentrations of 500 nM, probes at final concentrations of 250 nM. Prior to ddPCR, 15-40 ng of genomic DNA was linearized using Bsp1407I (Thermo Fisher Scientific) for 30 mins at 37° C. Afterwards, 20 μL of PCR mixes and 70 μL of droplet generation oil (Bio-Rad) were loaded into the DG8 Cartridges with gaskets, yielding a final volume of 40 μL product from the QX200™ Droplet Generator (Bio-Rad). The droplet emulsion was transferred to a 96-well PCR plate and DNAs were amplified at following conditions in a C1000 Touch Thermal Cycler (Bio-Rad): denaturation at 95° C. for 10 min; 40 cycles at 94° C. for 30 s, 60° C. for 1 min and 98° C. for 10 min; hold at 12° C. Data acquisition was done within 24 hours in the QX200™ Droplet Reader (Bio-Rad) and analysis was performed with the QuantaSoft™ Software (Bio-Rad). No template controls (NTC) were used to monitor contaminations of reagents and the formation of primer dimers. Untransduced T cells (UTD) from the same donor was used as negative control to evaluate off-target amplification.

### Example 4: Cytotoxic T Lymphocyte Assay

To evaluate the potency of anti-CD19/CD20 CAR transduced T_{N/MEM} cells, transduced T cells were incubated with target cells with and without CD19 and CD20 antigen for 24 hours to quantify antigen specific CAR-T cell cytotoxicity. CD19 and CD20 negative target cell lines were generated from parental wildtype Raji cells (CD19+ and CD20+) purchased from ATCC. CD19 and C20 antigens were knocked out using CRISPR/Cas9, expanded from single cell clones, and sequence verified using NGS.

Anti-CD19/CD20 CAR T_{N/MEM} cells were incubated with 10,000 luciferase expressing Raji cells at an effector to target (E:T) ratio of 10:1 (10 CAR positive T cells to 1 Raji cell) and titrated serially down by a factor to 2 for 9 dilutions in a 96 well plate. The last well served as a target cell only control. Cells were incubated for 18 hours in a 37° C., 5% CO₂ incubator, after which 120 μL of supernatant containing secreted cytokines was removed and stored at −20° C. for further analysis (14.2.4). 20 μL Bio-Glo Luciferase reagent (Promega) was added to the remainder of the cell suspension and incubated at room temperature, protected from light, for 15 minutes. Luminescence was read on the Varioskan™ (Thermo Fisher).

CTL data was collected across 3 donors. T cell effector function was quantified based on target cell viability after 18 hours of co-culture. Anti-CD19/CD20 CAR T cells demonstrated killing of Raji WT, Raji CD19 KO, and Raji CD20 KO cells with >60% CTL observed at E:T of 1:1.

### Example 5: Cytokine Secretion Assay

To measure IFN-γ and IL-2 secreted in cell-mediated cytotoxicity assays, T cells and target cells were co-cultured at varying ratios of effector to target (E:T) in the absence of exogenous cytokines and incubated for 18 hours at 37° C. and 5% CO₂. After incubation, supernatants were harvested, diluted 10×, and analyzed for secretion of IL-2 and IFN-γ using R&D Systems ELISA kits (D2050 and DIF50C, respectively) following manufacturer's protocol. In both assays, 100 μL of Assay Diluent and 100 μL of either cytokine standard or sample were added into each well and incubated at room temperature for 2 hours. Samples were washed and incubated with the anti-IL-2 or IFN-γ antibody conjugates at room temperature for 2 hours. After an additional washing step, substrate solution was added and incubated at room temperature, protected from light, for 20 minutes. Finally, stop solution was added and optical density was read at 450 nm and 540 nm. Values read at 540 nm were subtracted from the values at 450 nm which corrects for optical imperfections of the plate. Quantification of cytokine secretion is based on the optical density from the reaction of enzyme-linked polyclonal antibody in contact with a substrate and detected on a spectrophotometer. All samples were performed in triplicates, unless otherwise noted.

Consistent with observations in the CTL assay, anti-CD19/CD20 CAR T_{N/MEM} cells secreted IL-2 and IFN-γ when co-cultured with either Raji WT, Raji CD19 KO, or Raji CD20 KO cell lines. Peak secreted IL-2 and IFN-γ was observed at E:T ratio of 10:1.

### Example 6: Manufacture

Approximately 14-28 days prior to infusion of transduced cells, patients will undergo leukapheresis to collect white blood cells (Day 0). The following day (Day 1) or up to 72 hours after, the leukapheresis product will be enriched for CD62L+ cells. The CD62L+ cells are called naïve memory T cells, or T_{N/MEM}. Within 2 hours of CD62L+ enrichment, T_{N/MEM} cells will be stimulated with clinical-grade CD3/CD28 TransAct™ (Miltenyi Biotec) and fed with 50 U/mL interleukin (IL)-2 and 0.5 ng/mL IL-15. On M-Day 3, cells will be transduced with the lentivirus vector CC314B. Transduced cells (henceforth referred to as CART19/20 cells) will be expanded ex vivo, with the addition of IL-2 and IL-15 (50 U/mL and 0.5 ng/mL final concentration, respectively) every other day. Additional culture media will be added and total cell culture volume will be expanded as necessary to maintain a cell density of 0.3-0.6×106 cells/mL. TransAct™ will be removed on M-Day 7, and cell expansion will continue until sufficient cohort-specific CAR+ cell numbers have been generated, with manufacturing estimated to finish on M-Day 12.

For example, in one embodiment, TransAct™ will be removed on Day 7, and cell expansion will continue for 5 days or more after transduction until sufficient viable CAR+ cell numbers (e.g., 2×10⁸ total cells) have been generated, with manufacturing estimated to finish on Day 12-14.

### Example 7: Comparison of Transduction Enhancers

Generation of CAR-T cells through lentiviral transduction can be improved by adding transduction enhancers such as polybrene (RetroNectin, Takara Bio), protamine sulfate (Fresenius Kabi), LentiBoost™ (Sirion Biotech, Germany), Vectofusin-1 (Miltenyi Biotech, Germany), and poloxamer.

Transduction efficiency using no transduction enhancer, protamine sulfate, and LentiBoost™ were compared in this study. CD62L⁺ naïve/memory T cells (T_{N/MEM}) were isolated from healthy donors and transduced in a composition comprising transduction enhancer, IL-2, IL-15, TransAct™ and a lentivirus comprising a nucleic acid encoding the anti-CD19/CD20 CAR in pALD backbone (Lentigen). The cells were transduced at a concentration of 1×10⁶ cells/mL at multiplicity of infection (MOI) of 0.5 and 5 based on manufacture's titer. Cells were incubated with virus, IL-2 and/or IL-15 with or without transduction enhancers for 6 hours prior to equal volume of media addition.

At Day 12 after initial activation of T cells, CAR expression, vector copy number and potency assays were performed. At low MOI, T cells transduced using LentiBoost™ yields higher transduction efficiency than using protamine sulfate or without addition of any transduction enhancer.

### Example 8: Production of IMPT-314 (pALD-Leu16-FMC63-CD28_BBz)

IMPT-314 (pALD-Leu16-FMC63-BBz) is produced by obtaining autologous T cells which are then transduced with lentiviral vector to express the anti-CD19/CD20 CAR.

Autologous T cells are collected by leukapheresis. A CD14+/CD25+ depletion step is not performed. The production of IMPT-314 from subject apheresis material is a continuous process without any hold steps between production of drug substance and drug product. The IMPT-314 drug product is CART19/20 cells formulated in a 1:1 mixture of CryoStor CS-10 and Plasmalyte/5% human serum albumin (HSA), for a final concentration of 5% DMSO and 2.5% plasmalyte. IMPT-314 drug product will include both transduced (CAR+) and untransduced (CAR) cells.

CD14+/CD25+ depletion step: Prior protocols used in the art required and included a CD14+/CD25+ cells negative selection (i.e., depletion) step. At the beginning of the protocol, the leukapheresis sample was analyzed for CBC and by flow cytometry for CD3+, CD14+, CD25+, and CD62L+ cells. This next step in the process was dependent on the flow cytometry results. If % of CD62L+ cells that were also either CD25+ or CD14+ was >5% then a depletion step was performed by removal of CD14+/CD25+ cells using the ClinMACS column. However, if the % CD14+ and/or CD25+ within CD62L+ population is <5% the protocol permits the CD14+/CD25+ depletion step to be bypassed and move directly to CD62L enrichment using the ClinMACS column.

TABLE 3
Comparison of Previous Manufacturing Method and Method of the Present Invention

| | Previous | Method of the | |
|---|---|---|---|
| | Manufacturing | Present | |
| Process step | Methods | Disclosure | Observation |
| CD62L+ enrichment | Addition of Human | CliniMACS Plus | No difference has been observed in the |
| step | Immune globulin - IgG | enrichment | level of enrichment with and without |
| | (Gammagard, Takeda) at | | IgG addition. |
| | 0.28% | | |
| CD14+/CD25+ | Performed if % CD14+ | Not performed | No difference in transduction efficiency |
| depletion step | and/or CD25+ within | | has been observed between the |
| | CD62L+ population | | manufacturing process with and without |
| | is >5% | | the depletion step. |
| Cell washing (buffer | Manual Centrifugation | Sepax C-Pro | Use of an automated, closed system to |
| exchange) at Day 0, | | | reduce contamination risks and |
| Day 7 and Day 12 | | | potential human errors. |
| Cell activation prior | Duration of 32-48 hours | Duration of 38-46 | Reduction of the activation window to |
| to transduction | | hours (target 42 | improve process consistency. |
| | | hours) | |
| Transduction | Protamine sulfate, as a | No enhancer added | No significant or consistent |
| | transduction enhancer. | | improvement in transduction efficiency |
| | | | has been observed with or without |
| | | | protamine sulfate. |
| Drug product | Cryostor CS5 | Cryostor CS10 and | Use of automated closed system (Sepax |
| formulation | | Plasmalyte/5% | C-Pro) for the final wash step prior to |
| | | HSA (1:1 ratio) | formulation: cells are resuspended in |
| | | | Plasmalyte at 5% HSA. |
| | | | The formulation maintains a final 5% |
| | | | DMSO concentration, which is critical |
| | | | for cryopreservation. |

Abbreviations:
DP = drug product;
HSA = human serum albumin;
IgG = immunoglobulin G;
MOI = multiplicity of infection;
US = United States

### Example 9: Vector Drug Substance

In accordance with the present disclosure, the anti-CD19/CD20 CAR utilizes a lentiviral backbone, the pALD, Aldevron's (Fargo, North Dakota) Lenti expression plasmid, to replace the anti-CD19/CD20 CARs viral cloning plasmid and moved to an alternative manufacturer.

Both the anti-CD19/CD20 CAR transgene and the EF1a promoter are incorporated into the pALD plasmid. The plasmid containing the insert is 8,397 kb in size and contains lentiviral regulatory elements. A map of the pALD_CD19/CD20 CAR plasmid is shown in FIG. 7.

The antibiotic selectable marker in pALD is Kanamycin. Aminoglycosides such as kanamycin and neomycin are currently preferred since they are rarely used in the clinic.

The WPRE is a common feature of contemporary retroviral vectors that can improve production and potentially gene expression in target cells. This sequence is driven from the woodchuck hepatitis virus post-transcriptional regulatory element and prevents poly(A) site readthrough, to promote RNA processing and maturation, while increasing nuclear export of RNA. Safety concerns have been raised regarding the potential oncogenic activity of the truncated woodchuck hepatitis virus X protein encoded in this element. Therefore, in accordance with the invention, this capability was abrogated by mutating the WPRE ORF translation to prevent potential expression of the X protein start codon from ATG to TTG. It was found that the native form or mutated derivatives of WPRE function equivalently.

### Lentiviral Vector Components

The third generation pALD lentiviral vector is a replication-incompetent and self-inactivating vector, due to the number of essential genes that have been deleted. It includes an additional number of safety features; an altered 3′ long terminal repeat (LTR) renders the vector “self-inactivating” to prevent integrated genes from being repackaged and a heterologous coat protein (e.g., VSV-G) is used in place of the native HIV-1 envelope protein. Key components of the pALD vector are described in Section below.

CMV promoter: The cytomegalovirus (CMV) promoter replaces the U3 LTR in SIN vectors and drives transcription of viral ribonucleic acid (RNA) in packaging cells. This RNA is then packaged into live virus.

5′ LTR-AU3: This is a deleted version of the HIV-1 5′ long terminal repeat. The LTRs carry both promoter and polyadenylation function. In the pALD plasmid, 5′ LTR-ΔU3 is deleted for safety. This does not affect the production of viral RNA during packaging because the promoter function is supplemented by the CMV promoter engineered upstream of 5′LTR-ΔU3 LTR.

Ψ: HIV-1 packaging signal required for the packaging of viral RNA into virus.

RRE: HIV-1 Rev Response Element (RRE). RRE permits the nuclear export of viral RNA by the viral Rev protein during viral packaging.

cPPT: HIV-1 Central Polypurine Tract (cPPT). cPPT creates a “DNA flap” that increases nuclear import of the viral genome during target cell infection. This improves vector integration into the host genome, resulting in higher transduction efficiency.

Incorporation of a cPPT and a posttranscriptional regulatory element (PRE) into lentivirus vectors provides increased transduction efficiency and transgene expression.

Hybrid EF1a/HTLV1 promoter: The hEF1a-HTLV promoter is a composite promoter comprising the human Elongation Factor-1α (EF-1α) core promoter and the R segment and part of the U5 sequence (R-U5′) of the Human T-Cell Leukemia Virus (HTLV) Type 1 Long Terminal Repeat. The EF-1α promoter exhibits a strong activity and yields long lasting expression of a transgene in vivo. The promoter drives the expression of the anti-CD19/CD20 CAR proteins.

Kozak: Kozak consensus sequence The Kozak consensus sequence is placed in front of the start codon of the opening reading frame (ORF) of interest to facilitate translation initiation in eukaryotes.

CD19/CD20 ORF: The open reading frame of the tandem CAR T proteins.

WPRE: Woodchuck hepatitis virus posttranscriptional regulatory element (WPRE). The WPRE enhances viral RNA stability in packaging cells, leading to higher titer of packaged lentiviral particles. Here, a mutation was introduced into the translation initiation (ATG) site of the X protein to eliminate any potential translation through the WPRE promoter sequence.

3′ LTR-ΔU3: A truncated version of the HIV-1 3′ long terminal repeat that deletes the U3 region. This leads to the self-inactivation of the promoter activity of the 5′ LTR upon viral vector integration into the host genome (since the 3′ LTR is copied onto 5′ LTR during viral integration). The polyadenylation signal contained in 3′ LTR-ΔU3 serves to terminates all upstream transcripts produced both during viral packaging and after viral integration into the host genome.

pUC ori: pUC origin of replication. Plasmids carrying this origin exist in high copy numbers in E. coli.

Extracellular Domains: The extracellular domain of the pALD CD19/CD20 CAR is derived from the antigen binding domain of Leu16 murine antibody that recognizes human CD20, and the antigen binding domain of FMC63 (recognizing CD19) to create a tandem-CAR configuration. The bispecific configuration enables bivalent binding to CD19 and/or CD20 on B cells. The scFv fragments are connected via a glycine-serine (GS) flexible linker (e.g., (G₄S)₄). The IgG4 extracellular hinge connects the Leu16 scFv to the CD28 TM domain, and the intracellular domains derived from the human cytoplasmic domain of 4-1BB costimulatory domain and CD3 zeta activation domain.

scFv domains: The bispecific scFv domain of a CAR provides the targeting function by specifically recognizing the tumor antigens. The specificity of the scFv is a crucial determinant for the CAR T cell safety profile. Given that both CD19 and CD20 expression is restricted within the B-cell lineage, the tandem CARs scFv are considered reasonably safe with respect to off target cross reactivity.

Spacer and transmembrane domain. In the anti-CD19/CD20 CAR, the non-signaling Extracellular Spacer Domain is derived from the IgG4 Hinge region, while the transmembrane domain is derived from human CD28. The spacer provides a flexible link between the scFv and the transmembrane domains. It allows the antigen-binding domain to accommodate different orientations to facilitate antigen recognition. The transmembrane domain provides a physical link between the spacer and intracellular signaling domains. The length and topology of the spacer and transmembrane domains are critical in providing an appropriate steric orientation for specific antigen recognition and subsequent T cell activation.

Intracellular signaling domains. The CAR intracellular signaling domains play crucial roles in T cell activation, persistence, and effector functions. Although the CD3ζ chain is adequate for T cell activation, one or more costimulatory domains are also needed to fully activate T cells and to promote CAR T cell persistence (Milone et al., Mol. Ther. 17(8):1453-64 (2009)). These domains are typically derived from the intracellular domains of costimulatory proteins such as 4-1BB (CD137). Differences in costimulatory domain function may impact product safety and activity by affecting CAR T cell cytokine production, expansion, cytotoxicity and persistence after administration. The intracellular signaling domains of the anti-CD19/CD20 CAR are derived from human 4-1BB and CD3ζ.

Plasmid DNA was prepared from a vial of pALD_CD19/CD20 CAR MCB according to written procedures at Aldevron. Details of the plasmid production process will be provided in the IND with a letter of cross-reference to the Aldevron Drug Master File (DMF).

### Example 10: Lentiviral Vector Manufacturing

Production of the lentiviral vector is as follows. Using the current serum-free suspension manufacturing process, viral vector is manufactured by transient transfection using Lentigen's four plasmid system, which includes research grade transfer plasmid and Lentigen's three helper plasmids. The downstream process includes clarification, Benzonase® treatment, tangential flow filtration, and column chromatography. After purification, the Vector will be concentrated approximately 100-fold, formulated in formulation buffer, sterile filtered, and filled into 1 mL vials.

In preparation for GMP production of the lentiviral batch, one 2 ml Research-grade batch and 4L non-GMP batch was prepared by Lentigen.

### Production and Release of IMPT-314 Drug Product

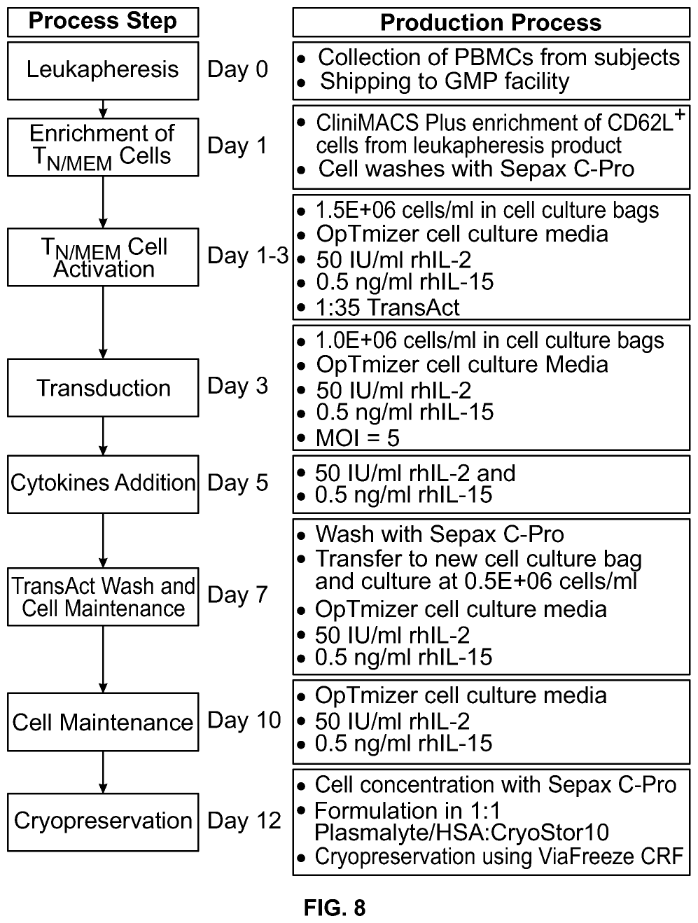

IMPT-314 Manufacturing Procedure. The proposed manufacturing process for production of IMPT-314 is provided in FIG. 8. The manufacturing process consists of the following steps and timing:

Day 0: Starting Material (Leukapheresis). Autologous peripheral blood mononuclear cells (PBMCs) is collected by leukapheresis according to written procedures. The leukapheresis product is collected into sterile bags at the collection site and shipped at 2-8° C. in a temperature-controlled container for processing and cell product manufacture.

Day 1: Enrichment and Activation of T_{N/MEM} cells. The leukapheresis product is then enriched for CD62L+ cells using GMP-grade CD62L microbeads and a CliniMACS Plus (Miltenyi Biotec). Briefly, the leukapheresis product is washed with CliniMACS PBS/EDTA buffer supplemented with 0.5% HSA. The cells are then incubated with the anti-CD62L microbeads and washed with the PBS/EDTA/HSA buffer using a Sepax C-Pro (Cytiva). CD62L microbead labeled cells are then added and run through the CliniMACS plus. The resultant selected CD62L+ cells are called naïve/memory T cells (T_{N/MEM}). T_{N/MEM} cells will be resuspended in OpTmizer™ media supplemented with 50 IU/mL human IL-2 and 0.5 ng/mL human IL-15 (Miltenyi Biotec) and transferred to static cell culture bag (VueLife). The cells are then stimulated with GMP-grade TransAct Human T cell expander CD3/CD28 (Miltenyi Biotec) added at a ratio of 1:35 and cultured for 42 hours at 37° C./5% CO₂ in a humidified incubator.

Day 3: Lentiviral Transduction. Genetically modified T cells expressing an anti-CD19/CD20 CAR are manufactured by transduction of stimulated T_{N/MEM} cells with lentiviral vector (Lentigen). To perform this unit operation, activated T_{N/MEM} cells are harvested from the static cell culture bag and re-cultured in a new cell culture bag in fully supplemented OpTmizer™ media at a density of 1.0E+06 cells/mL. Lentiviral vector is then be added to the cells at a multiplicity of infection (MOI) of 5. The cells with the added lentiviral vector are then incubated for 6 hours at 37° C./5% CO₂ in a humidified incubator. Following this incubation, an equal volume of media is added to the cell culture bag to have a final cell concentration of 0.5E+06 cells/mL and incubated at 37° C./5% CO₂ for a further 2 days.

Day 5: Cytokines addition. On day 5 post-stimulation, 50 IU/mL human IL-2 and 0.5 ng/mL human IL-15 (Miltenyi Biotec) is added to the culture bag containing the T cells. Cells are incubated at 37° C./5% CO₂ for a further 2 days.

Day 7: TransAct Wash and Cell Maintenance. After 7 days of culture, transduced T_{N/MEM} cells are washed out of the media containing TransAct using a Sepax C-Pro (Cytiva) and re-cultured in a new cell culture bag with fully supplemented fresh media at a density of 0.5E+06 cells/mL. Cells will be incubated at 37° C./5% CO₂ for a further 3 days.

Day 10: Cell Maintenance. On day 10 post-stimulation, fully supplemented media is added to dilute the cells at a cell density of 0.5E+06 cells/mL. Cells are incubated at 37° C./5% CO₂ for a further 2 days.

Day 12: Cryopreservation. After 12 total days of culture, transduced T_{N/MEM} cells are harvested and cryopreserved at a fixed dose of CAR+ cells. Cells are washed out of media and into Plasmalyte plus HSA using a Sepax C-Pro (Cytiva). The concentrated cells are then mixed at a 1:1 ratio with a cryopreservation solution containing DMSO (CryoStor CS10 Freezing Solution, BioLife). Cells are then frozen using a ViaFreeze™ controlled rate freezer (Cytiva), cryopreserved in a labeled cryopreservation-bag and stored in a centrally-monitored vapor phase liquid nitrogen freezer. The Drug Product is cryopreserved in CryoMACS Freezing Bag 50 or 250. A portion of the cells are then cryopreserved in small aliquots along with the bulk final product for final lot-release testing.

Table 4 describes the product contact materials that will be used for manufacture of IMPT-314 (pALD-Leu16-FMC63-BBz).

TABLE 4
Reagents/Materials Used in Manufacture

| Reagent/Material | Vendor-Supplier | Use |
|---|---|---|
| Human serum albumin (HSA) | Nova Biologics | Cell enrichment |
| Plasmalyte | Baxter | Final product formulation |
| CliniMACS CD62L GMP | Miltenyi | Cell enrichment |
| MicroBeads | | |
| MACS GMP T Cell TransAct | Miltenyi | Cell activation |
| CliniMACS PBS/EDTA Buffer | Miltenyi | Cell Enrichment/Depletion |
| research | | |
| GMP Recombinant Human IL-2 | Miltenyi | Cell culture media |
| GMP Recombinant Human IL-15 | Miltenyi | Cell culture media |
| GlutaMAX-I Supplement | Gibco | Cell culture media |
| CTS^{TM} OpTmizer T-Cell | Gibco | Cell culture media |
| Expansion Supplement | | |
| CTS^{TM} OpTmizer T-Cell | Gibco | Cell culture media |
| Expansion Basal Medium | | |
| Cryostor CS10 Cryopreservation | BioLife | Final product formulation |
| media | | |
| Cell culture bag | Saint-Gobain 3 197-C | Culture bag |
| Cell culture bag | Saint-Gobain 3 290-C | Culture bag |
| CryoMACS Freezing Bag 50 | Miltenyi | Final product |
| CryoMACS Freezing Bag 250 | Miltenyi | Final product |

Abbreviations:
GMP = Good Manufacturing Practice;
HSA = human serum albumin

### In Process Testing Strategy

In-Process Testing Samples are collected before, during, and at the end of the cell-manufacturing procedure. The planned list of control points is provided in the table below (Table 5).

TABLE 5

| Unit Operation | Process Step | Testing Purpose |
|---|---|---|
| Day 1: Apheresis Collection | Apheresis before Sepax wash and | Total cell count and viability |
| | volume reduction | Leukocyte subsets composition and T-cell |
| | | phenotype |
| | | Sample taken for sterility if needed |
| Day 1: CD62L Enrichment | Enriched CD62L cells before Sepax | Total cell count and viability |
| | wash | |
| | Enriched CD62L cells after Sepax wash | CD62L purity check |
| Day 7: Cell Maintenance | Before media addition | Total cell count and viability |
| Day 10: Cell Maintenance | Before Sepax wash | Total cell count and viability |

Each Drug product will be tested for release criteria (See Table 6).

TABLE 6
IMPT-314 Release Criteria

| Attributes | Product metric | Method | Limits |
|---|---|---|---|
| Purity | Appearance | Visual inspection | Off-white, |
| | | | product free of |
| | | | particulates |
| Dose | Number of CAR T cells | % CAR+ by Flow | DL1: 50E6 +/− |
| | manufactured | Cytometry | 10E6 CAR+ cells |
| | | CAR+ Cell count by | DL2: 150E6 +/− |
| | | NucleoCounter | 30E6 CAR+ cells |
| Potency | Viability | NucleoCounter | ≥70% |
| | CAR+ expression | CAR+ by F(ab)₂ | ≥10% |
| | | detection Flow | |
| | | Cytometry | |
| | Tumor reactivity | IFN-y secretion by | CD19+ target |
| | | | cells: ≥100 pg/ml |
| | | ELISA | CD20+ target |
| | | | cells: ≥100 pg/ml |
| | CTL | Flow Cytometry | TBD |
| Purity | CD3% | Flow Cytometry | ≥80% |
| | Endotoxin | EndoSafe-PTS | ≤5 EU/ml |
| Identity | Target vector inserted | ddPCR using Leu16 | Amplified above |
| | gene | specific primers/probe | LOD |
| Safety | VCN | ddPCR | ≤5 copies/cell |
| | RCL | qPCR | Not detected |
| Attributes | Product metric | Method | Limits |
| Sterility | Bacterial and Fungal | USP <71> or Bac-T Alert | No Growth |
| | culture | | |
| | Gram stain | Staining | Negative |
| Mycoplasma | Mycoplasma | MycoAlert | Not detected |

DL: Dose level;
CAR: chimeric antigen receptor;
CTL: cytotoxic T lymphocytes;
ddPCR: droplet digital polymerase chain reaction;
ELISA: enzyme-linked immunosorbent assay;
IL-2: interleukin-2;
IFN-γ: interferon gamma;
qPCR: quantitative polymerase chain reaction;
RCL: replication-competent lentivirus;
VCN: vector copy number

### Analytical Methods

Viability and Cell Count: The Number of cells and viability is assessed using an automated cell counter system (Nucleocounter NC-202). Viability in the Final Drug Product (collected at day 12) is measured.

CAR Expression: Percentage of CAR+ cells is assessed by Flow Cytometry using a polyclonal antibody against mouse F(ab)2. Live/Dead stain is included in the panel to discriminate the positive cell population. Isotype control is used to identify the non-specific binding. Flow Cytometry assays are performed on a representative aliquot of the Drug Product collected and cryopreserved at day 12.

Vector Copy Number: Vector copy number (VCN) estimates the number of vector copies in each transduced cell, which may correlate with the amount of CAR protein expressed on the cell surface. VCN is determined by ddPCR, using primers/probes sets specific to amplify the CD20 scFv derived from the Leu-16 mAb. The average number of integrations per CAR-positive cell is determined.

Cytokine secretion (IFN-γ secretion): To assess potency of the IMPT-314, a representative aliquot of the cryopreserved Final Product is thawed to perform cytokine assays. Drug Product and target cells are co-cultured at varying ratios of effector to target (E:T) in the absence of exogenous cytokines and incubated for 16-18 hours. Raji target cells are engineered to express 1) CD19 antigen only, and 2) CD20 antigen only to assess the activity of both elements in the CAR. CD19 and CD20 knockout (KO) target cell lines are generated from parental wildtype Raji cells (CD19+ and CD20+) using CRISPR/Cas9 and expanded from single cell clones. CEM cells, which do not express CD19 or CD20, were used as a negative control. After incubation, supernatants are harvested, diluted 10×, and analyzed for secretion of IFN-γ using R&D Systems Quantikine ELISA kits.

Cytotoxic T Lymphocytes assay (CTL): To show specific killing against CD19 and CD20 expressing Raji cells, a co-culture of GFP-expressing Raji cells (wildtype, CD19KO and CD20KO) or CEM (CD19−/CD20−) with transduced T cells at E:T titrations is used. Raji cell viability is analyzed by Flow Cytometry after 18 hours and gated on Sytox Blue negative and GFP positive target cell counts.

Sterility: A representative aliquot of the Final Drug Product is tested for bacterial and fungal growth by USP <71> or Bac-T Alert. A sample of the leukapheresis starting material is retained for post hoc testing in the event of a Final Drug Product sterility test failure.

Endotoxin: Endotoxin levels in the Final Drug Product is assessed using the Charles River EndoSafe™ Portable Test system.

Mycoplasma: Mycoplasma is tested using the MycoAlert Mycoplasma Real-Time PCR Kit. The MycoAlert™ system is a biochemical reaction test that detects the presence of mycoplasmal enzymes that are not found in eukaryotic cells.

RCL: The Drug Product is assayed to detect RCL using qPCR for VSV-G sequences. qPCR assay can be called negative if the final value is less than the LOQ. Optional RCL-cell culture assay with amplifying/indicator cell lines is submitted and performed only if the qPCR criteria is not met, for confirmation.

Direct Detection of CAR+ cells by Flow Cytometry. Direct measurement was used to detect expression of anti-CD19/CD20 CAR. Because quantification of a tEGFR protein may not provide an accurate measurement of surface CAR expression, tEGFR was removed from the Lentiviral vector as described herein, and instead utilized antibodies which bound to the scFv domains of the anti-CD19/CD20 CAR protein to report surface CAR expression. To identify the best detection reagent, a panel of antibodies, proteins, and peptides was screened, which found that one antibody in particular—anti-mouse F(ab)2 antibody (Jackson Immuno Research) showed superior CAR detection in multiple donors.

TABLE 7
Comparison of antibodies, proteins,
peptides to detect anti-CD19/CD20 CAR

| Antibody/Protein | Vendor | % Positive Cells |
|---|---|---|
| CD19 Fc HisTag | Sino Biotech | 16.2 |
| CD19 Fc | R&D Systems | 39.2 |
| CD20 Protein-Biotin | Acros | 68.1 |
| CD20 Peptide-FAM | GenScript | 8.3 |
| Byotinilated anti- | Acros | 59.6 |
| FMC63 | | |
| Anti-mouse F(ab)2 | Jackson Immuno | 84.9 |
| | Research | |
| Goat anti-Ms IgG | Abcam | 77.6 |
| (H + L) -AF488 | | |
| Goat anti-Ms IgG | Abcam | 77.8 |
| (H + L) -AF 647 | | |
| Goat anti-Ms IgG | Invitrogen | 78.0 |
| (H + L) -AF488 | | |

T cells from donor 1917 transduced with CD 19/20 CAR lentiviral vector were washed and stained using various antibodies, proteins, or peptides. Antibodies were titrated from 1:25 to 1:200. Anti-F(ab)2 biotin antibody (Jackson Immuno Research) and anti-ms IgG (H&L) 647 (Abcam) stained cells with similar efficacy compared to EGFR stain.

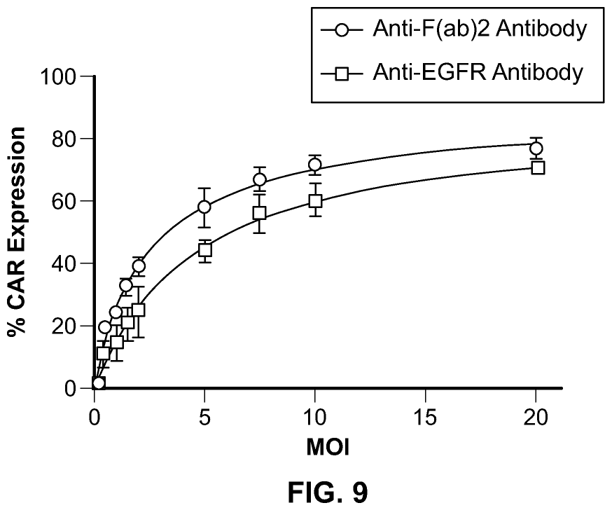

Next, to compare the direct CAR detection method to a tEGFR detection method, a lentiviral vector containing the anti-CD19/CD20 CAR and tEGFR transgenes was prepared, and had this material prepared at Lentigen, (i.e., a comparator vector). Leukapheresis material from 3 different donors was processed following CD62L+ enrichment and activation steps described herein. T cells were transduced at day 3 with CC310B and expanded in culture until day 12, following manufacturing protocol herein. T cells were collected and stained with either anti-mouse F(ab)2 (Jackson ImmunoResearch) or anti-human EGFR antibodies against tEGFR. As shown in FIG. 9, cells stained with anti-mouse F(ab)2 or anti-human EGFR behaved similarly at a range of MOIs, suggesting that anti-F(ab)2 antibody can be used to accurately and directly measure CAR expression level on the cell surface. CAR detection with the anti-mouse F(ab)2 antibody is thus useful to measure CAR transduction—all process runs in this section were performed using anti-F(ab)2 antibody to measure CAR expression.

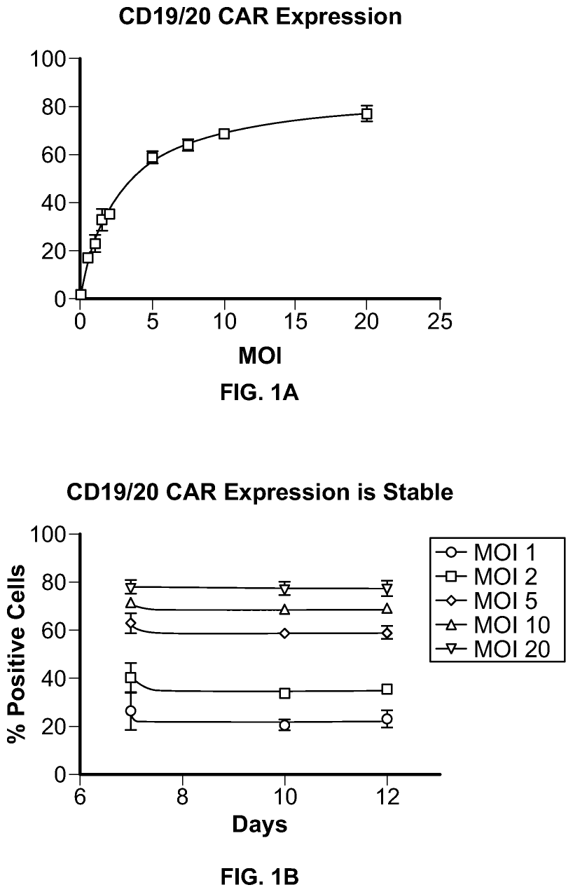

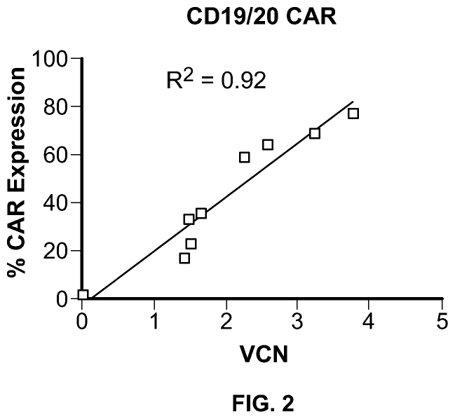

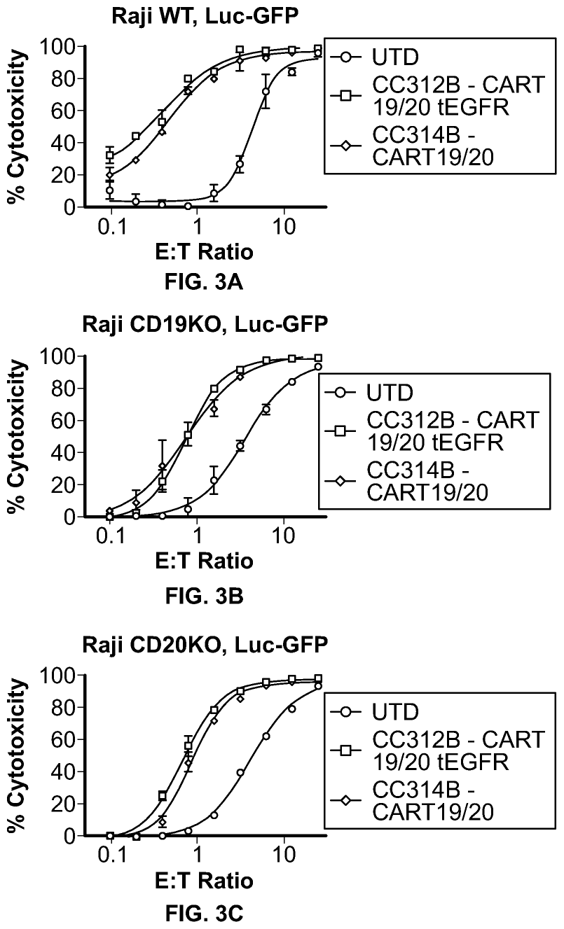

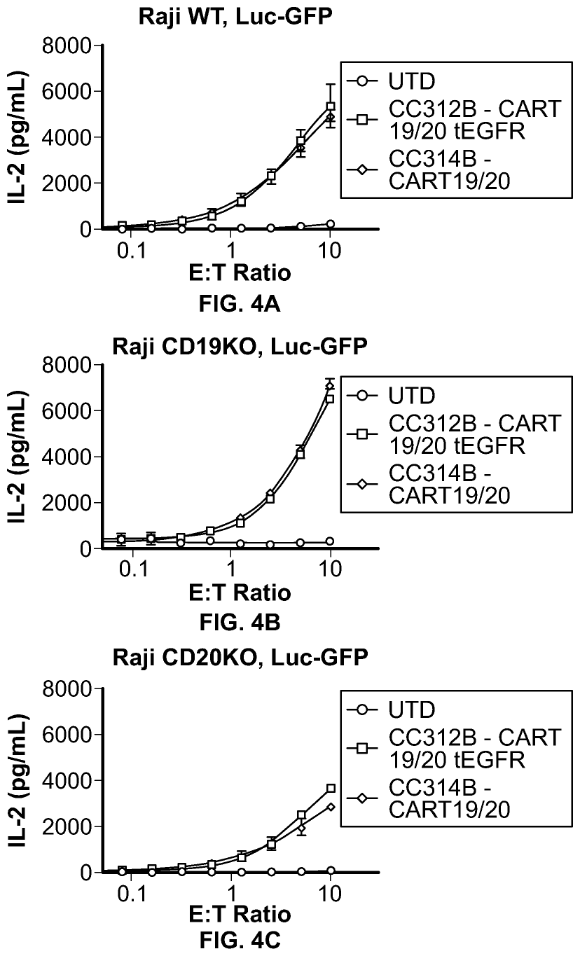

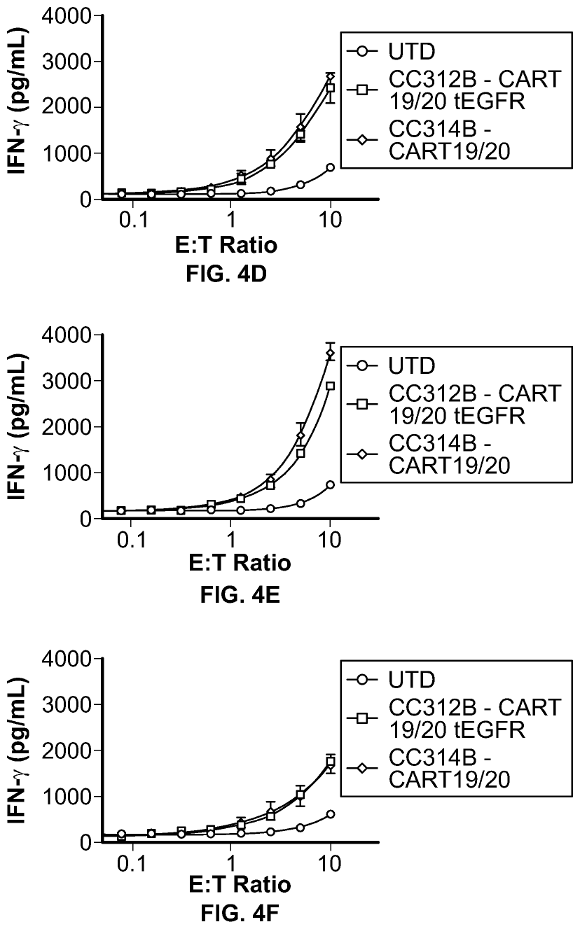

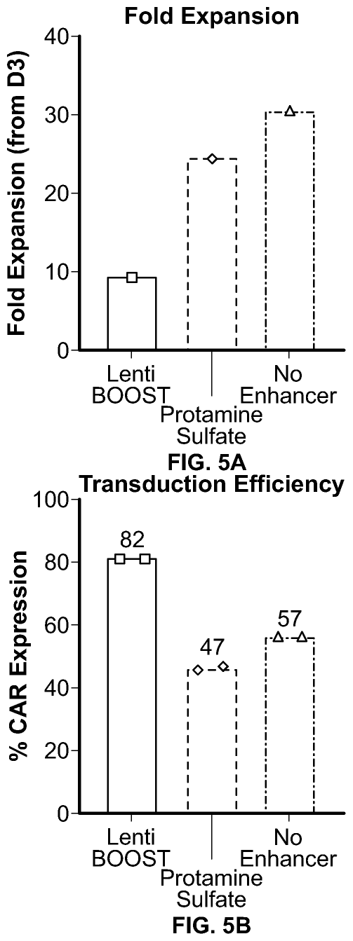

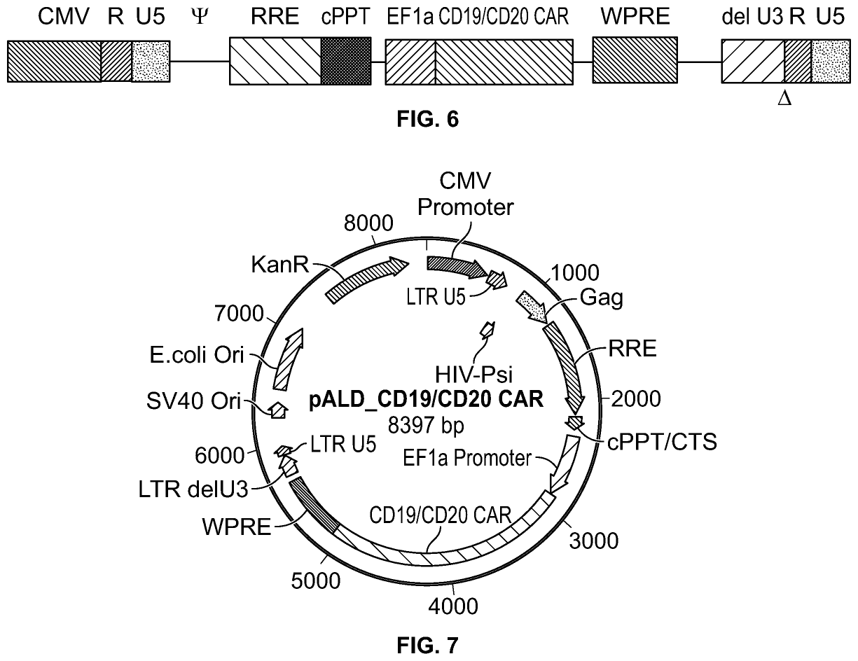

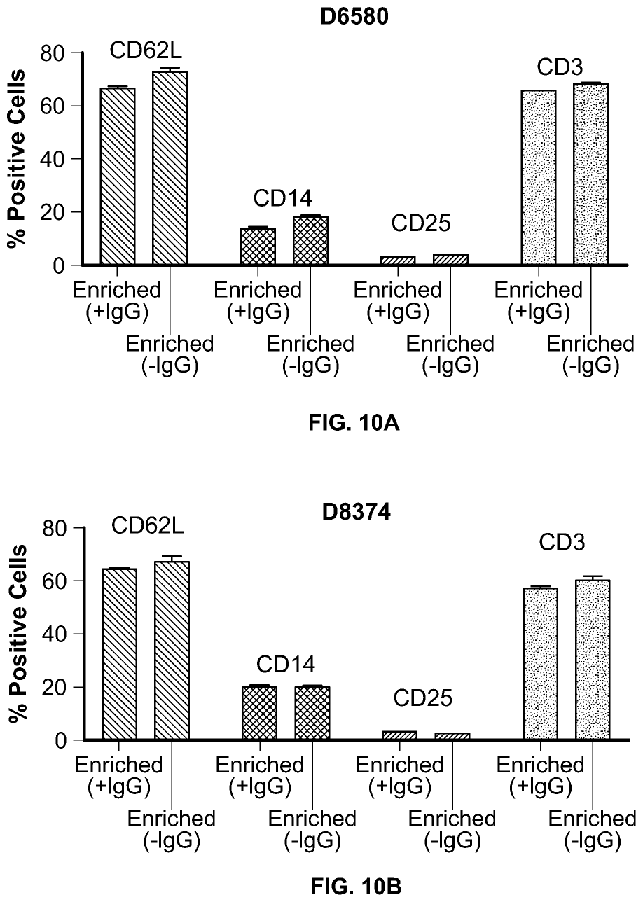

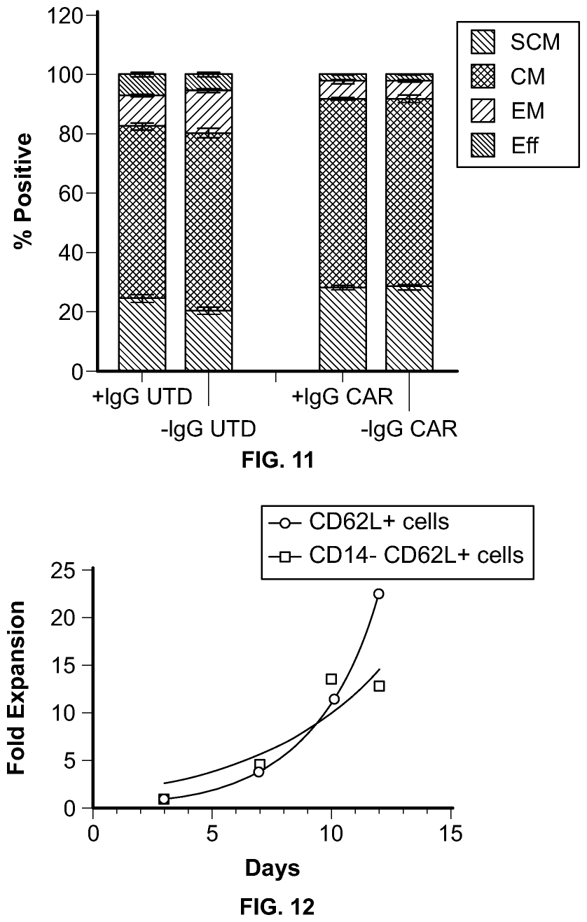

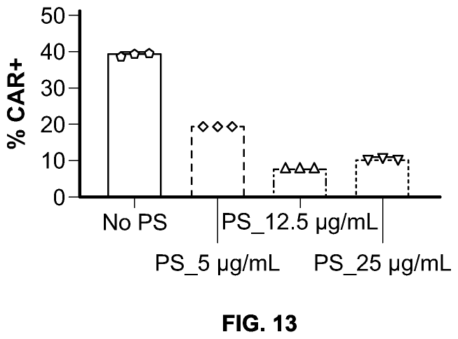

CD62L+ enrichment step: In two separate PD runs, it was investigated whether inclusion of IgG as a blocking reagent in the depletion step was helpful during the CliniMACS™ CD62L-selection step. Prior processes employed human IgG (Gammagard, Takeda) during CD62L enrichment to potentially increase the purity of the enriched cells. The intention was to decrease unwanted antibody binding (of the murine anti-CD62L monoclonal antibodies) to Fc receptors present on immune cells (such as B lymphocytes, dendritic cells, monocytes, macrophages, NKs, etc.). Side-by-side comparison of the enrichment process performed with and without IgG (Gammagard and Human IgG Affinity Purified Low Endotoxin, Innovative Research) in two different healthy donors (6580 and 8374, respectively), showed identical CD62L+ enrichment (FIGS. 10A-B). Additional markers were analyzed to determine the impact of the IgG on various cell populations. The percentage of CD3+, CD14+ and CD25+ cells is essentially the same after enrichment regardless of whether IgG is included (FIG. 1A and FIG. 1B, respectively). Cells selected with and without IgG were activated, transduced with CD 19/20 CAR lentiviral vector and expanded following the manufacturing process to explore potential impact on T cell Immunophenotype. As shown in FIG. 11, no differences were observed with or without IgG during CD62L+ selection. Results showed that IgG does not have impact on the purity of the enriched cells and therefore will not be used as a reagent in the manufacture process.

CD14+/CD25+ depletion step: As noted herein, removal of CD14/CD25− positive cells was an optional step, and in 3 out of 8 clinical material manufactured, this step was not included though clinical outcomes were favorable. It was therefore evaluated if the step to deplete CD14+ and CD25+ cells after CD62L+ enrichment was critical for successful transduction and expansion of CAR T cells. Others have shown that a high monocytes content can inhibit the activation and expansion of T cells, likely because of the immunosuppressive functions of the CD14+ cell, in addition to the unspecific sequestration of the CD3/CD28 activation beads by the monocytes (Stoncek, 2016; Wamg 2021). To explore this potential immunosuppressive effect, a side-by-side manufacturing process was performed comparing the expansion and transduction efficiency of the T cells with or without the depletion step. Similar level of T cells fold expansion, following TransAct activation, was observed in CD62L+ enriched cells and CD62L+ enriched/CD14+ depleted cells at different timepoints after transduction (FIG. 12). Moreover, transduction efficiency has been assessed between the two processes. No difference in CAR expression level was observed between the cells processed with and without the depletion step (30.5% and 27.3%, respectively). The percentage of monocytes during T cell culture was also assessed in PD runs using T cells from 3 different donors. Results showed that after the initial CD62L+ enrichment step, the percentage of CD14+ cells in the total population at day 3 (day of transduction) had dropped to ≤3%, Table 9), and did not interfere with activation and transduction of the T cells.

TABLE 8
Percentage of CD14+ cells at day 1 and day 3 in 3 different donors.

| Product Metric | Method | | D0597 | D6580 | D1114 |
|---|---|---|---|---|---|
| Fold Expansion | Calculated based on cell number | | 22 | 19 | 31 |
| | seeded at Day 3 | | | | |
| Viability | NucleoCounter | | 88% | 93% | 98% |
| CAR⁺ | CAR⁺ by F(ab)2 detection Flow | | 31% | 40% | 57% |
| expression | Cytometry | | | | |
| Tumor | IFN-γ | CD19⁺ Raji | 2456 pg/mL | 339 pg/mL | 2852 pg/mL |
| reactivity | secretion by | | | | |
| | ELISA | CD20⁺ Raji | 1872 pg/mL | 431 pg/mL | 4154 pg/mL |
| | (10:1) | | | | |
| VCN | ddPCR | | 2.0 | 1.9 | 2.1 |

| | | | | |
|---|---|---|---|---|
| CD3% of Live | Flow Cytometry | 97% | 94% | 98% |
| CD4% of CD3 | Flow Cytometry | 45 | 40 | 61 |
| CD8 of CD3 | Flow Cytometry | 53 | 54 | 37 |
| CD45RA⁺ | Flow Cytometry | 33 | 43 | 33 |
| CD62L⁺ | | | | |
| (Naive) | | | | |
| CD45RA⁻ | Flow Cytometry | 61 | 51 | 63 |
| CD62L⁺ (CM) | | | | |
| CD45RA⁻ | Flow Cytometry | 5 | 4 | 3 |
| CD62L⁻ (EM) | | | | |

In total, these data support that the CD 14 depletion step is not required to allow T cell transduction and expansion, and the CD14+/CD25+ depletion step for clinical will not be performed for manufacturing.

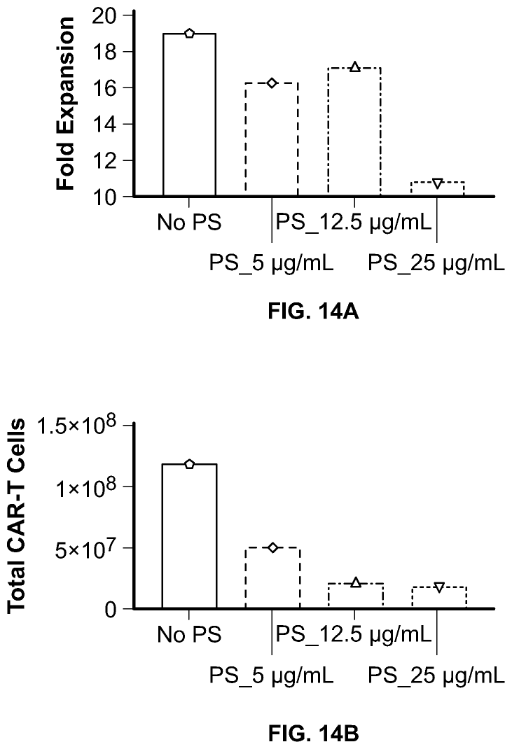

Transduction. It was then tested whether the addition of protamine sulfate, used as a transduction enhancer is necessary to achieve a consistent T cell transduction. T cells from donor 6580 (D6580) were enriched and activated as described herein. At day 3, T cells were transduced with or without protamine sulfate. Cells were expanded and collected for testing at day 12. Flow Cytometry results showed that the percentage of CAR+ cells is higher without protamine sulfate than in the groups transduced with protamine sulfate. In addition, protamine sulfate had a detrimental effect on cell expansion and therefore on the final yield of CAR+ cells (collected at day 12) (FIG. 14A and FIG. 141B, respectively).

Process Development Runs. To test the manufacturing changes adopted by the sponsor, leukapheresis material of 3 healthy donors was used to manufacture 3 lots of IMPT-314B. The parameters assessed by the studies were: (a) Cell expansion; (b) transduction efficiency measured by flow cytometry and vector copy number; (c) Potency of the anti-CD19/CD20 CAR T cells; and d) immunophenotyping. Results from these studies, compiled from 3 healthy donors, are described in the sections below.

Fold-expansion and viability of 3 different IMPT-314 lots. Transduced T cells were sampled on days 7, 10, and 12 of the manufacturing process for assessment of CD3 and CAR expression using flow cytometry. Cell count and viability was assessed using a Nucleocounter™ on days 1, 3, 7, 10, and 12 of the process. During the manufacturing process days 3 to 12, transduced T cells expanded on average 5- to 12-fold Error! Reference source not found.). Cell viability remained greater than 80% throughout the entire process.

IMPT-314 showed stable and high transduction efficiency. Transduced T cells were collected to assess anti-CD19/CD20 CAR expression. On days 7, 10, and 12 of the PD runs, CAR expression was measured using anti-F(ab)2 antibody. High level of CAR expression was detected (55-70%) and remained stable from day 7 to day 12 of the process.

Vector copy number. Transduction efficiency was further assessed by measurement of VCN per transduced cell. T cells were harvested on day 12 at the end of the manufacturing process and total number of copies of vector integrated into the host genome was quantified by droplet digital PCR and normalized to percentage of CAR positive cells reported by flow cytometry. VCN ranged from 2 to 3 per transduced cell in all 3 PD runs.

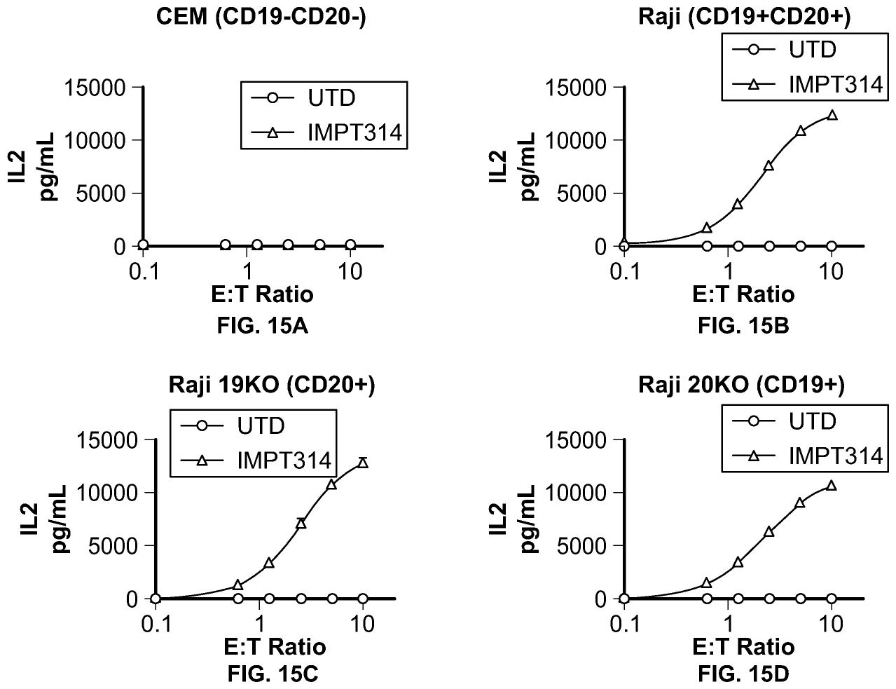

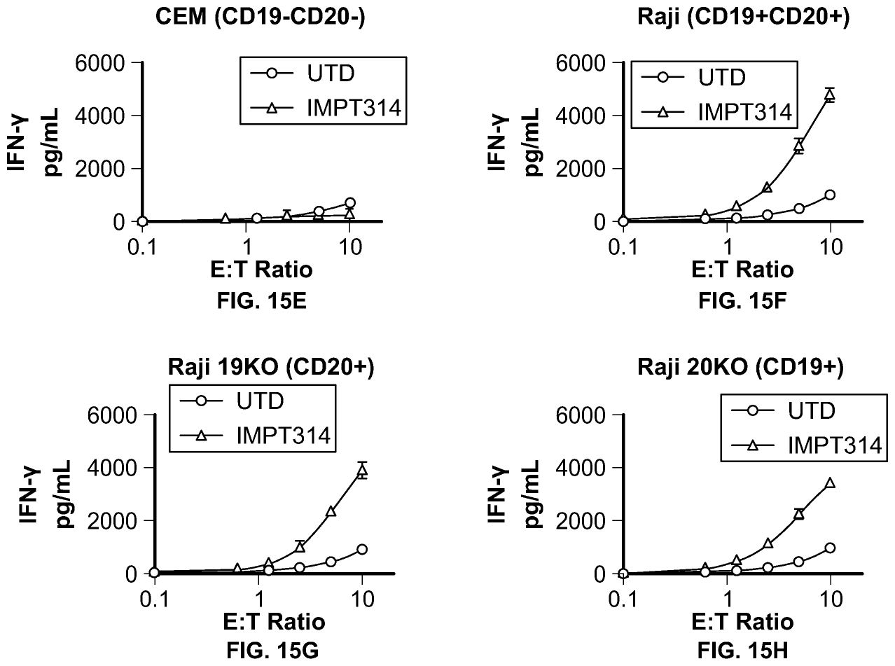

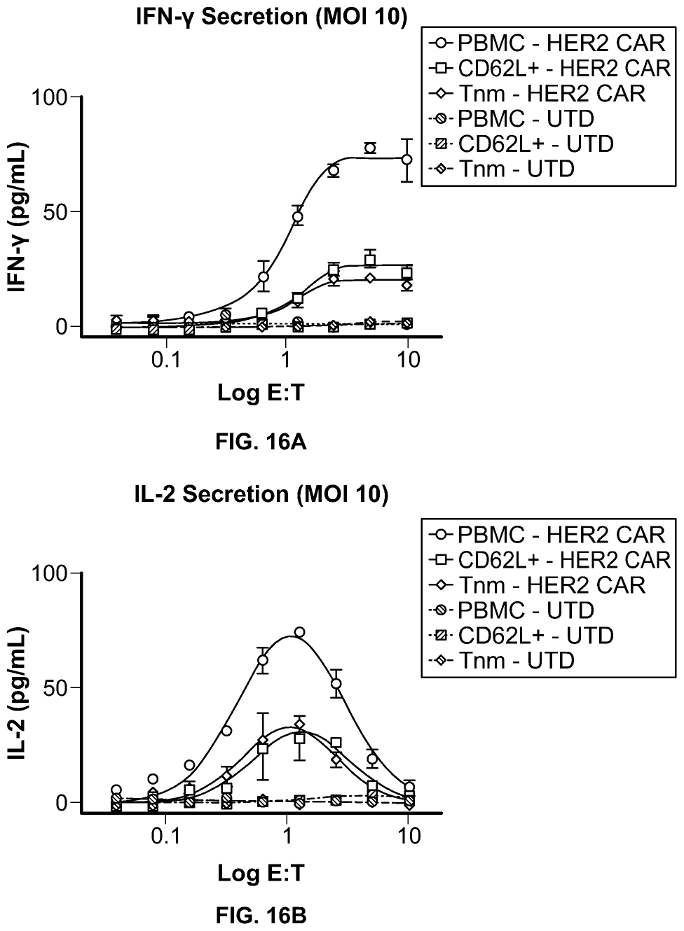

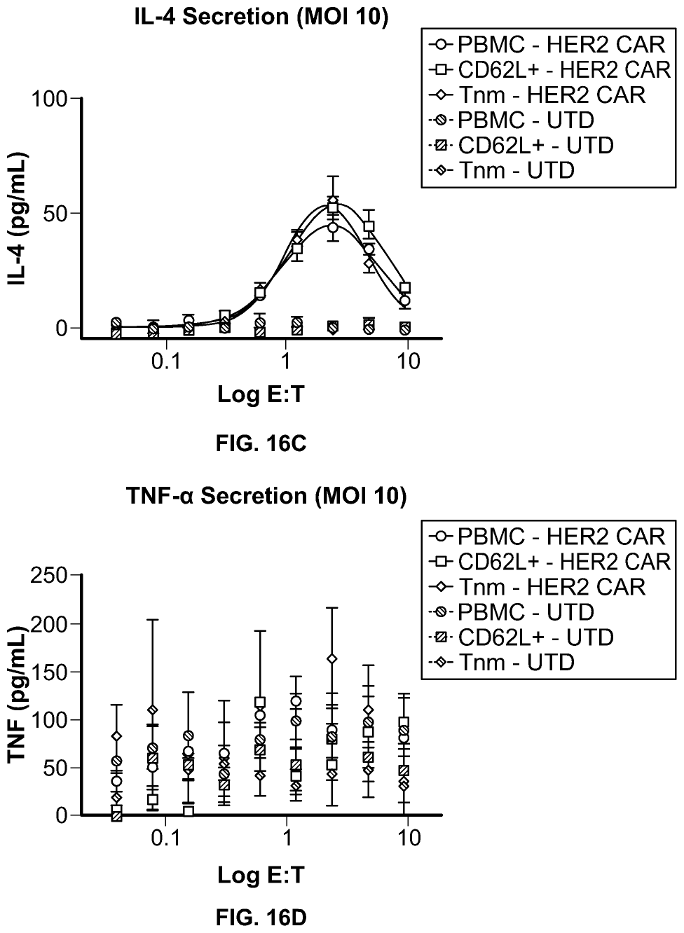

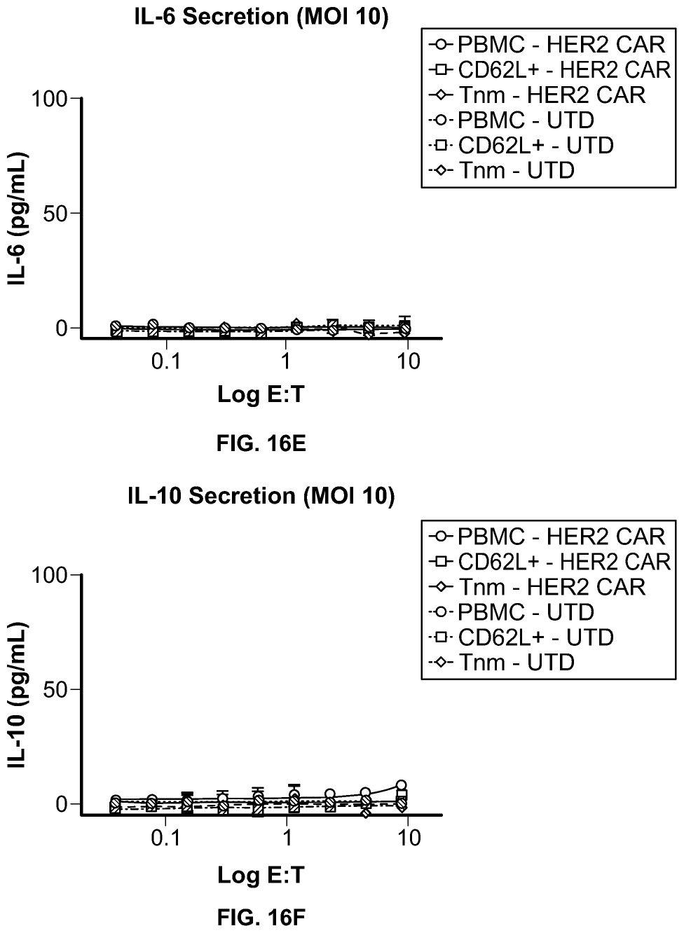

Potency of the anti-CD19/CD20 CAR T cells as a drug product. After characterization, potency of the anti-CD19/CD20 CAR T cells was evaluated using cryopreserved CAR T cells at the end of the process run to evaluate the ability of CAR T cells to secrete inflammatory cytokines when co-cultured with antigen positive target cells. To examine efficacy of each scFv domain of the bispecific anti-CD19/CD20 CAR, Raji cells were engineered to express 1) CD19 antigen only, and 2) CD20 antigen only. CEM cells, which do not express CD19 or CD20, were used as a negative control. Freshly thawed anti-CD19/CD20 CAR T cells and target cells were co-cultured at varying ratios of effector to target (E:T) in the absence of exogenous cytokines and incubated for 16-18 hours. After incubation, supernatants were analyzed for secretion of IL-2 and IFN-γ using R&D Systems ELISA kits. Error! Reference source not found. FIGS. 15A-H demonstrate CAR T cell secretion of both proinflammatory cytokines after co-culture with CD19 and/or CD20 positive Raji cells. In comparison, untransduced T cells did not secrete IFN-gamma or IL-2 when co-culture with Raji cells. IMPT-314 showed robust cytokine secretion against target cell expressing CD19/C20 and single antigen (both CD19 only and CD20 only).

Immunophenotyping. On day 12, untransduced and CD19/CD20 transduced cells were immunophenotyped with a panel of antibodies to detect CD62L, CCR7, CD45RA, CD45RO, CD3, CD4, and CD8. At the end of the manufacturing process, >80% of untransduced and CAR T cells remained stem cell memory/naïve and central memory, consistent with initial selection for CD62L positive cell population.

Flow Cytometry panel also showed a high percentage of CD3+ cells, above 96% and a similar percentage of CD8 and CD4. Potency assays.

Potency of the anti-CD19/CD20 CAR T cells was evaluated using cryopreserved CAR T cells to evaluate the ability of CAR T cells to secrete inflammatory cytokines when co-cultured with antigen positive target cells. Briefly, freshly thawed IMPT-314 and target cells were co-cultured at varying ratios of E:T in the absence of exogenous cytokines and incubated for 16 to 18 hours. After incubation, supernatants were analyzed for secretion of IFN-γ using R&D Systems ELISA kit. FIGS. 18A-L show increased secretion of IFN-γ by CAR T cells after co-culture with CD19 and/or CD20 positive Raji cells at increasing E:T ratios, demonstrating specific activity of both the anti-CD19 and anti-CD20 element of IMPT-314.

The data from runs using this closed process demonstrated that the manufactured drug products met all of the release criteria set for the future GMP IMPT-314. The Drug Products showed excellent expansion and viability. The expression of the CAR was within range and stable, and the VCN was in the expected range. In each case, the IMPT-314 products had a high level of purity, with CD3+ cell content above 95%, with desirable central memory and naive content. Finally, the products specifically target tumor cells expressing CD19 and/or CD20, meeting the potency requirement.

Stability Studies. The studies to determine the stability of IMPT-314 final cell product are summarized in Table 9 for short-term and Table 10 for long-term stability studies, respectively. The long-term stability of the drug product is assessed in 3 healthy donors. Aliquots (10-20 ml) from Drug Product are collected into several (6-8) representative containers (CryoMACS Freezing bags).

TABLE 9
Planned Drug Product Short-Term Stability Studies
Drug Product Stability Study - Short Term

| | | Time Points | | |
|---|---|---|---|---|
| | | 1 | 2 | 4 |
| Test | Method | hour | hours | hours |
| Viability | NucleoCounter | [symbol] | [symbol] | [symbol] |
| | Acridine Orange/DAPI | | | |
| Recovery | NucleoCounter | [symbol] | [symbol] | [symbol] |
| | Acridine Orange/DAPI | | | |
| % CAR+ | Flow Cytometry | [symbol] | [symbol] | [symbol] |

TABLE 10
Planned Drug Product Long-Term Stability Studies
Drug Product Stability Study - Long Term

| | | Time Points | | | |
|---|---|---|---|---|---|
| | | 1 | 2 | 6 | 12 |
| Test | Method | month | months | months | months |
| Viability | NucleoCounter | [symbol] | [symbol] | [symbol] | [symbol] |
| | Acridine Orange/DAPI | | | | |
| VCN | ddPCR | [symbol] | [symbol] | [symbol] | [symbol] |
| % CAR+ | Flow Cytometry | [symbol] | [symbol] | [symbol] | [symbol] |
| IFN-γ | ELISA | [symbol] | [symbol] | [symbol] | [symbol] |
| secretion | | | | | |
| CTL | Flow Cytometry | [symbol] | [symbol] | [symbol] | [symbol] |
| Sterility | Gram Stain and | | | | [symbol] |
| | Bacterial/Fungal | | | | |
| | culture | | | | |

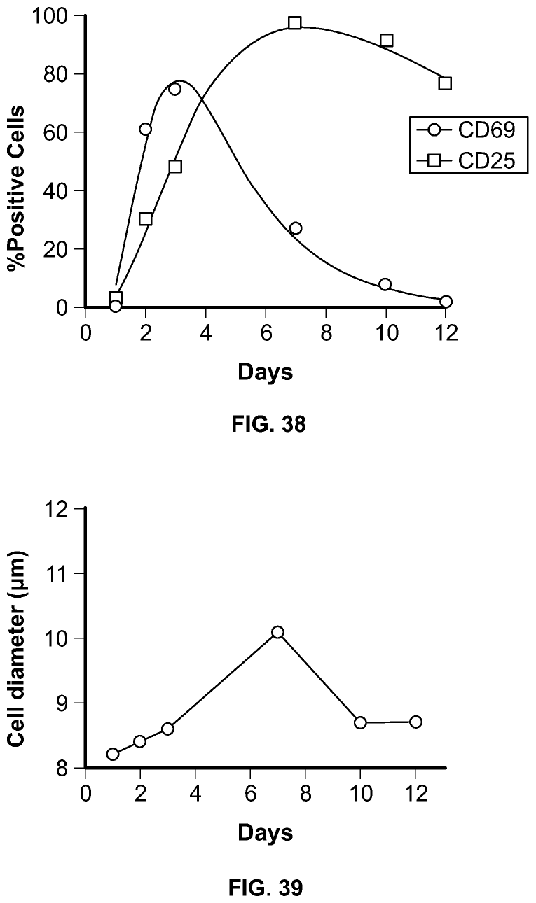

Characterization of T cell activation level during the manufacture. T cell activation level was measured by staining for T cell activation markers (CD25 and CD69) on days 1, 2, 3, 7, 10, and 12 of the manufacturing process of this example. It was observed that (1) the early activation marker CD69 peaked on day 3; and (2) CD25 expression peaked on day 7, followed by a gradual decrease after removal of TransACT (FIG. 38). Additionally, cell size was also monitored as a marker of T cell activation. Following activation with TransACT, cell diameter increased from about 8 μm on day 1 to about 10 μm on day 7, and gradually decreased after removal of TransACT on day 7 (FIG. 39). The data suggest that the manufacturing method described herein results in improved quiescence of the T cells after removal of the transactivation agent.

### Example 11. Characterization of Anti-CD19/CD20 CAR Constructions with Spacer Variations

Ten CD19/CD20 targeting CAR constructs with different spacers were constructed and evaluated. All constructs tested are summarized in Table 11:

TABLE 11
Summary of anti-CD19/CD20 CAR Constructs

| | Expression | | | | | | |
|---|---|---|---|---|---|---|---|
| | vector | CAR T | | | | | |
| Construct # | backbone | Target | | Spacer | TM | Costim | Activation |
| CC314 | pALD | CD20 | CD19 | IgG4 | CD28 | 41BB | CD3z |
| CC321 | pALD | CD20 | CD19 | CD8a-45 aa | CD28 | 41BB | CD3z |
| CC322 | pALD | CD20 | CD19 | CD28 | CD28 | 41BB | CD3z |
| CC323 | pALD | CD20 | CD19 | CD8a-45 aa | CD8 | 41BB | CD3z |
| CC324 | pALD | CD20 | CD19 | CD4 hinge | CD28 | 41BB | CD3z |
| CC325 | pALD | CD20 | CD19 | CD8a-29 aa | CD28 | 41BB | CD3z |
| CC326 | pALD | CD20 | CD19 | CD8a-18 aa | CD28 | 41BB | CD3z |
| CC327 | pALD | CD20 | CD19 | IgG1 | CD28 | 41BB | CD3z |
| CC328 | pALD | CD20 | CD19 | IgG3 | CD28 | 41BB | CD3z |
| CC329 | pALD | CD20 | CD19 | IgA2 | CD28 | 41BB | CD3z |

TM: transmembrane domain;
Costim: costimulatory domain

### Manufacturing of Anti-CD19/CD20 CAR T Cells

Anti-CD19/CD20 CAR T cells were manufactured using cells obtained from four healthy donors (D292, D4091, D010, D217). For this, on day 1, PBMCs were isolated from donor leukapheresis using Ficoll-Paque density gradient centrifugation process, followed by CD62L enrichment using anti-CD62L microbeads (Miltenyi) and positive selection of CD62L-positive cells using a LS column (Miltenyi). Post isolation, CD62L enriched cells were activated using research-grade TransAct Human T cell expander CD3/CD28 at a 1:100 dilution, supplemented with 50 IU/ml of IL-2 (Miltenyi Biotec) and 0.5 ng/mL IL-15 (Miltenyi Biotec). Two days after activation, cells were transduced with lentiviral vector produced by Vector Builder at MOI (multiplicity of infection) of 10, based on titer reported by Vector Builder. Media and cytokine additions were performed on days 5, 7, and 10. TransAct™ was removed on day 7. On day 12, cells were harvested and potency assays and flow staining was performed.

### Flow Cytometry Analysis of CAR Expression and Phenotype

About 100,000 cells (both CAR transduced and un-transduced controls) were harvested into 96 well v-bottom plates and washed 3 times with staining buffer containing 0.2% BSA. Cells were incubated in 100 μL antibodies (Table 12) diluted at 1:100 and incubated at 4° C. for 30 minutes, protected from light. After incubation, cells were washed 3 times with staining buffer and incubated with anti-mouse IgG secondary antibody (1:200) for 30 minutes at 4° C., protected from light. After incubation, cells were washed 3 times with staining buffer and resuspended in 200 μl of staining buffer and analyzed on Attune NxT flow cytometer (Thermo Fisher). Live cells were gated using forward scatter area (FSC-A) and side scatter area (SSC-A) and single cells were gated using FSC-A and forward scatter height (FSC-H).

TABLE 12
Summary of Antibodies for CAR and T cell Phenotyping
Antibody Panel

| Ab | Fluorophore | Clone | Manufacturer/Catalog # |
|---|---|---|---|
| CD3 | PerCP-Cy5.5 | SK7 | BioLegend: 344808 |
| CD4 | APCCy7 | RPA-T4 | BD: BDB557871 |
| CD8 | PE/Cy7 | SK1 | BioLegend: 344712 |
| CD45RA | FITC | HI100 | BioLegend: 304106 |
| CD62L | e450 | DREG-56 | eBioscience: 50-245-999 |
| Mouse anti-Leu16 scFv | None | Custom made | Accurus Biosciences |
| Anti-Idiotype | | | |
| Goat anti-mouse IgG (H + L)- | AF647 | Polyclonal | Invitrogen: A-21235 |
| AF647 | | | |

Potency Characterization with CTL and IFN-γ Assay

Prior to initiation of potency assays, anti-CD19/CD20 CAR T cells were thawed and rested overnight in RPMI media supplemented with 10% FBS and 1% P/S. On the day of the assay, anti-CD19/CD20 CAR T cells and un-transduced (UTD) cells were counted using Countess 3 (Invitrogen) to determine viable cell concentration. Wild-type (WT) Raji cells expressing both CD19 and CD20 antigens, CD19 knockout (KO) Raji cells, CD20 KO Raji cells, and CEM (CD19−/CD20−) cells expressing firefly luciferase were plated in a 384 well plate (2000 cells per well). T cells were added to the target cells starting at effector to target ratio (E:T) of 10:1 and serially titrated down for a total of 9 dilutions. T cells were co-cultured with target cells for 24 hours and 15 μL of supernatant was removed and analyzed using human IFN-γ cytometric bead array (CBA) kit, following manufacturer's protocol. 72 hours after the start of co-culture, Bio-Glo (Promega) was added, and luminescence (from viable target cells) was measured on the Varioskan.

### CAR Expression and Phenotyping

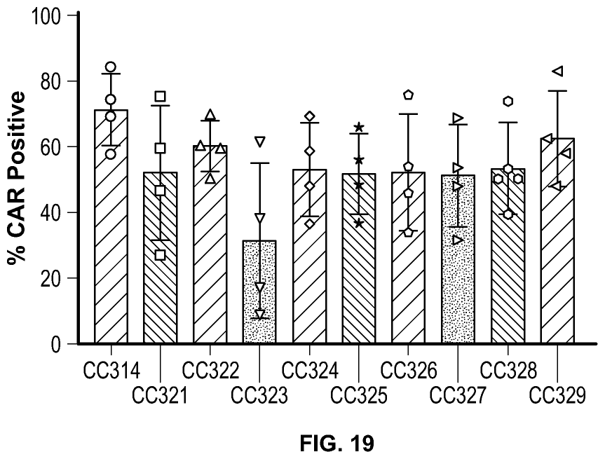

Four donors (D292, D4091, D010, D217) were transduced with constructs expressing anti-CD19/CD20 CAR (Table 11). On day 12 of manufacturing (day of harvest), surface CAR expression was measured by staining CAR T cells using antibody against Leu16. As shown in FIG. 19, CAR expression was comparable for all constructs except CC323, which showed lower % of CAR expression.

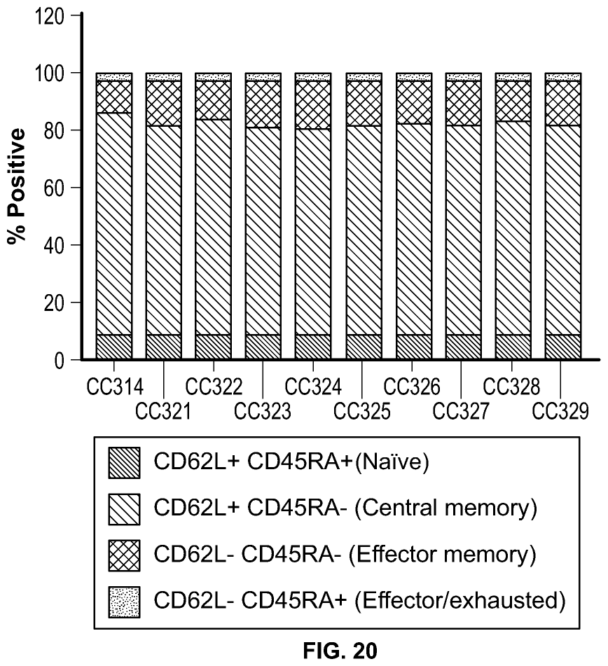

T cell Phenotype characterization was performed using a panel of antibodies (Table 12). Briefly, about 1×10⁵ cells were harvested and washed using staining buffer containing 0.2% BSA (BD Biosciences) and aliquoted into tubes and stained with the antibodies in 50 μL of staining buffer and incubated at 4° C. for 30 minutes in the dark. After staining, cells were washed 3 times with staining buffer. All flow cytometry experiments were performed on the Attune NxT flow cytometer (Thermo Fisher). As shown in FIG. 20, about 80-85% of cells remained CD62L-positive on day 12, with about 70% central cells and about 10% naïve/stem cell memory cells. CD62L-negative populations consisted of about 10-20% effector memory cells and less than 5% of exhausted phenotype.

### Potency Characterization by Cytotoxic T Lymphocyte (CTL) Assay and IFN-γ Secretion Assay

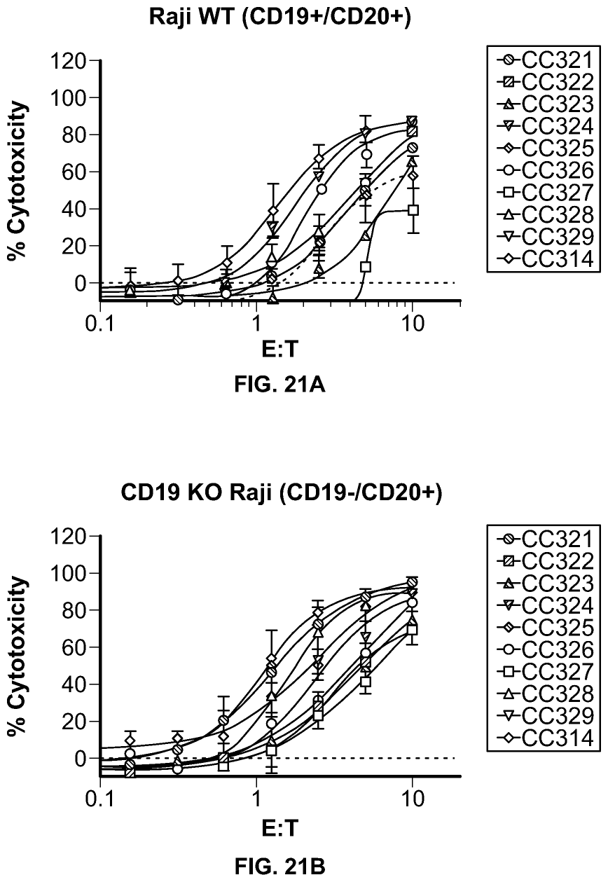

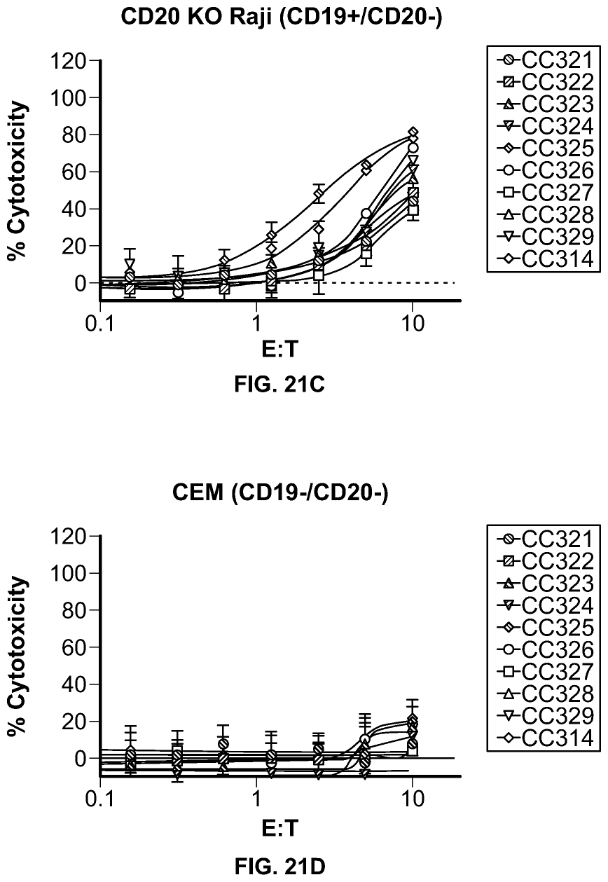

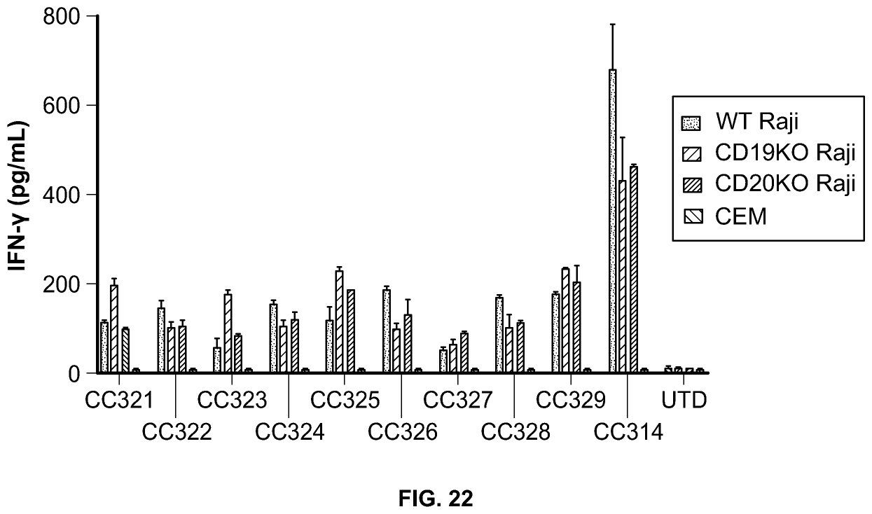

T cells transduced with anti-CD19/CD20 CAR constructs were co-cultured with target cells expressing CD19 and/or CD20 antigen. CEM (CD19−/CD20−) cells were used as a negative control. As shown in FIGS. 21A-D, all anti-CD19/CD20 CAR constructs exhibited cytotoxicity against Raji cells (WT and KO) but not cytotoxicity against CEM cells. Additionally, As shown in FIG. 22, IFN-γ secreted at 5:1 (E:T) showed increased cytokine production by all anti-CD19/CD-20 CAR T cells when co-cultured with either WT Raji cells, CD19 KO Raji cells, or CD20 KO Raji cells, with slightly higher IFN-γ secreted by CC314 cells (IgG4 hinge). As expected, CAR T cells co-cultured with CEM cells did not secrete IFN-γ.

### Example 12. Comparison of IMPT-514 with PBMC Manufactured with YESCARTA Construct or Breyanzi Construct

### T Cell Expansion

PBMCs from healthy and lupus nephritis (LN) donors were transduced with CC352 (anti-CD19 CAR) and expanded in media containing 300 IU/mL of IL-2 and 2.5% ICSR for 9 days. CD62L enriched cells were transduced with CC314B and expanded in media containing 50 IU/mL IL-2 and 0.5 ng/mL of IL-15 for 12 days to manufacture IMPT-514. IMPT-514 manufactured using CD62L+ cells isolated from LN donors showed reduced expansion compared to IMPT-514 manufactured using cells isolated from healthy donors (Table 13). This decrease in T cell expansion is due to reduced proliferation of T and B lymphocytes as a result of treatment with immunosuppressive regimens to control disease progression.

TABLE 13
Cell Expansion, CAR Expression, and
VCN of CD19 CAR T and IMPT-514

| | | | Fold | | VCN of |
|---|---|---|---|---|---|
| Donor | | | Expansion | | transduced |
| Status | Donor # | Product | from Day 3 | % CAR+ | cells |
| LN | PM04 | CD19 CAR T | 17 | 48 | 2.3 |
| | | IMPT-514 | 12 | 39 | 2.3 |
| | SB07 | CD19 CAR T | 23 | 39 | 1.8 |
| | | IMPT-514 | 25 | 37 | 1.8 |
| HD | D136 | CD19 CAR T | 13 | 39 | 1.7 |
| | | IMPT-514 | 38 | 51 | 2.4 |
| | D292 | CD19 CAR T | 15 | 36 | 1.8 |
| | | IMPT-514 | 65 | 46 | 2.7 |

CAR: chimeric antigen receptor; UTD: un-transduced; VCN: vector copy number. PBMCs isolated from 2 healthy donors and 2 LN donors were transduced with CC352 and harvested on day 9 (CD19 CAR T). CD62L enriched cells were transduced with CC314B and harvested on day 12 (IMPT-514). Genomic DNA was extracted from day 12 cell pellets from 2 healthy YESCART and IMPT-514 donors to assess VCN in the final product. Droplet digital polymerase chain reaction using specific primers/probe targeting Psi was performed to determine absolute VCN (A). VCN per transduced cells was calculated dividing the absolute VCN by the % of CAR positive cells (B). Figures depict experimental triplicates.

### Transgene Expression

Transduced PBMCs (anti-CD19 CAR T) or CD62L+ cells (IMPT-514) generated using starting material from healthy donors and LN donors were collected at harvest and stained using either anti-FMC 63 antibody to detect surface anti-CD19 CAR expression or anti-Leu16 scFv anti-idiotype to detect surface anti-CD19/CD20 CAR expression. Post-staining, cells were analyzed using Attune NxT flow cytometer to quantify percent CAR positive cells. Both LN and healthy donor CAR T cells demonstrated comparable CAR expression (36-51%) (Table 13).

CAR transgene integration was evaluated by measuring VCN. Genomic DNA was extracted from cell pellets of CD19 CAR T and IMPT-514 manufactured from two healthy donors. VCN per transduced cell was calculated by dividing total VCN by percentage of CAR-positive cells. VCN per transduced cell for CD19 CAR T donors were comparable to IMPT-514 (Table 13). In summary, VCNs for both products were fewer than 5 copies per transduced cell.

### T Cell Phenotype Characterization

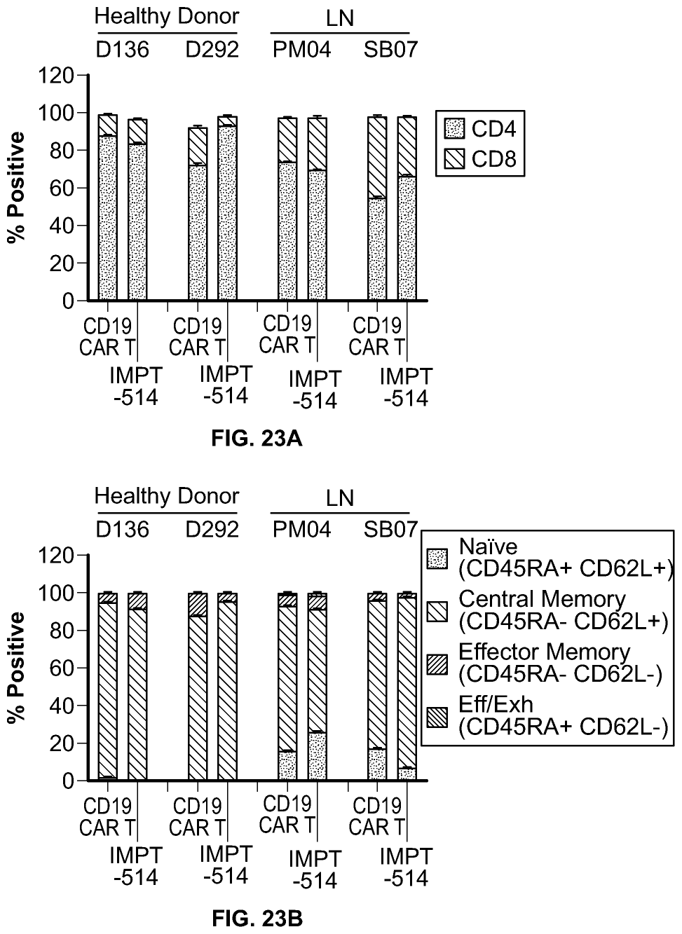

T cell phenotyping was performed on the day of harvest. Cells were pelleted and stained using appropriate antibodies. Post staining, cells were analyzed using Attune NxT flow cytometer (Thermo Fisher). SCC and isotype controls were also collected for each fluorophore. As shown in FIGS. 23A-B, both anti-CD19 CAR T cells and IMPT-514 manufactured from 2 healthy donors were composed of about 85%-90% central memory cells and 10%-15% effector memory cells. In addition, about 80%-90% of both anti-CD19 CAR T cells and IMPT-514 were CD4 T cells, with only about 10%-20% being CD8 T cells. Anti-CD19 CAR T cells and IMPT-514 manufactured from LN donors were composed of about 60-80% central memory cells, about 10-20% naïve/stem cell memory cells, and about 5% effector memory cells. LN donors had slightly higher % of CD8 cells (about 30-50%) compared to healthy donors.

### Potency Characterization in Raji B Cell Co-Culture Assay

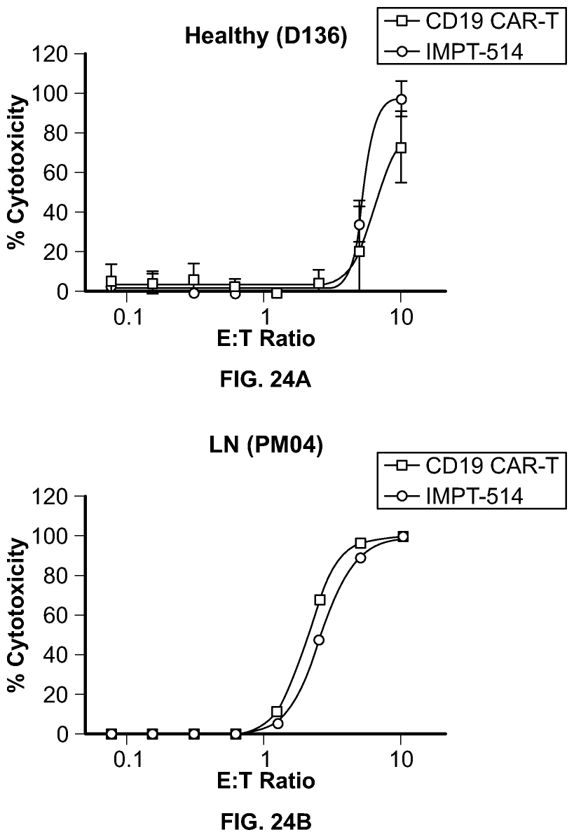

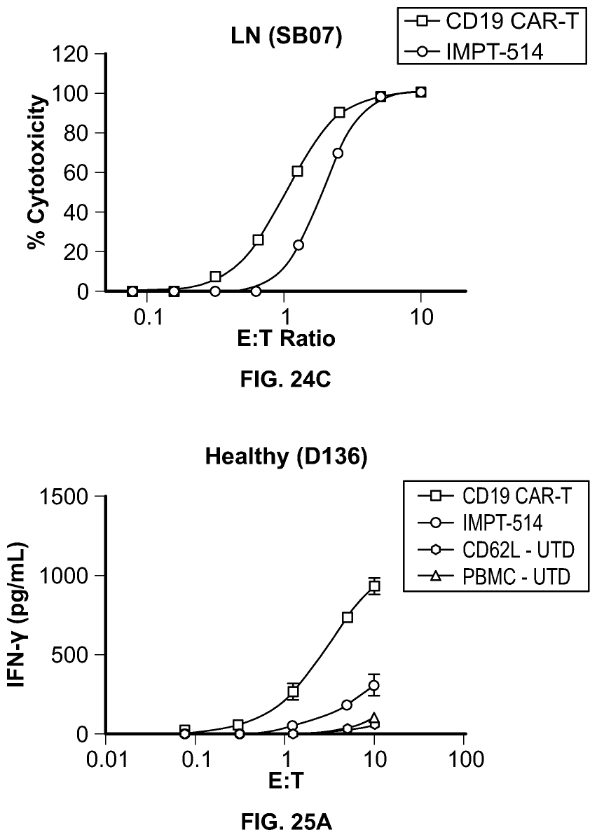

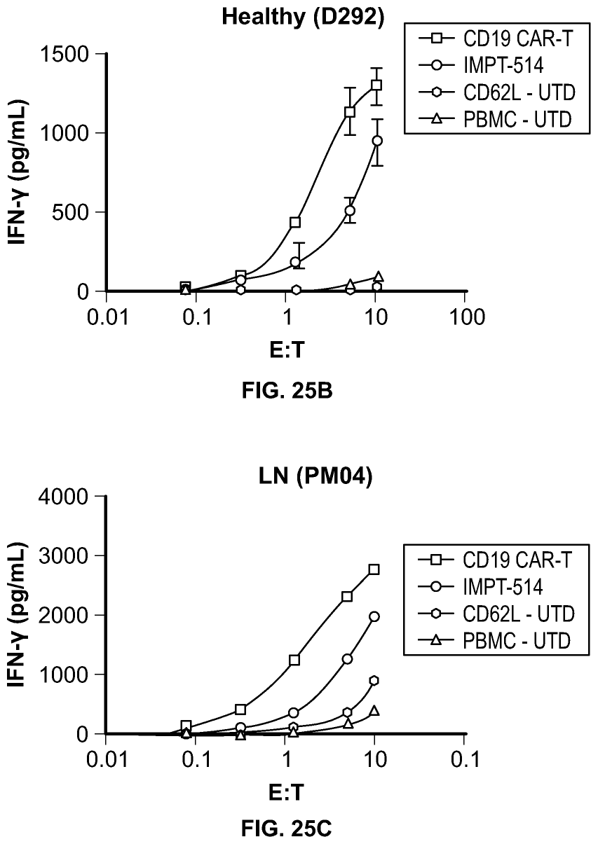

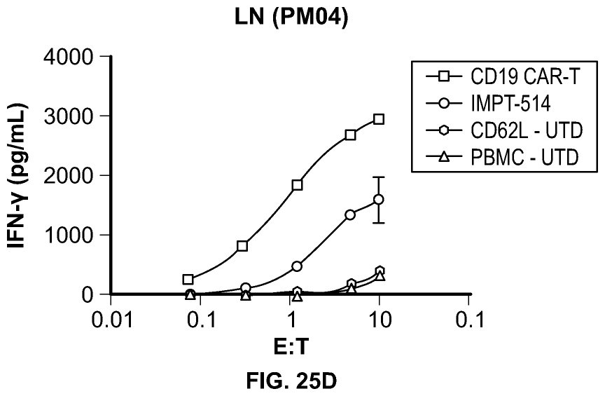

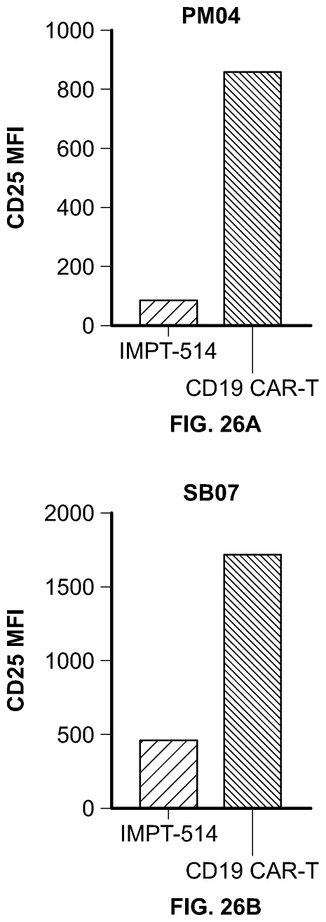

The potency of IMPT-514 and anti-CD19 CAR T cells were evaluated by co-culturing T cells with Raji B cells at various effector to target (E:T) ratios and measuring cytotoxicity and secreted cytokines. As shown in FIGS. 24A-C, cytotoxicity, measured by Raji cell viability 72 hours after co-culture, did not demonstrate significant differences between IMPT-514 and anti-CD19 CAR T cells across all donors. IFN-γ secreted in culture was quantified using Th1/Th2/Th17 cytometric bead array (CBA) kit (BD biosciences). As shown in FIGS. 25A-D, IMPT-514 showed reduced IFN-γ production compared with anti-CD19 CAR T cells for all LN and healthy donors, suggesting that IMPT-514 product potentially has benefit of reduced CRS compared to commercially available CD19 CAR T products. The lower IFN-γ production by IMPT-514 could be attributed to: (1) reduced inflammatory cytokine secreted by a more naïve and central memory (CM) selected CD62L population (Zah et al., Cancer Immunol. Res. 4(6): 498-508 (2016)); (2) 4-1BB co-stimulatory domain was associated with lower incidences of CRS and neurotoxicity compared to CD28 co-stimulatory domain in patients with r/r B-NHL (Ying et al., Mol. Ther. Oncolytics. 15:60-68 (2019)). For LN donors, IL-2 was lower for anti-CD19 CAR T cells (data not shown), likely due to increased consumption by actively proliferation CAR T cells in culture. To confirm this, both IMPT-514 and anti-CD19 CAR-T cells were stained to evaluate surface expression of CD25/IL-2Rα, a high affinity IL-2 receptor. As shown in FIGS. 26A-B, significantly higher levels of CD25 were observed on CD19 CAR-T cells compared to IMPT-514 for both LN donors. These results suggests that anti-CD19 CAR T cells, which are manufactured using a 9-day process, are more activated, with higher expression of CD25 and potentially increased consumption of IL-2, compared to IMPT-514 manufactured using a 12-day process.

### Comparison of PBMC Versus CD62L Starting Material for Engineering CAR T Cells

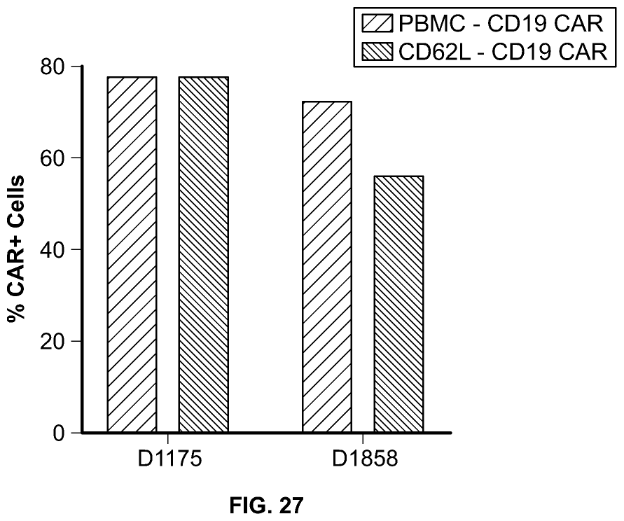

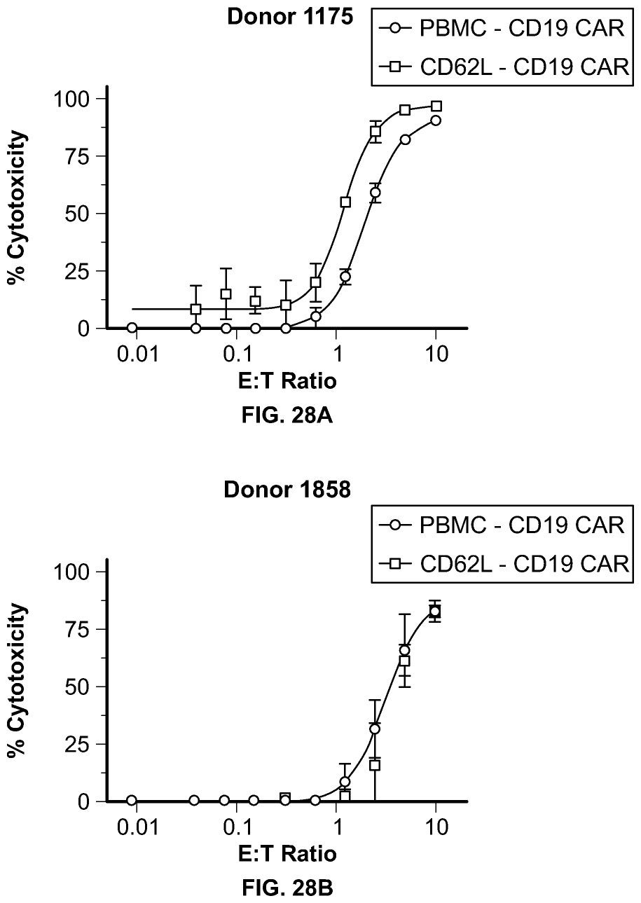

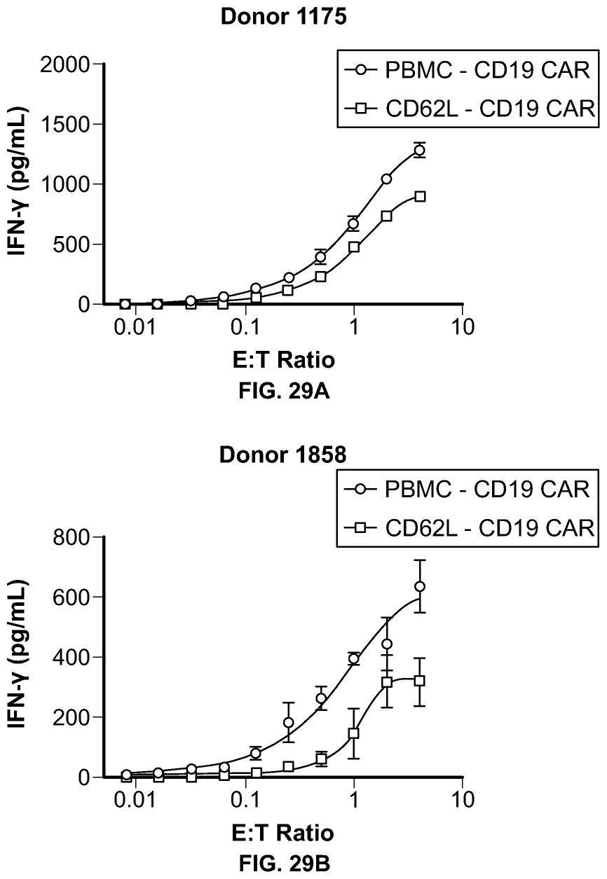

To further evaluate the contribution of the starting T cell material to CAR T cell cytokine secretion profile in response to antigen, PBMCs and CD62L enriched cells were transduced to express an anti-CD19 CAR (Breyanzi anti-CD19 CAR construct) following a manufacturing protocol similar to the one described above. As shown in FIG. 27, CAR expression was comparable for anti-CD19 CAR T cells manufactured from PBMC or CD62L cells. To compare potency of both CAR T products, CAR T cells manufactured from PBMCs (PBMC-CD19 CAR) and CAR T cells manufactured from CD62L+ cells (CD62L-CD19 CAR) were co-cultured with Raji B cells at various effector-to-target ratios. Raji B cell cytotoxicity was measured 72 hours after co-culture and pro-inflammatory cytokine secretion was measured 24 hours after co-culture. As shown in FIGS. 28A-B, PBMC-CAR and CD62L-CAR cells showed comparable killing of Raji B cells. However, IFN-γ production was greater in PBMC-CAR product compared to CD62L-CAR product, as shown in FIGS. 29A-B. IL-2, on the other hand, was slightly elevated in CD62L-CAR compared to PBMC-CAR (2 out of 3 donors) and TNF-α did not demonstrate a consistent trend among the 3 donors (data not shown).

### CONCLUSION/DISCUSSION

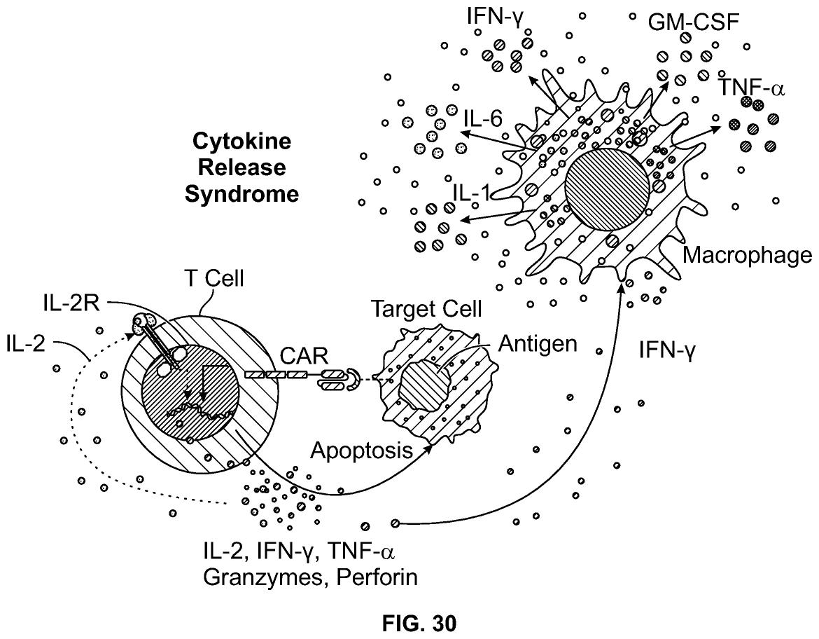

In this example, IMPT-514 was compared with anti-CD19 CAR T cells across 2 healthy donors and 2 LN donors. It was found that IMPT-514 demonstrated attenuated secretion of IFN-γ. IFN-γ was known to be a potent proinflammatory cytokine produced by Th1 cells and associated with development of cytokine release syndrome (CRS) in patients treated with CAR T therapy (FIG. 30; see also Teachey et al., Cancer Discov. 6(6):664-79 (2016)). Secreted IL-2 was variable across healthy and LN donors and was likely consumed by CAR T cells in culture to drive cell proliferation and as a result, concentrations varied from donor to donor. Similarly, variation in TNF-α secretion was observed across all donors (data not shown). This donor-to-donor variation of secreted TNF-α could be attributed to consumption by both T cells and Raji cells in culture. In summary, the reduction in secreted IFN-γ could be attributed to the naïve/memory CD62L+ starting cell population for IMPT-514 and the presence of 4-1BB costimulatory domain, both of which have been demonstrated in the literature to correspond to reduced toxicity and CRS in patients. In addition, a comparison of PBMC versus CD62L+ cells as starting materials for the manufacturing of CD19 CAR was performed. It was observed that IFN-γ secretion in cells manufactured with CD62L+ cells was reduced. Together, these results suggest that IMPT-514, which is manufactured using CD62L+ cells and contains a 4-1BB co-stimulatory domain, should be an effective and safe treatment with reduced risk of CRS for patients with lupus nephritis.

Since the IMPT-514 process begins with CD62L enrichment of naïve and memory T cells as the starting material, this helps limit the release of proinflammatory cytokines from the product in in vitro assays. Moreover, Arcangeli et. al., J. Clin. Invest. 132(12):e150807 (2022) recently shows that CAR T cells derived from these naïve/memory cells actively shape monocyte activation and appear more proficient at fine-tuning monocyte-derived cytokine release, with resultant reduction in IL-6 secretion. Thus, enrichment of naïve and memory T cells resulted in increased potency and expansion but was associated with limited CRS and neurotoxicity. It should also be noted that the IMPT-514 cryopreserved product is consequently enriched in central memory T cells relative to products made from PBMCs or bulk CD3+ cells. The central memory phenotype can also provide the ideal combination of reduced proinflammatory cytokine release, strong effector function, and long-term persistence of the CAR-T cell population.

### Example 13. Modified Procedure (8-Day) for Manufacturing IMPT-314 (and IMPT-514)

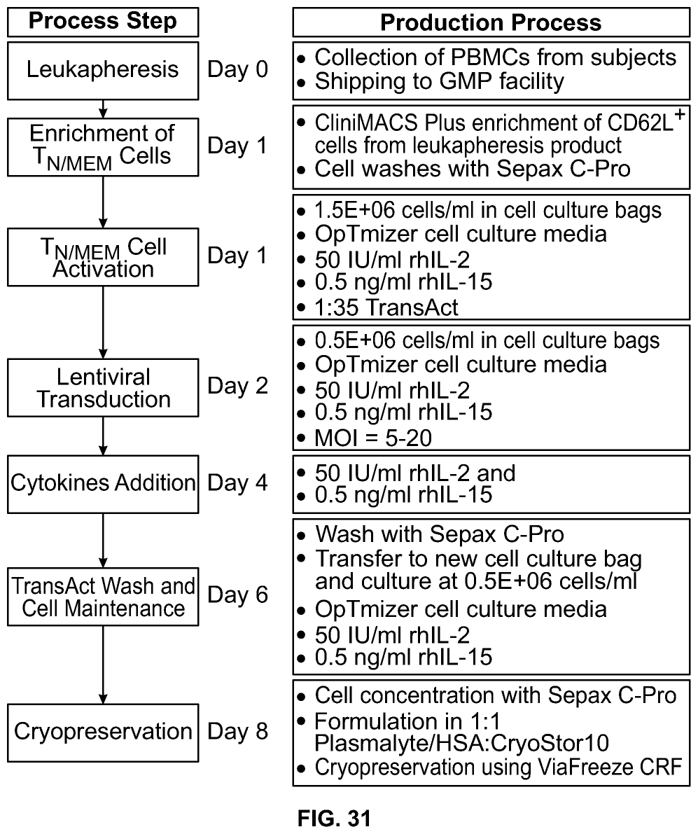

A modified manufacturing process for production of IMPT-314 is provided in FIG. 31. The manufacturing process consists of the following steps and timing:

### Day 0: Starting Material (Leukapheresis)

Autologous leukapheresis material is collected according to written procedures at pre-qualified apheresis centers and shipped using a qualified shipping container (at 2-8 degree temperature).

### Day 1: Enrichment and Activation of CD62L⁺ Cells

The leukapheresis product is enriched for CD62L+ cells using GMP-grade CD62L microbeads and CliniMACS Plus (Miltenyi Biotec) system. Briefly, the leukapheresis product is washed with CliniMACS buffer (phosphate buffered saline (PBS)/ethylenediaminetetraacetic acid (EDTA) buffer supplemented with 0.5% HSA) using a Sepax C-Pro (Cytiva). The cells are incubated with the anti-CD62L microbeads and washed with the CliniMACS buffer. CD62L labeled cells are then enriched using CliniMACS plus system following manufacturer's instructions. The resultant CD62L+ cells are resuspended in OpTmizer media supplemented with 50 IU/mL human IL-2 and 0.5 ng/mL human IL-15 (Miltenyi Biotec) and transferred to static cell culture bag (VueLife). The cells are stimulated with GMP-grade TransAct Human T cell expander CD3/CD28 (Miltenyi Biotec) at 1:35 ratio and cultured for about 24 hours at 37° C./5% carbon dioxide (CO2) in a humidified incubator.

### Day 2: Lentiviral Transduction

Activated CD62L⁺ cells are harvested from the static cell culture bag and re-seeded at 0.5×10⁶ cells/mL in a new cell culture bag. If recovered cell density is lower than 0.5×10⁶, a concentration step using Sepax C-Pro (Cytiva) will be performed. Lentiviral vector (CC314B) is added cells at a multiplicity of infection (MOI) of 5-15. The cells with the vector are incubated in a CO₂ incubator at 37±2° C. with 5±1% CO₂ and 95% RH (acceptable range 75% to 99%) for 2 days.

### Day 4: Media Feed

On Day 4 post-transduction, fresh media supplemented with cytokines (50 IU/mL human IL-2 and 0.5 ng/mL human IL-15 (Miltenyi Biotec) is added to the culture bag and incubated at 37±2° C. with 5±1% CO₂ and 95% RH (acceptable range 75% to 99%).

### Day 6: TransAct Wash and Cell Maintenance

On Day 6, transduced T cells are washed to remove residual TransAct using a Sepax C-Pro (Cytiva) and seeded in a fresh culture bag with media supplemented with cytokines at a density of 0.5×10⁶ cells/mL and incubated at 37±2° C. with 5±1% CO₂ and 95% RH (acceptable range 75% to 99%) for an additional 2 days.

### Day 8: Harvest and Cryopreservation

On Day 8, cells are harvested and washed to remove media using Plasmalyte plus HSA on Sepax C-Pro (Cytiva). If the target cell number for infusion is not reached at Day 8, cells will be kept in culture for 2 additional days (Day 10) or 4 additional days (Day 12) with regular media feeds with fresh cytokines.

The washed cells are formulated with CryoStor CS10 Freezing Solution (BioLife), filled into bags/vials at predetermined cell density and volume, and cryopreserved using a ViaFreeze controlled rate freezer (Cytiva). The final formulation is 50% Plasmalyte with 5% HSA and 50% CS10 (5% dimethylsulfoxide (DMSO)). Cryopreserved products are stored in vapor phase liquid nitrogen freezer.

### Product Comparison Between the 12-Day Process and the 8-Day Process

A total of seven donor leukapheresis lots were procured from commercial sources and 8-day manufacturing process side by side with 12-day manufacturing process from the same donor was performed. Across all seven donors, the incoming apheresis samples had about 7.8×10⁹±2.0×10⁹ total viable cells. Post-CD62L enrichment, the cell recovery was about 3.6×10⁹±0.83×10⁹ (about 47% recovery rate).

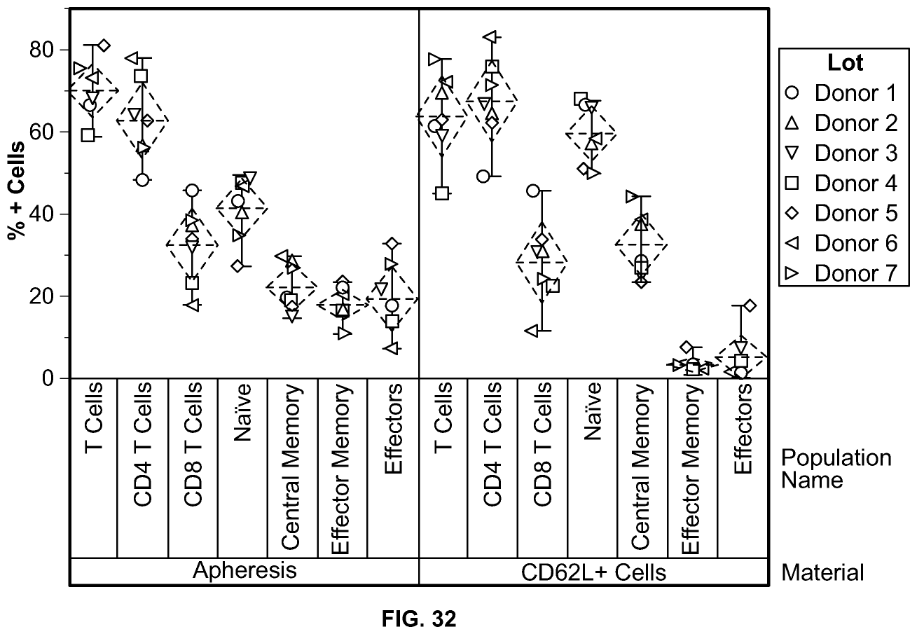

First, cell subset distribution was evaluated. As shown in FIG. 32, the cell subset distribution between apheresis and post-CD62L enrichment was similar, averaging about 65% T-cells and about 35% non-T-cells. Of note, the apheresis sample contains predominantly about 40% naïve T cells and 20% central memory cells; while CD62L enriched cells contain about 60% naïve cells and about 30% central memory T cells.

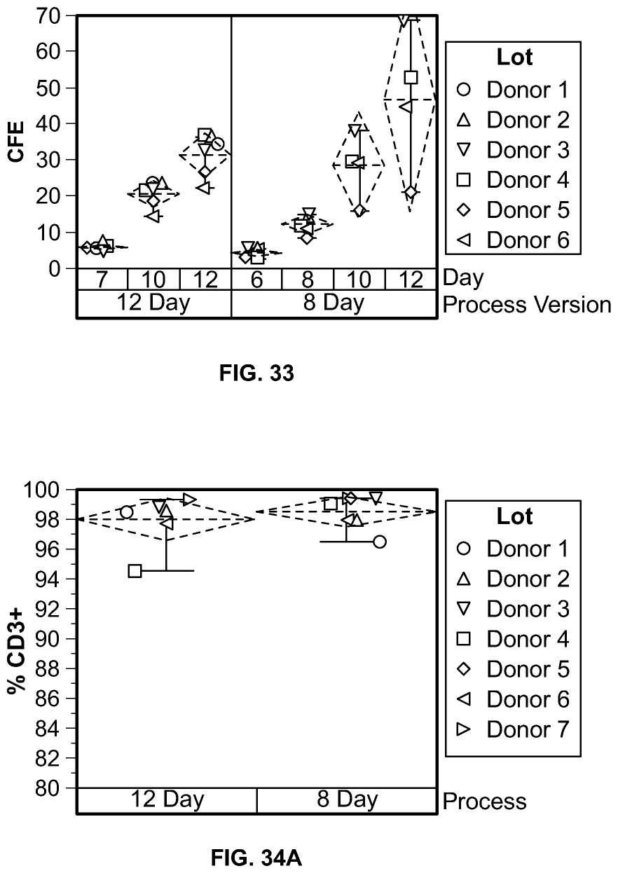

Second, cell expansion was evaluated. As shown in FIG. 33, the 12-day process resulted in an average cumulative fold expansion (CFE) about 30 fold at day 12; while the 8-day manufacturing process resulted in a CFE about 10 fold at day 8 and about 45 fold by day 12.

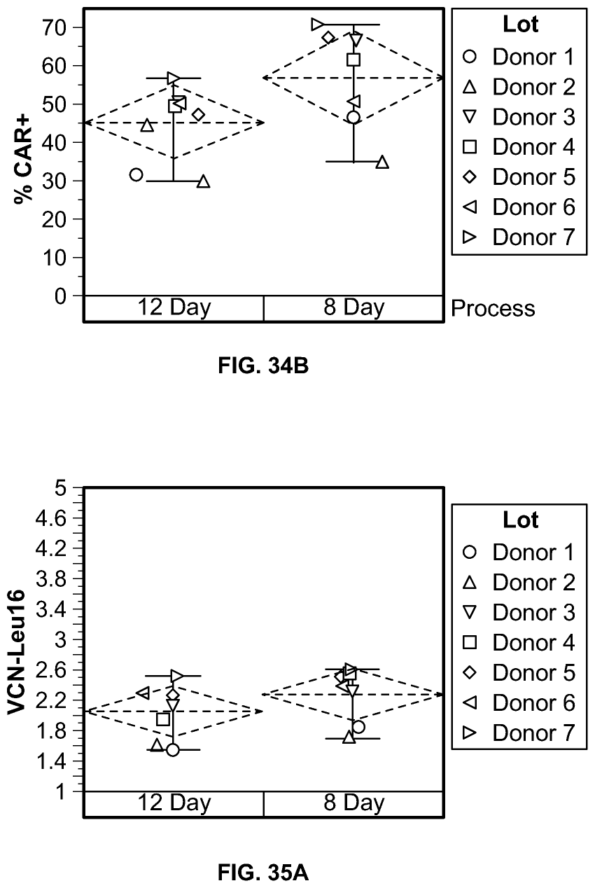

Third, CD3 and CAR expression was characterized and compared. Cells upon thaw from both manufacturing processes recovered well exhibiting greater than 85% viability. FIGS. 34A-B shows both CD3 and CAR expression comparison between the 8-day and 12-day manufacturing processes. Both manufacturing processes yielded greater than 90% CD3 composition of which 45-55% cells are CAR+. A slightly higher CAR expression was observed in the 8-day process.

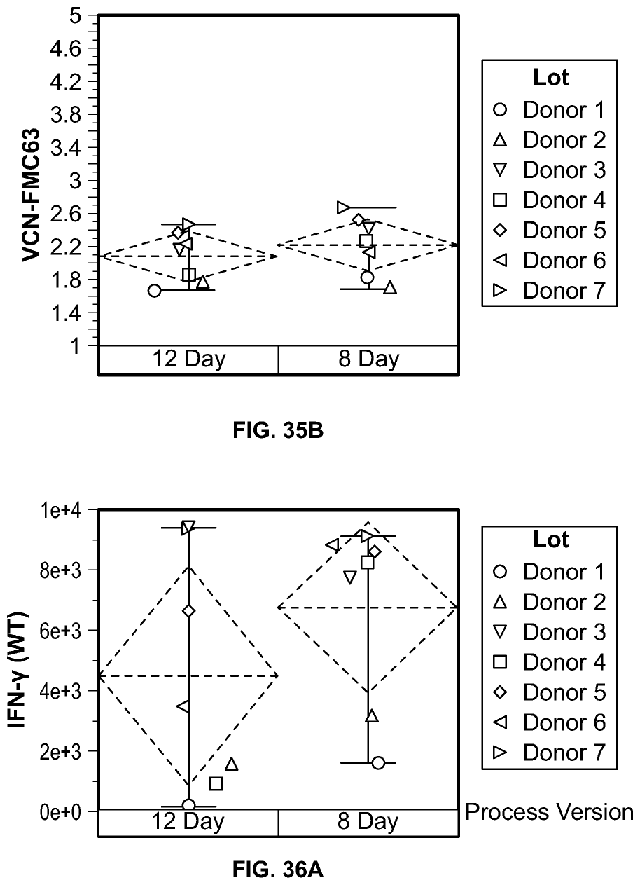

Fourth, vector copy number (VCN) was compared. As shown in FIGS. 35A-B, the 8-day process and the 12-day process showed comparable vector copy number (VCN) by detecting Leu16 or FMC63, both processes showing few than 5 copies per transduced cell.

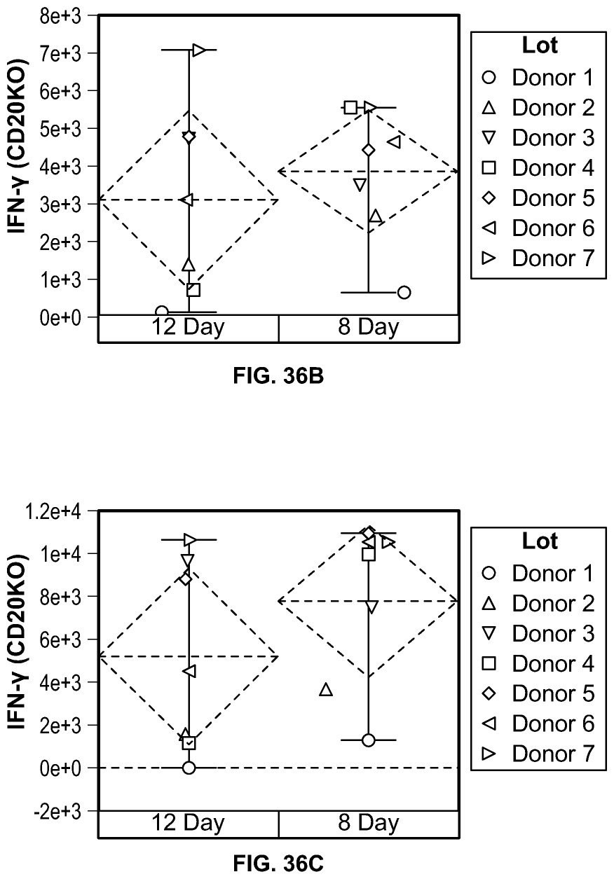

Fifth, IFN-γ secretion was compared between cells manufactured by the 12-day process and the 8-day process. As shown in FIGS. 36A-C, comparable levels of IFN-γ secretion was observed from the products manufactured using both processes against wild type, CD20KO, and CD19KO cell lines as targets.

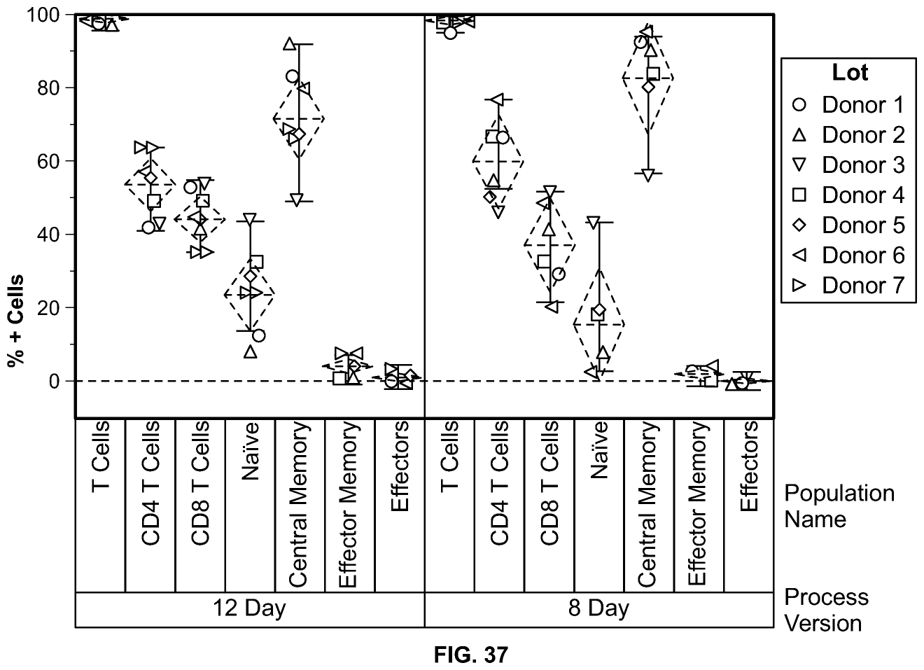

Sixth, T cell subsets were phenotypically characterized in the final drug products from both manufacturing processes. As shown in FIG. 37, comparable cell subset composition was observed in both. Each drug product contained predominantly a CD3+ product with about 70% or greater central memory subset and about 20% T-naïve cells.

### EXEMPLARY EMBODIMENTS

Embodiment 1. A closed system method of manufacturing a composition comprising a population of T cells, said method comprising the steps of:
a) isolating CD62L+ cells from a starting population of cells, thereby obtaining a population of naïve/memory T (T_{N/MEM}) cells, wherein a depletion of CD14+/CD25+ cells is not performed;
b) contacting said population of T_{N/MEM} cells with a transactivating agent to obtain a population of activated T_{N/MEM} cells;
c) transducing said population of activated T_{N/MEM} cells with a viral construct to obtain a population of transduced cells, wherein transducing is performed in the absence of at least one transduction enhancer selected from the group consisting of: polybrene, protamine sulfate, LentiBoost™, Vectofusin-1, and poloxamer;
d) expanding said population of transduced cells; and
e) removing the transactivating agent.

Embodiment 2. The method of embodiment 1, further comprising the following steps after step e):
f) expanding said population of transduced cells in the absence of said transactivating agent to obtain the composition comprising the population of T cells;
g) maintaining the composition comprising the population of T cells; and optionally
h) cryopreserving the composition comprising the population of T cells.

Embodiment 3. The method of embodiment 1 or 2, the starting population of cells of step a) is obtained from peripheral blood mononuclear cells (PBMCs) collected from an individual by leukapheresis.

Embodiment 4. The method of any one of embodiments 1 to 3, wherein isolating CD62L+ cells of step a) is achieved with CliniMACS® and CD62L microbeads.

Embodiment 5. The method of any one of embodiments 1 to 4, wherein said population of T_{N/MEM} cells is contacted with the transactivating agent for 18 to 48 hours, optionally about 18 hours, about 24 hours, about 30 hours, about 36 hours, about 42 hours, or about 48 hours, during step b).

Embodiment 6. The method of any one of embodiments 1 to 5, wherein MACS® GMP T cell TransAct™ is used as the transactivating agent.

Embodiment 7. The method of any one of embodiments 1 to 6, wherein said population of T_{N/MEM} cells is contacted with the transactivating agent in the presence of IL-2, IL-15, or both IL-2 and IL-15.

Embodiment 8. The method of any one of embodiments 1 to 7, wherein the viral construct of step c) is a lentiviral construct comprising a nucleic acid encoding a chimeric antigen receptor (CAR).

Embodiment 9. The method of embodiment 8, wherein the CAR is an anti-CD19/CD20 CAR comprising, in order:
an anti-CD20 scFv comprising (i) a light chain variable region having the amino acid sequence of SEQ ID NO: 34, and (ii) a heavy chain variable region having the amino acid sequences of SEQ ID NO: 35;
a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
an anti-CD19 scFv comprising (i) a heavy chain variable region having the amino acid sequence of SEQ ID NO: 36, and (ii) a light chain variable region having the amino acid sequences of SEQ ID NO: 37,
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

Embodiment 10. The method of embodiment 8 or 9, wherein the CAR comprises the amino acid sequence of any one selected from SEQ ID NOs: 2, 4, 6, 8, 10, 12, 14, 16, 18, or 20.

Embodiment 11. The method of any one of embodiments 8 to 10, wherein the CAR comprises the amino acid sequence of SEQ ID NO: 2.

Embodiment 12. The method of any one of embodiments 1 to 11, wherein transducing is performed at a multiplicity of infection (MOI) in the range of 5-20, optionally, about 5, about 10, about 15, or about 20, during step c).

Embodiment 13. The method of any one of embodiments 1 to 12, wherein transducing is performed in the absence of protamine sulfate during step c).

Embodiment 14. The method of any one of embodiments 1 to 13, wherein transducing is performed in the absence of a transduction enhancer during step c).

Embodiment 15. The method of any one of embodiments 1 to 14, wherein expanding is performed in the presence of IL-2 and IL-15 for about 24-120 hours, optionally, about 24 hours, about 36 hours, about 48 hours, about 60 hours, about 72 hours, about 84 hours, about 96 hours, about 108 hours, or about 120 hours during step d), and optionally wherein additional feed of IL-2 and IL-15 is supplemented.

Embodiment 16. The method of any one of embodiments 1 to 15, wherein step e) is performed by washing with Sepax C-Pro to remove the transactivating agent.

Embodiment 17. The method of any one of embodiments 1 to 16, wherein said population of transduced cells show improved quiescence after step e).

Embodiment 18. The method of any one of embodiments 2 to 17, wherein step g) is performed by placing the composition comprising the population of T cells in CTS™ OpTmizer™ media supplemented with IL-2 and IL-15 for about 48-144 hours, optionally, about 48 hours, about 60 hours, about 72 hours, about 84 hours, about 96 hours, about 108 hours, about 120 hours, about 132 hours, or about 144 hours.

Embodiment 19. The method of any one of embodiments 2 to 18, wherein cryopreserving is performed in CryoStor® CS10 media during step h).

Embodiment 20. A population of T cells manufactured by the method of any one of embodiments 1 to 19.

Embodiment 21. An anti-CD19/CD20 chimeric antigen receptor (CAR) comprising, in order:
an anti-CD20 scFv comprising (i) a light chain variable region having the amino acid sequence of SEQ ID NO: 34, and (ii) a heavy chain variable region having the amino acid sequences of SEQ ID NO: 35;
a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
an anti-CD19 scFv comprising (i) a heavy chain variable region having the amino acid sequence of SEQ ID NO: 36, and (ii) a light chain variable region having the amino acid sequences of SEQ ID NO: 37,
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

Embodiment 22. The CAR of embodiment 21, comprising the amino acid sequence of any one selected from SEQ ID NOs: 2, 4, 6, 8, 10, 12, 14, 16, 18, or 20.

Embodiment 23. The CAR of embodiment 21, comprising the amino acid sequence of SEQ ID NO: 2.

Embodiment 24. A nucleic acid encoding the CAR of any one of embodiments 21 to 23.

Embodiment 25. A plasmid comprising the nucleic acid of embodiment 24.

Embodiment 26. A vector comprising the nucleic acid of embodiment 24.

Embodiment 27. A cell comprising the nucleic acid of embodiment 24.

Embodiment 28. The cell of embodiment 27, wherein the cell is a T cell.

Embodiment 29. A method of treating a disease or disorder comprising administering to a patient an effective amount of the population of T cells of embodiment 20.

Embodiment 30. The method of embodiment 29, wherein said disease or disorder is at least one of lymphoma, leukemia, glioma, and/or glioblastoma.

Embodiment 31. The method of embodiment 30, wherein said lymphoma is NHL, DLBCL, follicular lymphoma, MALT, CTCL, or MCL.

What is claimed is:

1. A closed system method of manufacturing a composition comprising a population of T cells, said method comprising the steps of:
a) isolating CD62L+ cells from a starting population of cells, thereby obtaining a population of naïve/memory T (T_{N/MEM}) cells, wherein a depletion of CD14+/CD25+ cells is not performed;
b) contacting said population of T_{N/MEM} cells with a transactivating agent to obtain a population of activated T_{N/MEM} cells;
c) transducing said population of activated T_{N/MEM} cells with a viral construct to obtain a population of transduced cells, wherein transducing is performed in the absence of at least one transduction enhancer selected from the group consisting of: polybrene, protamine sulfate, LentiBoost™, Vectofusin-1, and poloxamer;
d) expanding said population of transduced cells; and
e) removing the transactivating agent.

2. The method of claim 1, further comprising the following steps after step e):
f) expanding said population of transduced cells in the absence of said transactivating agent to obtain the composition comprising the population of T cells;
g) maintaining the composition comprising the population of T cells; and optionally
h) cryopreserving the composition comprising the population of T cells.

3. The method of claim 1, the starting population of cells of step a) is obtained from peripheral blood mononuclear cells (PBMCs) collected from an individual by leukapheresis.

4. The method of claim 1, wherein isolating CD62L+ cells of step a) is achieved with CliniMACS® and CD62L microbeads.

5. The method of claim 1, wherein said population of T_{N/MEM} cells is contacted with the transactivating agent for 18 to 48 hours during step b).

6. The method of claim 5, wherein MACS® GMP T cell TransAct™ is used as the transactivating agent.

7. The method of claim 5, wherein said population of T_{N/MEM} cells is contacted with the transactivating agent in the presence of IL-2, IL-15, or both IL-2 and IL-15.

8. The method of claim 1, wherein the viral construct of step c) is a lentiviral construct comprising a nucleic acid encoding a chimeric antigen receptor (CAR).

9. The method of claim 8, wherein the CAR is an anti-CD19/CD20 CAR comprising, in order:
an anti-CD20 scFv comprising (i) a light chain variable region having the amino acid sequence of SEQ ID NO: 34, and (ii) a heavy chain variable region having the amino acid sequences of SEQ ID NO: 35;
a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
an anti-CD19 scFv comprising (i) a heavy chain variable region having the amino acid sequence of SEQ ID NO: 36, and (ii) a light chain variable region having the amino acid sequences of SEQ ID NO: 37,
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

10. The method of claim 9, wherein the CAR comprises the amino acid sequence of any one selected from SEQ ID NOs: 2, 4, 6, 8, 10, 12, 14, 16, 18, or 20.

11. The method of claim 1, wherein transducing is performed at a multiplicity of infection (MOI) in the range of 5-20 during step c).

12. The method of claim 1, wherein transducing is performed in the absence of protamine sulfate during step c).

13. The method of claim 12, wherein transducing is performed in the absence of a transduction enhancer during step c).

14. The method of claim 1, wherein expanding is performed in the presence of IL-2 and IL-15 for about 24-120 hours during step d), and wherein additional feed of IL-2 and IL-15 is supplemented.

15. The method of claim 1, wherein step e) is performed by washing with Sepax C-Pro to remove the transactivating agent.

16. The method of claim 1, wherein said population of transduced cells show improved quiescence after step e).

17. The method of claim 2, wherein step g) is performed by placing the composition comprising the population of T cells in CTS™ OpTmizer™ media supplemented with IL-2 and IL-15 for about 48-144 hours.

18. The method of claim 2, wherein cryopreserving is performed in CryoStor® CS10 media during step h).

19. A population of T cells manufactured by the method of any one of claims 1 to 18.

20. An anti-CD19/CD20 chimeric antigen receptor (CAR) comprising, in order:
an anti-CD20 scFv comprising (i) a light chain variable region having the amino acid sequence of SEQ ID NO: 34, and (ii) a heavy chain variable region having the amino acid sequences of SEQ ID NO: 35;
a glycine-serine (GS) flexible linker selected from the group consisting of G₄S (SEQ ID NO: 48), (G₄S)₂ (SEQ ID NO: 49), (G₄S)₃ (SEQ ID NO: 50), and (G₄S)₄ (SEQ ID NO: 47);
an anti-CD19 scFv comprising (i) a heavy chain variable region having the amino acid sequence of SEQ ID NO: 36, and (ii) a light chain variable region having the amino acid sequences of SEQ ID NO: 37,
a spacer domain comprising the amino acid sequence of any one selected from SEQ ID NOs: 21-29;
a transmembrane domain comprising the amino acid sequence of SEQ ID NO: 30 or 31;
a 4-1BB cytoplasmic signaling domain comprising the amino acid sequence of SEQ ID NO: 32; and
a CD3 zeta signaling domain comprising the amino acid sequence of SEQ ID NO: 33.

21. The CAR of claim 20, comprising the amino acid sequence of any one selected from SEQ ID NOs: 2, 4, 6, 8, 10, 12, 14, 16, 18, or 20.

22. A nucleic acid encoding the CAR of claim 20.

23. A T cell comprising the nucleic acid of claim 22.


SEQUENCE LISTING (50 sequences)

SEQ ID NO: 1 - DNA, 2178 bp, synthetic construct
  source 1..2178: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 2 - protein, 725 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 2277 bp, synthetic construct
  source 1..2277: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccactacaactcctgccccacggccacctacacctgcgcctacaattgccagccagccactgtccttacgaccagaggcttgccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 4 - protein, 758 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2259 bp, synthetic construct
  source 1..2259: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccattgaagttatgtatcctcctccttacctagacaatgagaagagcaatggaaccattatccatgtgaaagggaaacacctttgtccaagtcccctatttcccggaccttctaagcccatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtga

SEQ ID NO: 6 - protein, 752 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIEVMYPPPYLDNEKSNGTIIHVKGKHLCPSPLFPGPSKPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 2286 bp, synthetic construct
  source 1..2286: mol_type = other DNA
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccactacaactcctgccccacggccacctacacctgcgcctacaattgccagccagccactgtccttacgaccagaggcttgccgccccgctgcgggaggcgccgtccatacccggggccttgacttcgcatgcgatatttacatttgggcacccttggccggcacctgcggagttttacttctgagcttggtgataacgctgtactgtaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 8 - protein, 754 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 2214 bp, synthetic construct
  source 1..2214: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcacctctgggcaagttcttcttgagtctaatatcaaggtgctgcccacctggtctactcctgtccaaccgatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 10 - protein, 735 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGQVLLESNIKVLPTWSTPVQPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 2235 bp, synthetic construct
  source 1..2235: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccagccagccactgtccttacgaccagaggcttgccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 12 - protein, 742 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSQPLSLRPEACRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 2202 bp, synthetic construct
  source 1..2202: mol_type = other DNA
atggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcacccgccccgctgcgggaggcgccgtccatacccggggccttgacttcgcatgcgatatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 14 - protein, 731 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRPAAGGAVHTRGLDFACDMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 2199 bp, synthetic construct
  source 1..2199: mol_type = other DNA
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagcctaaaagttgtgataaaacgcatacctgtccaatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 16 - protein, 725 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITEPKSCDKTHTCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 2199 bp, synthetic construct
  source 1..2199: mol_type = other DNA
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgagccaaagtcatgtgatacacctcctccctgtcccatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 18 - protein, 725 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITEPKSCDTPPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 2205 bp, synthetic construct
  source 1..2205: mol_type = other DNA
gctagcgccgccaccatggagacagacacactcctgctatgggtgctgctgctctgggttccaggttccacaggtgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagcggtggaggcggcagtggcggaggtgggagcggagggggcggttccggtggcgggggatctgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccgtaccctgtcgagtaccgcctccgcccccatgctgccacccgatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgagtcgac

SEQ ID NO: 20 - protein, 727 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITVPCRVPPPPPCCHPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 12 aa, synthetic construct
ESKYGPPCPPCP

SEQ ID NO: 22 - protein, 45 aa, synthetic construct
TTTPAPRPPTPAPTIASQPLSLRPEACRPAAGGAVHTRGLDFACD

SEQ ID NO: 23 - protein, 39 aa, synthetic construct
IEVMYPPPYLDNEKSNGTIIHVKGKHLCPSPLFPGPSKP

SEQ ID NO: 24 - protein, 22 aa, synthetic construct
SGQVLLESNIKVLPTWSTPVQP

SEQ ID NO: 25 - protein, 29 aa, synthetic construct
SQPLSLRPEACRPAAGGAVHTRGLDFACD

SEQ ID NO: 26 - protein, 18 aa, synthetic construct
RPAAGGAVHTRGLDFACD

SEQ ID NO: 27 - protein, 12 aa, synthetic construct
EPKSCDKTHTCP

SEQ ID NO: 28 - protein, 12 aa, synthetic construct
EPKSCDTPPPCP

SEQ ID NO: 29 - protein, 14 aa, synthetic construct
VPCRVPPPPPCCHP

SEQ ID NO: 30 - protein, 27 aa, synthetic construct
FWVLVVVGGVLACYSLLVTVAFIIFWV

SEQ ID NO: 31 - protein, 24 aa, synthetic construct
IYIWAPLAGTCGVLLLSLVITLYC

SEQ ID NO: 32 - protein, 42 aa, synthetic construct
KRGRKKLLYIFKQPFMRPVQTTQEEDGCSCRFPEEEEGGCEL

SEQ ID NO: 33 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 35 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 36 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 38 - DNA, 22 bp, synthetic construct
  source 1..22: mol_type = other DNA
cttattactgccagcagtggag

SEQ ID NO: 39 - DNA, 20 bp, synthetic construct
  source 1..20: mol_type = other DNA
caggacatcttcactgaggc

SEQ ID NO: 40 - DNA, 23 bp, synthetic construct
  source 1..23: mol_type = other DNA
cggagccaccaccgctagtactg

SEQ ID NO: 41 - DNA, 19 bp, synthetic construct
  source 1..19: mol_type = other DNA
cgacatccagatgacccag

SEQ ID NO: 42 - DNA, 20 bp, synthetic construct
  source 1..20: mol_type = other DNA
gttccaggttggagatggtc

SEQ ID NO: 43 - DNA, 29 bp, synthetic construct
  source 1..29: mol_type = other DNA
caccgtcaagctgctgatctaccacacca

SEQ ID NO: 44 - DNA, 19 bp, synthetic construct
  source 1..19: mol_type = other DNA
cagggtctgggagccaagt

SEQ ID NO: 45 - DNA, 21 bp, synthetic construct
  source 1..21: mol_type = other DNA
gcacagtgctggacattgaca

SEQ ID NO: 46 - DNA, 30 bp, synthetic construct
  source 1..30: mol_type = other DNA
cccaccgaacccaagaaactagaggagaat

SEQ ID NO: 47 - protein, 20 aa, synthetic construct
GGGGSGGGGSGGGGSGGGGS

SEQ ID NO: 48 - protein, 5 aa, synthetic construct
GGGGS

SEQ ID NO: 49 - protein, 10 aa, synthetic construct
GGGGSGGGGS

SEQ ID NO: 50 - protein, 15 aa, synthetic construct
GGGGSGGGGSGGGGS